(19) United States
(12) Patent Application Publication
(10) Pub. No.: US 2004/0009506 A1
(43) Pub. Date: Jan. 15, 2004

(54) Methods and compositions for detection and quantitation of nucleic acid analytes

(75) Inventors: Jean-Philippe F. Stephan, San Carlos, CA (US); Siao Ping Tsai, South San Francisco, CA (US); Wai Lee Tan Wong, Los Altos, CA (US); Todd Billeci, Pittsburg, CA (US)

Correspondence Address: GENENTECH, INC., 1 DNA WAY, SOUTH SAN FRANCISCO, CA, 94080, US

(73) Assignee: GENENTECH, INC.

(21) Appl. No.: 10/401,520

(22) Filed: Mar. 28, 2003

Related U.S. Application Data
(60) Provisional application No. 60/368,669, filed on Mar. 29, 2002.

(51) Int. Cl. C12Q 1/68

(52) U.S. Cl. 435/6

(57) ABSTRACT

The present invention provides novel solution phase hybridization-based methods for detecting and quantitating nucleic acid analytes. Methods comprising use of novel capture polymers and/or signaling systems are provided. Use of these novel capture polymers and/or signaling systems provides significant improvements in signal to noise ratio, specificity, sensitivity and ease of development and use as compared to existing solution phase nucleic acid detection and quantitation methods. The invention further provides compositions, kits and articles of manufacture for practicing methods of the present invention.

### RELATED APPLICATIONS

This application claims priority benefit of U.S. provisional application Serial No. 60/368,669, filed Mar. 29, 2002, which is incorporated herein by reference in its entirety.

### TECHNICAL FIELD

The invention relates to the field of nucleic acid detection and quantitation. More particularly, the invention provides methods, compositions, kits and articles of manufacture for solution phase hybridization-based nucleic acid detection and quantitation.

### BACKGROUND

Since their initial development two decades ago, nucleic acid hybridization methods have been widely used in genetic, biomedical research and clinical laboratories for various applications such as the identification, structure analysis and determination of function of genes and their transcripts, such as those of viruses, bacteria and parasites. A variety of approaches have been developed, including direct blotting methods and solution phase hybridization (capture-based) methods.

In direct blotting methods, the nucleic acid analyte is directly applied to a solid support and subsequently hybridized with a labeled DNA fragment. These methods have generally been considered the method of choice in terms of sensitivity. Solution phase hybridization methods are generally based on capture of target nucleic acids using synthetic nucleic acid oligonucleotides that are immobilized on a solid support.

Blotting-based methods are not amenable to detecting and quantitating nucleic acid analytes suspected or known to be present in a complex mixture containing large numbers of non-target nucleic acid sequences. Solution phase capture-based assays are generally used for this purpose. However, the presence of large numbers of contaminants often significantly compromises specific signal due to partial hybridization of the contaminants with capture oligonucleotides. In cases where the sample is an ex vivo/vitro extract, which usually contains proteins and other biomolecules, signal specificity may be negatively affected via numerous undesirable macromolecular interactions.

One form of solution phase hybridization assay utilizes a capture oligonucleotide that is indirectly attached to a solid support through universal oligonucleotides. See, for e.g., Urdea et al., U.S. Pat. Nos. 5,635,352; 5,681,697. However, use of universal oligonucleotides limits the ability to adapt such assays to array formats wherein each array spot comprises more than one species of oligonucleotide. Generally, only one “universal” sequence can be provided in each array spot. This poses a formidable challenge in adapting the assay to an array format.

Existing solution phase hybridiztion methods require components that can be cumbersome to design, synthesize and/or use. Numerous attempts have been made to improve component oligonucleotides for use in hybridization-based assays. See, for e.g., Collins et al., U.S. Pat. Nos. 5,780,610; 5,681,702; 5,736,327; 5,747,248. Similarly, attempts at developing signal amplification systems for use in these assays have led to various configurations of amplification oligonucleotides, such as the “branched multimer” of Urdea et al., See, for e.g., U.S. Pat. Nos. 5,849,481; 5,624,802; 5,710,264 & 5,124,246. Branched multimers are highly complex polynucleotides that comprise a polynucleotide backbone having at least 15 multifunctional nucleotides, each of which defines a sidechain site and a single-stranded oligonucleotide unit that is capable of binding to a polynucleotide of interest.

The intrinsic problems of nucleic acid detection and quantitation, which conventional methods have not adequately overcome, continue to present significant obstacles towards development of assays that can provide adequate, sensitive and reliable signal/noise ratios, while retaining flexibility of design (for e.g., flexible design of assay components such as signal amplification oligonucleotides), versatility, and ease of use and development. Moreover, the increased number of genes that have been identified, and the increasing focus on genomics-based research and therapeutic approaches call for assay methods that can provide high throughput nucleic acid detection and quantitation, such as through the use of arrays/microaarays and automation of assay methods.

Therefore, there is a need for improved solution phase capture-based nucleic acid detection and quantitation methods that overcome drawbacks in existing methods. The invention provided herein fulfills this need and provides additional benefits.

All references cited herein, including patent applications and publications, are incorporated by reference in their entirety.

### DISCLOSURE OF THE INVENTION

The invention provides methods and compositions for detection and quantitation of nucleic acid analytes, as well as applications of the methods.

Accordingly, in one aspect, the invention provides a method for detecting or quantitating a nucleic acid analyte in a sample, said method comprising: (A) contacting said sample with an analyte-binding oligonucleotide, a labeled oligonucleotide, a capture polymer and a linear stem oligonucleotide under conditions whereby a complex is formed comprising the analyte, analyte-binding oligonucleotide, labeled oligonucleotide, capture polymer and linear stem oligonucleotide, wherein: (i) the analyte-binding oligonucleotide comprises (a) a sequence that is hybridizable to the analyte and (b) a sequence that is hybridizable to the stem oligonucleotide; (ii) the linear stem oligonucleotide comprises (a) a sequence that is hybridizable to the analyte-binding oligonucleotide and (b) a sequence that is directly or indirectly hybridizable to the labeled oligonucleotide; (iii) the labeled oligonucleotide comprises (a) a sequence that is directly or indirectly hybridizable to the stem oligonucleotide and (b) a label capable of directly or indirectly generating a detectable signal; (iv) the capture polymer comprises a nucleic acid sequence that is directly or indirectly hybridizable to the analyte; and (B) detecting or quantitating the complex of step (A); whereby detection or quantitation of the complex of step (A) is indicative of presence or quantity of the nucleic acid analyte in the sample. In one embodiment, the labeled oligonucleotide is a linear oligonucleotide. In some embodiments, the labeled oligonucleotide is a linear labeled oligonucleotide that comprises two or more units of label each attached directly to the oligonucleotide. In one embodiment, the linear stem oligonucleotide comprises a sequence that is directly hybridizable to the labeled oligonucleotide and the labeled oligonucleotide comprises a sequence that is directly hybridizable to the stem oligonucleotide.

In another aspect, the invention provides a method for detecting or quantitating a nucleic acid analyte in a sample, said method comprising: (A) contacting said sample with an analyte-binding oligonucleotide, a linear labeled oligonucleotide and a capture polymer under conditions whereby a complex is formed comprising the analyte, analyte-binding oligonucleotide, linear labeled oligonucleotide and capture polymer, wherein: (i) the analyte-binding oligonucleotide comprises (a) a sequence that is hybridizable to the analyte and (b) a sequence that is hybridizable to the linear labeled oligonucleotide; (ii) the linear labeled oligonucleotide comprises (a) at least two or more units of label each attached directly to the oligonucleotide and (b) a sequence that is hybridizable to the analyte-binding oligonucleotide; (iii) the capture polymer comprises a nucleic acid sequence that is directly or indirectly hybridizable to the analyte; and (B) detecting or quantitating the complex of step (A); whereby detection or quantitation of the complex of step (A) is indicative of presence or quantity of the nucleic acid analyte in the sample.

In yet another aspect, the invention provides a method for detecting or quantitating a nucleic acid analyte in a sample, said method comprising: (A) contacting said sample with an analyte-binding linear labeled oligonucleotide and a capture polymer under conditions whereby a complex is formed comprising the analyte, analyte-binding linear labeled oligonucleotide and capture polymer, wherein: (i) the analyte-binding linear labeled oligonucleotide comprises (a) a sequence that is hybridizable to the analyte and (b) two or more units of label each attached directly to the oligonucleotide; (ii) the capture polymer comprises a nucleic acid sequence that is directly or indirectly hybridizable to the analyte; and (B) detecting or quantitating the complex of step (A); whereby detection or quantitation of the complex of step (A) is indicative of presence or quantity of the nucleic acid analyte in the sample.

In another aspect, the invention provides a method for detecting or quantitating a nucleic acid analyte in a sample, said method comprising: (a) contacting the sample with an analyte-binding oligonucleotide and a capture polymer under conditions whereby a complex is formed comprising the analyte, analyte-binding oligonucleotide, and capture polymer, wherein: (i) the analyte-binding oligonucleotide comprises a sequence that is hybridizable to the analyte; and (ii) the capture polymer comprises a first portion that is hybridizable to the analyte and a second portion comprising a material (preferably, but not necessarily, a non-nucleic acid material) that is not substantially hybridizable to nucleic acid; and (b) detecting or quantitating the complex of step (a); whereby detection or quantitation of the complex of step (a) is indicative of presence or quantity of the nucleic acid analyte in the sample.

In another aspect, the invention provides a method for detecting or quantitating a nucleic acid analyte in a sample, said method comprising: (a) contacting the sample with an analyte-binding oligonucleotide and a capture polymer under conditions whereby a complex is formed comprising the analyte, analyte-binding oligonucleotide, and capture polymer, wherein: (i) the analyte-binding oligonucleotide comprises a sequence that is hybridizable to the analyte; and (ii) the capture polymer comprises a sequence that is hybridizable to the analyte and further comprises at least one modified nucleotide that enhances strength of hybridization of the polymer to the analyte; and (b) detecting or quantitating the complex of step (a); whereby detection or quantitation of the complex of step (a) is indicative of presence or quantity of the nucleic acid analyte in the sample.

In another aspect, the invention provides a method for detecting or quantitating a nucleic acid analyte in a sample, said method comprising: (a) contacting the sample with an analyte-binding oligonucleotide and a capture polymer under conditions whereby a complex is formed comprising the analyte, analyte-binding oligonucleotide, and capture polymer, wherein: (i) the analyte-binding oligonucleotide comprises a sequence that is hybridizable to the analyte; and (ii) the capture polymer comprises a first portion that is hybridizable to the analyte, said first portion comprising at least one modified nucleotide that enhances strength of hybridization of the polymer to the analyte, and a second portion comprising a material (preferably but not necessarily a non-nucleic acid material) that is not substantially hybridizable to nucleic acid; and (b) detecting or quantitating the complex of step (a); whereby detection or quantitation of the complex of step (a) is indicative of presence or quantity of the nucleic acid analyte in the sample.

In one aspect, the invention provides a method for detecting or quantitating a nucleic acid analyte in a sample, said method comprising: (A) contacting said sample with an analyte-binding oligonucleotide, a labeled oligonucleotide, a capture polymer and a linear stem oligonucleotide under conditions whereby a complex is formed comprising the analyte, analyte-binding oligonucleotide, labeled oligonucleotide, capture polymer and linear stem oligonucleotide, wherein: (i) the analyte-binding oligonucleotide comprises a sequence that is hybridizable to the analyte and a sequence that is hybridizable to the stem oligonucleotide; (ii) the stem oligonucleotide comprises (a) a sequence that is hybridizable to the analyte-binding oligonucleotide and (b) a sequence that is directly or indirectly hybridizable to the labeled oligonucleotide; (iii) the labeled oligonucleotide comprises (a) a sequence that is directly or indirectly hybridizable to the stem oligonucleotide and (b) a label capable of directly or indirectly generating a detectable signal; (iv) the capture polymer comprises a first portion that is hybridizable to the analyte and and a second portion comprising a material (preferably but not necessarily a non-nucleic acid material) that is not substantially hybridizable to nucleic acid; and (B) detecting or quantitating the complex of step (A); whereby detection or quantitation of the complex of step (A) is indicative of presence or quantity of the nucleic acid analyte in the sample. In one embodiment, the labeled oligonucleotide is a linear oligonucleotide. In some embodiments, the labeled oligonucleotide is a linear labeled oligonucleotide that comprises two or more units of label each attached directly to the oligonucleotide. In one embodiment, the linear stem oligonucleotide comprises a sequence that is directly hybridizable to the labeled oligonucleotide and the labeled oligonucleotide comprises a sequence that is directly hybridizable to the stem oligonucleotide.

In still another aspect, the invention provides a method for detecting or quantitating a nucleic acid analyte in a sample, said method comprising: (A) contacting said sample with an analyte-binding oligonucleotide, a linear labeled oligonucleotide and a capture polymer under conditions whereby a complex is formed comprising the analyte, analyte-binding oligonucleotide, linear labeled oligonucleotide and capture polymer, wherein: (i) the analyte-binding oligonucleotide comprises (a) a sequence that is hybridizable to the analyte and (b) a sequence that is hybridizable to the linear labeled oligonucleotide; (ii) the linear labeled oligonucleotide comprises (a) two or more units of label each attached directly to the oligonucleotide and (b) a sequence that is hybridizable to the analyte-binding oligonucleotide; (iii) the capture polymer comprises a first portion that is hybridizable to the analyte and and a second portion comprising a material (preferably but not necessarily a non-nucleic acid material) that is not substantially hybridizable to nucleic acid; and (B) detecting or quantitating the complex of step (A); whereby detection or quantitation of the complex of step (A) is indicative of presence or quantity of the nucleic acid analyte in the sample.

In one aspect, the invention provides a method for detecting or quantitating a nucleic acid analyte in a sample, said method comprising: (A) contacting said sample with an analyte-binding linear labeled oligonucleotide and a capture polymer under conditions whereby a complex is formed comprising the analyte, analyte-binding linear labeled oligonucleotide and capture polymer, wherein: (i)

the analyte-binding linear labeled oligonucleotide comprises (a) a sequence that is hybridizable to the analyte and (b) two or more units of label each attached directly to the oligonucleotide; (ii) the capture polymer comprises a first portion that is hybridizable to the analyte and and a second portion comprising a material (preferably but not necessarily a non-nucleic acid material) that is not substantially hybridizable to nucleic acid; and (B) detecting or quantitating the complex of step (A); whereby detection or quantitation of the complex of step (A) is indicative of presence or quantity of the nucleic acid analyte in the sample.

In one aspect, the invention provides a method for detecting or quantitating a nucleic acid analyte in a sample, said method comprising: (A) contacting said sample with an analyte-binding oligonucleotide, a labeled oligonucleotide, a capture polymer and a linear stem oligonucleotide under conditions whereby a complex is formed comprising the analyte, analyte-binding oligonucleotide, labeled oligonucleotide, capture polymer and linear stem oligonucleotide, wherein: (i) the analyte-binding oligonucleotide comprises (a) a sequence that is hybridizable to the analyte and (b) a sequence that is hybridizable to the stem oligonucleotide; (ii) the linear stem oligonucleotide comprises (a) a sequence that is hybridizable to the analyte-binding oligonucleotide and (b) a sequence that is directly or indirectly hybridizable to the labeled oligonucleotide; (iii) the labeled oligonucleotide comprises (a) a sequence that is directly or indirectly hybridizable to the stem oligonucleotide and (b) a label capable of directly or indirectly generating a detectable signal; (iv) the capture polymer comprises a nucleic acid sequence that is hybridizable to the analyte and further comprises at least one modified nucleotide that enhances strength of hybridization of the polymer to the analyte; (B) detecting or quantitating the complex of step (A); whereby detection or quantitation of the complex of step (A) is indicative of presence or quantity of the nucleic acid analyte in the sample. In one embodiment, the labeled oligonucleotide is a linear oligonucleotide. In some embodiments, the labeled oligonucleotide is a linear labeled oligonucleotide that comprises two or more units of label each attached directly to the oligonucleotide. In one embodiment, the linear stem oligonucleotide comprises a sequence that is directly hybridizable to the labeled oligonucleotide and the labeled oligonucleotide comprises a sequence that is directly hybridizable to the stem oligonucleotide.

In one aspect, the invention provides a method for detecting or quantitating a nucleic acid analyte in a sample, said method comprising: (A) contacting said sample with an analyte-binding oligonucleotide, a linear labeled oligonucleotide and a capture polymer under conditions whereby a complex is formed comprising the analyte, analyte-binding oligonucleotide, linear labeled oligonucleotide and capture polymer, wherein: (i) the analyte-binding oligonucleotide comprises (a) a sequence that is hybridizable to the analyte and (b) a sequence that is hybridizable to the linear labeled oligonucleotide; (ii) the linear labeled oligonucleotide comprises (a) two or more units of label each attached directly to the oligonucleotide and (b) a sequence that is hybridizable to the analyte-binding oligonucleotide; (iii) the capture polymer comprises a nucleic acid sequence that is hybridizable to the analyte and further comprises at least one modified nucleotide that enhances strength of hybridization of the polymer to the analyte; and (B) detecting or quantitating the complex of step (A); whereby detection or quantitation of the complex of step (A) is indicative of presence or quantity of the nucleic acid analyte in the sample.

In one aspect, the invention provides a method for detecting or quantitating a nucleic acid analyte in a sample, said method comprising: (A) contacting said sample with an analyte-binding linear labeled oligonucleotide and a capture polymer under conditions whereby a complex is formed comprising the analyte, analyte-binding linear labeled oligonucleotide and capture polymer, wherein: (i) the analyte-binding linear labeled oligonucleotide comprises (a) a sequence that is hybridizable to the analyte and (b) two or more units of label each attached directly to the oligonucleotide; (ii) the capture polymer comprises a nucleic acid sequence that is hybridizable to the analyte and further comprises at least one modified nucleotide that enhances strength of hybridization of the polymer to the analyte; (B) detecting or quantitating the complex of step (A); whereby detection or quantitation of the complex of step (A) is indicative of presence or quantity of the nucleic acid analyte in the sample.

In another aspect, the invention provides a method for detecting or quantitating a nucleic acid analyte in a sample, said method comprising: (A) contacting said sample with an analyte-binding oligonucleotide, a linear labeled oligonucleotide, a capture polymer and a stem oligonucleotide under conditions whereby a complex is formed comprising the analyte, analyte-binding oligonucleotide, labeled oligonucleotide, capture polymer and linear stem oligonucleotide, wherein: (i) the analyte-binding oligonucleotide comprises (a) a sequence that is hybridizable to the analyte and (b) a sequence that is hybridizable to the stem oligonucleotide; (ii) the stem oligonucleotide comprises (a) a sequence that is hybridizable to the analyte-binding oligonucleotide and (b) a sequence that is directly or indirectly hybridizable to the linear labeled oligonucleotide; (iii) the labeled oligonucleotide comprises (a) a sequence that is directly or indirectly hybridizable to the stem oligonucleotide and (b) a label capable of directly or indirectly generating a detectable signal; (iv) the capture polymer comprises a first portion that is hybridizable to the analyte, said first portion comprising at least one modified nucleotide that enhances strength of hybridization of the polymer to the analyte, and a second portion comprising a material (preferably but not necessarily a non-nucleic acid material) that is not substantially hybridizable to nucleic acid; and (B) detecting or quantitating the complex of step (A); whereby detection or quantitation of the complex of step (A) is indicative of presence or quantity of the nucleic acid analyte in the sample. In one embodiment, the labeled oligonucleotide is a linear oligonucleotide. In some embodiments, the labeled oligonucleotide is a linear labeled oligonucleotide that comprises two or more units of label each attached directly to the oligonucleotide. In one embodiment, the linear stem oligonucleotide comprises a sequence that is directly hybridizable to the labeled oligonucleotide and the labeled oligonucleotide comprises a sequence that is directly hybridizable to the stem oligonucleotide.

In another aspect, the invention provides a method for detecting or quantitating a nucleic acid analyte in a sample, said method comprising: (A) contacting said sample with an analyte-binding oligonucleotide, a linear labeled oligonucleotide and a capture polymer under conditions whereby a complex is formed comprising the analyte, analyte-binding oligonucleotide, linear labeled oligonucleotide and capture polymer, wherein: (i) the analyte-binding oligonucleotide comprises (a) a sequence that is hybridizable to the analyte and (b) a sequence that is hybridizable to the linear labeled oligonucleotide; (ii) the linear labeled oligonucleotide comprises (a) two or more units of label each attached directly to the oligonucleotide and (b) a sequence that is hybridizable to the analyte-binding oligonucleotide; (iii) the capture polymer comprises a first portion that is hybridizable to the analyte, said first portion comprising at least one modified nucleotide that enhances strength of hybridization of the polymer to the analyte, and a second portion comprising a material (preferably but not necessarily a non-nucleic acid material) that is not substantially hybridizable to nucleic acid; (b) detecting or quantitating the complex of step (A); whereby detection or quantitation of the complex of step (A) is indicative of presence or quantity of the nucleic acid analyte in the sample.

In yet another aspect, the invention provides a method for detecting or quantitating a nucleic acid analyte in a sample, said method comprising: (A) contacting said sample with an analyte-binding linear labeled oligonucleotide and a capture polymer under conditions whereby a complex is formed comprising the analyte, analyte-binding linear labeled oligonucleotide and capture polymer, wherein: (i) the analyte-binding linear labeled oligonucleotide comprises (a) a sequence that is hybridizable to the analyte and (b) two or more units of label each attached directly to the oligonucleotide; (ii) the capture polymer comprises a first portion that is hybridizable to the analyte, said first portion comprising at least one modified nucleotide that enhances strength of hybridization of the polymer to the analyte, and a second portion comprising a material (preferably but not necessarily a non-nucleic acid material) that is not substantially hybridizable to nucleic acid; and (B) detecting or quantitating the complex of step (A); whereby detection or quantitation of the complex of step (A) is indicative of presence or quantity of the nucleic acid analyte in the sample.

In another aspect, the invention provides a method for detecting or quantitating a nucleic acid analyte in a sample, said method comprising: (A) contacting said sample with a capture polymer under conditions whereby a complex is formed comprising the analyte and capture polymer, wherein the capture polymer comprises a sequence that is hybridizable to the analyte, and wherein said sequence comprises at least one modified nucleotide that enhances strength of hybridization of the polymer to the analyte; and (B) detecting or quantitating the complex of step (A); whereby detection or quantitation of the complex of step (A) is indicative of presence or quantity of the nucleic acid analyte in the sample. The analyte may be directly or indirectly labeled.

In yet another aspect, the invention provides a method for detecting or quantitating a nucleic acid analyte in a sample, said method comprising: (A) contacting said sample with a capture polymer under conditions whereby a complex is formed comprising the analyte and capture polymer, wherein the capture polymer comprises a first portion that is hybridizable to the analyte, and a second portion comprising a material (preferably but not necessarily a non-nucleic acid material) that is not substantially hybridizable to nucleic acid; and (B) detecting or quantitating the complex of step (A); whereby detection or quantitation of the complex of step (A) is indicative of presence or quantity of the nucleic acid analyte in the sample. The analyte may be directly or indirectly labeled.

In yet another aspect, the invention provides a method for detecting or quantitating a nucleic acid analyte in a sample, said method comprising: (A) contacting said sample with a capture polymer under conditions whereby a complex is formed comprising the analyte and capture polymer, wherein the capture polymer comprises a first portion that is hybridizable to the analyte, said first portion comprising at least one modified nucleotide that enhances strength of hybridization of the polymer to the analyte, and a second portion comprising a material (preferably but not necessarily a non-nucleic acid material) that is not substantially hybridizable to nucleic acid; and (B) detecting or quantitating the complex of step (A); whereby detection or quantitation of the complex of step (A) is indicative of presence or quantity of the nucleic acid analyte in the sample. The analyte may be directly or indirectly labeled.

In some embodiments of the methods described herein, two tandem units of label of a linear labeled oligonucleotide are separated by at least about 1, 3 or 5 nucleotides. In some embodiments, two tandem units of label of a linear labeled oligonucleotide are separated by from about 1 to about 12 nucleotides. In certain embodiments, two tandem units of label of a linear labeled oligonucleotide are separated by from about 3 to about 10 nucleotides. In some embodiments, two tandem units of label of a linear labeled oligonucleotide are separated by from about 5 to about 8 nucleotides. In some embodiments of linear labeled oligonucleotides of the invention, a label is attached by covalent bond to the linear labeled oligonucleotide.

Any of a variety of labels capable of directly or indirectly generating detectable signal may be used. In one embodiment, a label on a labeled oligonucleotide is selected from the group consisting of an antigen, a member of a specific binding pair, a fluorescent dye and a member of a reporter-quencher pair. In some embodiments, an antigen label is selected from the group consisting of digoxigenin, biotin and fluorescein isothiocyanate. In some embodiments, a specific binding pair label is selected from the group consisting of a receptor-ligand pair and an enzyme-substrate pair. In some embodiments, a fluorescent dye label is fluorescein isothiocyanate, rhodamine or Texas Red. In some embodiments, a reporter-quencher pair comprises a dye or dyes capable of fluorescent resonance energy transfer.

Labeled oligonucleotides can be detected by any means appropriate to the label type. Thus, in some embodiments, a labeled oligonucleotide (such as a linear labeled oligonucleotide of the invention) is detected by contacting the labeled oligonucleotide (which is generally in a complex comprising analyte and other oligonucleotides/polymers as would be expected according to methods of the invention) with a compound that binds to the labels of the labeled oligonucleotide, wherein said compound is capable of directly or indirectly generating a detectable signal.

In methods of the invention, the sequence of an analyte-binding oligonucleotide that is hybridizable to an analyte may be completely complementary with respect to the sequence of the analyte to which it is hybridizable, or of less than complete complementarity with respect to the sequence of the analyte to which it is hybridizable so long as hybridization between the oligonucleotide and analyte can occur under reaction conditions. Thus, in some embodiments, the sequence is of at least 50%, at least about 60%, at least about 75%, at least about 85%, at least about 95%, at least about 98%, at least about 99%, or 100% (i.e., complete) complementarity to the sequence of the analyte to which it is hybridizable.

Capture polymers for use in methods of the invention may be provided in any of a number of forms. In some embodiments, capture polymers are provided as an array, such as on microwell plates (for example, 96-well or 384-well plates). In some embodiments, capture polymers are provided as microarrays, such as on glass or plastic slides.

In some embodiments of methods of the invention wherein a capture polymer comprising a first portion that is hybridizable to the analyte and a second portion comprising a material that is not substantially hybridizable to nucleic acid is used, the second portion of the capture polymer comprises substantially all of the length of the capture polymer other than the first portion. In some embodiments, at least 10% of the length of the capture polymer is a material that is not substantially hybridizable to nucleic acid. In certain embodiments, at least 25% of the length of the capture polymer is a material that is not substantially hybridizable to nucleic acid. In some embodiments, at least 40% of the length of the capture polymer is a material that is not substantially hybridizable to nucleic acid. In some embodiments, at least 50% of the length of the capture polymer is a material that is not substantially hybridizable to nucleic acid. In another embodiment, from about 5% to about 90% of the length of the capture polymer is a material that is not substantially hybridizable to nucleic acid. In yet another embodiment from about 10% to about 70% of the length of the capture polymer is a material that is not substantially hybridizable to nucleic acid. In one embodiment, from about 20% to about 50% of the length of the capture polymer is a material that is not substantially hybridizable to nucleic acid.

In embodiments wherein a capture polymer comprises material that is not substantially hybridizable to nucleic acid, said material may be any material known in the art and/or empirically shown to possess this characteristic and that does not substantially interfere with analyte detection and quantitation under reaction conditions. In some embodiments, the material is a non-nucleic acid material. Suitable materials include inert carbon, which may be provided in the form of, for example, ethylene glycol having the chemical structure 18-O-Dimethoxytritylhexaethyleneglycol, 1-[(2-cyanoethyl)-(N,N-diisopropyl)]-phosphoramidite.

In any method of the invention, the capture polymer may comprise a spacer component. In some embodiments, the spacer component comprises at least one C18 spacer. In other embodiments, the spacer component comprises at least three C18 spacers. In other embodiments, the spacer component comprises at least four C18 spacers. In some embodiments, the spacer component comprises from about 1 to about 8 C18 spacers. In certain embodiments, the spacer component comprises from about 3 to about 6 C18 spacers. In some embodiments of methods of the invention, the spacer component of a capture polymer is the material that is not substantially hybridizable to nucleic acid of the second portion of the capture polymer as described herein (for e.g., in the preceding paragraph).

In one embodiment of methods of the invention wherein a capture polymer comprising at least one modified nucleotide that enhances hybridization strength is used, the capture polymer comprises at least about 3 said modified nucleotides. In another embodiment, the capture polymer comprises at least about 5 said modified nucleotides. In one embodiment, at least about 10 percent of the total number of nucleotides in the capture polymer are said modified nucleotide. In another embodiment, at least about 20 percent of the total number of nucleotides in the capture polymer are said modified nucleotide. In yet another embodiment, at least about 30 percent of the total number of nucleotides in the capture polymer are said modified nucleotide. In another embodiment, at least about 40 percent of the total number of nucleotides in the capture polymer are said modified nucleotide. In still another embodiment, at least about 50 percent of the total number of nucleotides in the capture polymer are said modified nucleotide. In one embodiment, from about 10 to about 50 percent of the total number of nucleotides in the capture polymer are said modified nucleotide.

In embodiments wherein a capture polymer comprises at least one modified nucleotide that enhances hybridization strength, said modified nucleotide may be any known in the art and/or empirically shown to possess this characteristic and that does not substantially interfere with analyte detection and quantitation under reaction conditions. Such modified nucleotides include modified ribonucleotides such as 2′-O-methoxy-RNA or derivative thereof, peptide nucleic acid and locked nucleic acid.

In some embodiments wherein a capture polymer comprises at least one modified nucleotide that enhances hybridization strength, at least one modified nucleotide is located in the 5′ region of the sequence that is hybridizable to analyte. In other embodiments, at least one modified nucleotide is located in the 3′ region of the sequence that is hybridizable to analyte. In some embodiments, at least one said modified nucleotide is located in each of the 5′ and 3′ regions of the sequence that is hybridizable to analyte.

Capture polymers of methods of the invention may be directly or indirectly attached to a support, which may be, for example, a semi-solid or solid material. In one embodiment, a capture polymer is indirectly attached to a support. In one embodiment, a capture polymer is hybridized to an extender oligonucleotide that is attached to a support. In another embodiment, a capture polymer is directly attached to a support.

Blocker oligonucleotides may also be included in methods of the invention. Accordingly, in some embodiments, methods of the invention further comprise contacting a sample with a blocker oligonucleotide, wherein the blocker oligonucleotide comprises a sequence that reduces non-specific binding or hybridization, for example non-specific binding or hybridization between analyte, oligonucleotides and/or capture polymers. In one embodiment of methods of the invention wherein a capture polymer is indirectly attached to support, the reaction mixture comprises a blocker oligonucleotide.

The various steps of methods of the invention do not necessarily have to be performed simultaneously or in a continual/continuous series. For example, the detection or quantitation process may be carried out up to the point of complex formation between analyte and the relevant component oligonucleotide(s) and/or polymers, while detection/quantitation of the complex (i.e., the analyte) is carried out at a later time. In some embodiments of methods of the invention, analyte is detected or quantitated by detecting or quantitating complex comprising the analyte (for example, the complex of step (A) or (a) in the various methods described above) present on a solid or semi-solid support. In some embodiments of methods of the invention, the methods further comprise, after step (A) or (a), washing complex comprising analyte (for example, the complex of step (A) or (a) in the various methods described above) (which may be present on a solid or semi-solid support) to remove unbound sample and/or unhybridized oligonucleotide and capture polymer.

Methods of the invention are capable of detecting and quantitating any of a variety of forms of nucleic acid analyte. For example, a nucleic acid analyte may be in any form selected from the group consisting of RNA, DNA, RNA/DNA hybrid and nucleic acid-protein complex. In some embodiments, a nucleic acid analyte comprises a sequence encoding part or all of a polypeptide selected from the group consisting of growth hormone, insulin-like growth factors, human growth hormone, N-methionyl human growth hormone, bovine growth hormone, parathyroid hormone, thyroxine, insulin, proinsulin, relaxin, prorelaxin, glycoprotein hormones, follicle stimulating hormone (FSH), thyroid stimulating hormone (TSH), leutinizing hormone (LH), hematopoietic growth factor, vesicular endothelial growth factor (VEGF), hepatic growth factor, fibroblast growth factor, prolactin, placental lactogen, tumor necrosis factor-alpha, tumor necrosis factor-beta, mullerian-inhibiting substance, mouse gonadotropin-associated peptide, inhibin, activin, vascular endothelial growth factor, integrin, nerve growth factors (NGFs), NGF-beta, platelet-growth factor, transforming growth factors (TGFs), TGF-alpha, TGF-beta, insulin-like growth factor-I, insulin-like growth factor-II, erythropoietin (EPO), osteoinductive factors, interferons, interferon-alpha, interferon-beta, interferon-gamma, colony stimulating factors (CSFs), macrophage-CSF (M-CSF), granulocyte-macrophage-CSF (GM-CSF), granulocyte-CSF (G-CSF), thrombopoietin (TPO), interleukins (ILs), IL-1, IL-1alpha, IL-2, IL-3, IL-4, IL-5, IL-6, IL-7, IL-8, IL-9, IL-11, IL-12, LIF, SCF, neurturin (NTN), kit-ligand (KL), HER2, human Fc, human heavy and light chains (constant region), KDR, nitric oxide synthase (NOS) and angiotensin converting enzyme (ACE). A sample suspected or known to contain an analyte may be in any one of a number of forms and of any one of a number of sources. In one embodiment, a sample is selected from the group consisting of blood, serum, sputum, urine, semen, cerebrospinal fluid, bronchial aspirate, organ tissue, cell lysate and cell culture medium.

The invention also provides the oligonucleotides and capture polymers as described herein. These oligonucleotides and capture polymers can be provided in any form. For example, capture polymers of the invention can be adapted for use in a variety of nucleic acid capture assays. Capture polymers can, for example, conveniently be provided as arrays or microarrays. Accordingly, the invention also provides an array or microarray of a capture polymer of the invention attached to a solid or semi-solid support. In some embodiments, an array comprises capture polymers provided on a 96-well plate. In another embodiment, an array comprises capture polymers provided on a 384-well plate. In one embodiment, a microarray comprises capture polymers provided on a glass or plastic slide. Details of arrays and microarrays are provided herein.

The invention also provides compositions, kits and articles of manufacture comprising oligonucleotides and capture polymers of the invention, either singly or in any combination. Reaction mixtures, reaction complexes and products related to methods of the invention are also provided. Details of these compositions, kits and articles of manufacture are provided herein.

### BRIEF DESCRIPTION OF THE DRAWINGS

FIG. 1 is a schematic depiction of one embodiment of a method of the invention wherein linear stem oligonucleotide, analyte-binding oligonucleotide and labeled oligonucleotide are used.

FIG. 2 is a schematic depiction of one embodiment of a method of the invention wherein linear labeled oligonucleotide and analyte-binding oligonucleotide are used.

FIG. 3 is a schematic depiction of one embodiment of a method of the invention wherein an analyte-binding linear labeled oligonucleotide is used and the capture polymer is indirectly attached to a support.

FIG. 4 is a schematic depiction of one embodiment of a method of the invention wherein an analyte-binding labeled oligonucleotide is used and the capture polymer is directly attached to a support.

FIG. 5 is a schematic depiction of one embodiment of a method of the invention wherein a capture polymer comprising a material (depicted as 3′-ethylene glycol scaffolding) that is not substantially hybridizable to nucleic acid is used.

FIG. 6 is a schematic depiction of one embodiment of a method of the invention wherein a capture polymer comprising (a) a material (depicted as 3′-ethylene glycol scaffolding) that is not substantially hybridizable to nucleic acid and (b) modified nucleotides that enhance hybridization strength is used.

FIGS. 7A-D depict sequences of capture polymers and component oligonucleotides (including analyte-binding oligonucleotides) used in Example 1 to detect human fetal (gamma), adult (beta), epsilon and delta hemoglobin RNA.

FIG. 8 depicts the design of an experiment to determine effects of using a plurality of species of capture polymers per reaction and the data obtained in the experiment.

FIG. 9 depicts data showing effects of direct/indirect attachment of capture polymers and effects of modifying capture polymers.

FIG. 10 depicts sequences of capture polymers and analyte-binding linear labeled oligonucleotides used in Examples 2 & 3 to detect human fetal (gamma) hemoglobin RNA.

FIG. 11 depicts data showing the effects of modifying capture polymers to include a material that is not substantially hybridizable to nucleic acid and modified nucleotides that enhance hybridization strength. Signal/noise ratios using these modified capture polymers are compared to those obtained with unmodified capture polymers that are directly or indirectly attached to a support.

FIG. 12 depicts data showing effects of source of alkaline phosphatase substrate (A1 & B1); choice of signal reader (A2 & B2); and microplate format (A3 & B3). Light bars represent data obtained by the “indirect & unmodified” method (see Example 3). Dark bars represent data obtained by the “direct & modified” method (see Example 3).

FIGS. 13A & B depict sequences of capture polymers, analyte-binding oligonucleotides and labeled oligonucleotides used to detect human Fc mRNA in Example 4.

FIG. 14 depicts data from Example 4. Light bars represent data obtained with an analyte-binding oligonucleotide that was indirectly labeled through hybridization with a linear labeled oligonucleotide. Dark bars represent data obtained with an analyte-binding oligonucleotide that was directly labeled.

FIG. 15 depicts data from detection and quantitation of labeled human fetal hemoglobin cDNAs in a DNA array format.

FIG. 16 schematically illustrates an embodiment of a cell line development process.

FIG. 17 sets forth sequences for primers and probes used in Taqman analysis of human Fc and GAPDH (as control) as described in Example 6.

FIGS. 18A-D depict data demonstrating applicability of methods of the invention to production cell clone screening by comparing quantitation data obtained by methods of the invention with data obtained by a conventional assay. The term “NACA” in the figures refer to a method of the invention as described in Example 6.

### MODES FOR CARRYING OUT THE INVENTION

The invention provides methods and compositions for detecting and quantitating nucleic acid analytes. The methods generally comprise using modified signaling oligonucleotides and/or capture polymers that individually or in combination increase signal to noise ratio of analyte detection and quantitation through improvements in specificity of analyte hybridization and sensitivity of analyte detection. Contrary to methods of the art which rely on components that are complex (due largely to a need to significantly enhance signal over a background of a substantial level of noise), the design of components of methods of the invention is distinctly simpler while providing at least equal analyte detection/quantitation specificity and sensitivity. These methods have the added advantage of ease of development and use because of the simple features of the methods generally and the component oligonucleotides specifically (for example, as seen in the simple linear design of labeled/signaling oligonucleotides, and, where desired or necessary, the ability to directly attach capture polymers to supports rather than through an extender oligonucleotide). Furthermore, the invention enables the quick development of any hybridization assays combining a multi-probe direct capture system and a multi-probe signaling system.

As a general summary, the invention works as follows: a sample suspected of containing a nucleic acid analyte is contacted with an analyte-binding oligonucleotide and a capture polymer (as described in greater detail herein) under conditions suitable for hybridization of the analyte-binding oligonucleotide and the capture polymer to the analyte. The invention provides various embodiments of the capture polymer that can be used in methods of the invention. Generally, the capture polymer can be directly or indirectly attached to a support (which can be, for example, a solid or semi-solid material), thus immobilizing any complex that comprises the capture polymer. The hybridization of these molecules results in a complex that can then be detected using a variety of methods known in the art, some of which are described herein. In one aspect of the invention, formation of the complex is detected by including a linear labeled oligonucleotide in the methods of the invention. The linear labeled oligonucleotide (which is described in greater detail below) is capable of hybridizing to the analyte-binding oligonucleotide. Thus, detection of the linear labeled oligonucleotide (for example through detection of the labels present on the oligonucleotide) on the support to which the capture polymer is attached provides an indication of the presence of a complex comprising the analyte, which in turn indicates presence of the analyte in the sample. Using techniques known in the art, the amount of the linear labeled oligonucleotide that is detected can be quantitated, for example, by comparing to a reference sample containing a known quantity of analyte.

In one aspect, the invention provides microarrays comprising a capture polymer of the invention attached directly or indirectly to a solid or semi-solid support. In some embodiments, the microarrays of the invention comprise a single species of capture polymer (i.e., the capture polymers comprise identical or substantially identical analyte-binding nucleic acid sequences) in each discrete spot on the microarray. In other embodiments, the microarrays of the invention comprise a plurality (i.e., two or more) species of capture polymers (i.e., the capture polymers comprise different analyte-binding nucleic acid sequences) in each discrete spot on the microarray.

The methods of the invention are also useful for multiplex analysis of nucleic acid analytes. That is to say, by using a plurality of linear labeled oligonucleotides (each species of oligonucleotide having a different label), various target nucleic acid sequences may be detected in a single reaction mixture. The various target sequences may be part of a single piece of nucleic acid, or may represent specific sequences of various nucleic acid targets, which may be present in a single test sample. For example, methods of the invention can detect, in a single reaction mixture, the presence of various pathogens in a single biological sample, or various polymorphic sites in a single genomic DNA sample.

The methods of the invention can be used in a number of applications as would be evident to one skilled in the art, some of which are described herein. For example, they can be used for diagnostic applications, such as in detecting or quantitating the expression of specific gene analytes and in detecting nucleic acid mutations of interest (such as single nucleotide polymorphisms). Because of the simplicity and flexibility of various components of the methods, the methods of the invention are particularly amenable to automation and adaptation in microarray form, which in turn provides greater high throughput potential.

General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988).

Oligonucleoitdes, polynucleotides and polymers employed or described in the present invention can be generated using standard techniques known in the art.

Definitions

“Analyte,” as used herein, refers to a nucleic acid sequence of which the detection and/or quantitation is desired using methods of the invention.

“Polynucleotide,” or “nucleic acid,” as used interchangeably herein, refer to polymers of nucleotides of any length, and include, but are not limited to, DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, .alpha.-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR.sub.2 (“amidate”), P(O)R, P(O)OR′, CO or CH.sub.2 (“formacetal”), in which each R or R′ is independently H or substituted or unsubstituted alkyl (1-20 C.) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA. It would be evident to one skilled in the art that forms of the polynucleotides as described in this paragraph and elsewhere herein are suitable so long as they do not substantially inhibit analyte detection or quantitation by methods of the invention.

“Oligonucleotide,” as used herein, generally refers to short, generally single stranded, generally synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides.

A “blocker oligonucleotide,” as used herein, refers to an oligonucleotide that when present in a reaction mixture reduces non-specific hybridization among components of the reaction mixture. Preferably, a blocker oligonucleotide comprises a sequence that is hybridizable to a sequence of an analyte to which none of the other components (for e.g., capture polymer, analyte-binding oligonucleotide, stem oligonucleotide, labeled oligonucleotide, linker/extender oligonucleotide) in a reaction mixture is intended to be hybridizable. For example, a blocker oligonucleotide may be used to hybridize to sequences in an analyte to which a capture polymer and/or analyte-binding oligonucleotide is not intended to be hybridizable, in particular when a capture polymer is indirectly attached to a support (i.e., it is hybridized to a linker oligonucleotide/polymer that is directly attached to the support). Analytes can frequently nonspecifically bind to linker (extender) oligonucleotides, in particular when the capture polymer is not directly attached to a support. Use of a blocker oligonucleotide may reduce background noise by, for example, binding to sequences of the analyte that may be involved in such nonspecific binding.

The phrase “a sequence that is hybridizable” and variations thereof, as used herein, refers to the ability of a sequence to form a duplex of variable strength depending on its melting temperature (Tₘ), the base complementarity with the target sequence, as well as the reaction conditions. The meaning of this phrase is known to persons skilled in the art.

The phrase “not substantially hybridizable”, as used herein, refers to a lack of ability of a sequence or material to form a duplex with another nucleic acid sequence. For example, generally, a sequence or material is not substantially hybridizable to another nucleic acid sequence if, under a particular set of reaction conditions, less than preferably about 5%, preferably about 3%, preferably about 1%, preferably about 0.5% of total complexes prior to detection of label comprises a duplex of said another nucleic acid sequence and the sequence or material that is not substantially hybridizable to said another nucleic acid sequence. Generally, a first sequence is not hybridizable to a second sequence if the duplex comprising the first and second sequences has a melting temperature that is less than preferably about 10° C. or about 5° C. above the temperature condition of the detection reaction.

“Non-specific hybridization”, as used herein, refers to the interaction of an oligonucleotide to a nucleic acid sequence different from the sequence to which the oligonucleotide is designed to be hybridizable. Nonspecific hybridization may trigger erroneous results by either increasing or decreasing assay signal without correlation to the presence or absence of an analyte.

“Non-specific binding,” as used herein, refers to direct or indirect binding of molecule (for e.g., nucleic acid or peptidic structure) to another molecule (such as solid or semi-solid support) that does not involve specific hybridization. Non-specific binding may trigger erroneous results by either increasing or decreasing assay signal without correlation to the presence or absence of an analyte. In certain contexts that would be evident to one skilled in the art, the phrases “non-specific binding” and “non-specific hybridization” are interchangeable.

“Percent (%) nucleic acid sequence identity” with respect to a sequence of an analyte or non-analyte is defined as the percentage of nucleotides in an analyte that are identical with the nucleotides in a sequence of another nucleic acid molecule (such as a potentially interfering non-analyte), after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % nucleic acid sequence identity values are obtained by using the sequence comparison computer program ALIGN-2 (Genentech, Inc., South San Francisco, Calif., USA).

To “inhibit” is to decrease or reduce an activity, function, and/or amount as compared to a reference.

A “complex” is an assembly of components. A complex may or may not be stable and may be directly or indirectly detected. For example, as is described herein, given certain components of a reaction, and the type of product(s) of the reaction, existence of a complex can be inferred.

A “portion” or “region,” used interchangeably herein, of a polynucleotide or oligonucleotide is a contiguous sequence of 2 or more bases. In other embodiments, a region or portion is at least about any of 3, 5, 10, 15, 20, 25 contiguous nucleotides.

The term “3′” generally refers to a region or position in a polynucleotide or oligonucleotide 3′ (downstream) from another region or position in the same polynucleotide or oligonucleotide.

The term “5′” generally refers to a region or position in a polynucleotide or oligonucleotide 5′ (upstream) from another region or position in the same polynucleotide or oligonucleotide.

A “reaction mixture” is an assemblage of components, which, under suitable conditions, react to form a complex (which may be an intermediate) and/or a product(s).

“A”, “an” and “the”, and the like, unless otherwise indicated include plural forms.

“Comprising” means including.

Conditions that “allow” an event to occur or conditions that are “suitable” for an event to occur, such as hybridization, detection, complex formation and the like, or “suitable” conditions are conditions that do not prevent such events from occurring. Thus, these conditions permit, enhance, facilitate, and/or are conducive to the event. Such conditions, known in the art and described herein, depend upon, for example, the nature of the nucleotide sequence, temperature, and buffer conditions. These conditions also depend on what event is desired, such as hybridization, detection or quantitation.

“Microarray” and “array,” as used interchangeably herein, refer to an arrangement of a collection of nucleotide sequences in a centralized location. Arrays can be on a solid substrate, such as glass or plastic slides or microtiter plates (for example, 96, 384, 1536-well plates), or on a semi-solid substrate, such as nitrocellulose membrane. The nucleotide sequences can be DNA, RNA, or any permutations thereof.

“Detection” includes any means of detecting, including direct and indirect detection. Detection techniques are known in the art, some of which are described herein.

Methods of the Invention

The following are examples of the detection and quantitation methods of the invention. It is understood that various other embodiments may be practiced, given the general description provided above. It is also understood that detection and quantitation can be separate end goals. For example, in some instances, a practitioner may only wish to detect the presence of an analyte in a sample, without quantitating the analyte. Methods of the invention can be used in any of these instances.

Methods of Detection and Quantitation Using a Stem Oligonucleotide

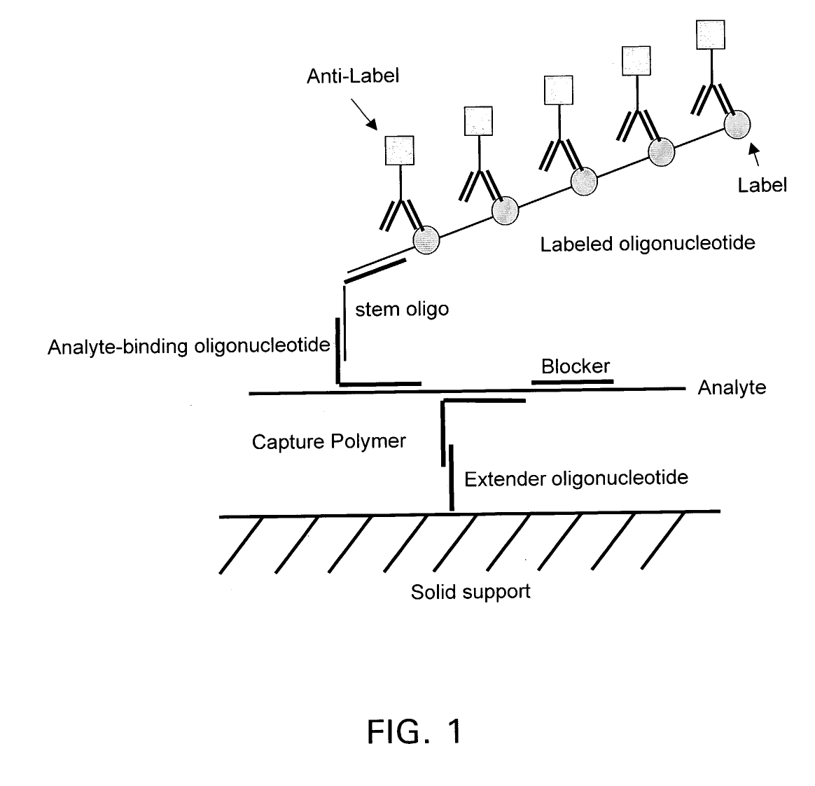

In one aspect, the invention provides methods of detection and quantitation wherein a stem oligonucleotide links a signaling system (such as a labeled oligonucleotide), directly or indirectly to a complex comprising analyte, stem oligonucleotide (which is preferably linear), capture polymer and analyte-binding oligonucleotide. Binding of the signaling system to the complex, directly or indirectly, through the stem oligonucleotide provides a means of detecting the formation of the complex. Formation of the complex is indicative of presence (and amount) of analyte in a sample. In one embodiment, one example of which is illustrated in FIG. 1, the analyte-binding oligonucleotide comprises (a) a sequence that is hybridizable to the analyte and (b) a sequence that is hybridizable, directly or indirectly, to a stem oligonucleotide (which is preferably a linear oligonucleotide). The stem oligonucleotide (which is preferably linear) comprises (a) a sequence that is hybridizable, directly or indirectly, to the analyte-binding oligonucleotide and (b) a sequence that is hybridizable, directly or indirectly, to a labeled oligonucleotide (for example, a linear labeled oligonucleotide of the invention). A linear labeled oligonucleotide of the invention comprises (a) two or more units of label each attached directly to the oligonucleotide and (b) a sequence that is hybridizable, directly or indirectly, to the stem oligonucleotide. The capture polymer comprises a sequence that is directly or indirectly hybridizable to the analyte. A sample suspected of containing a nucleic acid analyte is contacted with an analyte-binding oligonucleotide, a labeled oligonucleotide, a stem oligonucleotide (which is preferably linear) and a capture polymer under conditions whereby, if the analyte is present in the sample, a complex comprising the analyte, the analyte-binding oligonucleotide, the labeled oligonucleotide, the stem oligonucleotide and the capture polymer is formed. In general, the capture polymer is directly or indirectly attached to a support, which is generally comprised of a solid or semi-solid material. Attachment of the capture polymer to the support may be prior to, during or following the reaction wherein the complex of interest is formed. A complex of interest that is formed would remain on the surface of the support when unbound sample and/or components (i.e., analyte-binding oligonucleotides, stem oligonucleotides, labeled oligonucleotides and capture polymers) are washed away.

The complex that remains on the support can be detected in any of a number of ways. Preferably, the complex is contacted with a label-detection compound that binds to the labels on the labeled oligonucleotide, wherein the label-detection compound is capable of directly or indirectly generating a detectable signal. In one example, a label may be a member of a specific binding pair, such as a receptor-ligand pair or antibody-antigen pair. For example, if the label is an antigen (such as digoxigenin), an antibody specific for the antigen can be used. The antibody can itself generate a detectable signal, for example, through a signal producing moiety attached to the antibody. The antibody can also generate a detectable signal indirectly, for example, through an enzyme attached to it, which enzyme is capable of catalyzing a reaction when contacted with a substrate to produce a detectable signal. Suitable enzymes include, but are not limited to, lacZ, horseradish peroxidase, alkaline phosphatase. Other specific binding pairs are known in the art, for example ligands that have natural anti-ligands, such as biotin, thyroxine and cortisol. Various signal producing moieties and combinations are well known in the art, some of which are described herein. In instances wherein signal amplification is not desired, the labels on the linear labeled oligonucleotide can be moieties that are capable of generating a detectable signal without being first contacted with a label-detection compound. Examples of such labels include fluorescein isothiocyanate, rhodamine, Texas Red, radioisotopes (e.g., ³H, ³⁵S, ³²P, ³³P, ¹²⁵I, ¹⁴C) and colorimetric labels (such as colloidal gold, colored glass or plastic (e.g., polystyrene, polypropylene, latex, etc.) beads).

A capture polymer, as used in methods of the invention, may be attached directly or indirectly to a solid or semi-solid support. Solid materials include, for example, glass and plastic. Semi-solid materials include, for example, gelatin compounds and nitrocellulose membrane. When a capture polymer is attached directly to a solid or semi-solid support, the attachment is preferably, but not necessarily, by covalent bonds. Methods of attaching a polymer, such as a polynucleotide or oligonucleotide, to a solid or semi-solid material are well known in the art. For example, quinone photochemistry, which is available commercially as DNA Immobilizer™ from EXIQON (Vedbaek, Denmark), can be used. Quinone photochemistry is particularly useful for covalently attaching a DNA-based capture polymer to a solid polymeric material such as plastic. In another example, biotinylated capture polymers can be attached to streptavidin-coated plastic or glass surface (for example, plates available commercially from Pierce (Cat. No. 15118)). Capture polymers may also be indirectly attached to a support through hybridization to an extender oligonucleotide that is directly attached to the support. Indirect attachment of capture polymers is a well-known technique in the art, as described in, for example, U.S. Pat. No. 5,635,352.

Methods of the invention can be used for multiplex analysis of analytes, wherein two or more analytes comprising different sequences are detected or quantitated in a single reaction mixture. In these embodiments, a plurality of species of analyte-binding oligonucleotides and labeled oligonucleotides are used. A plurality of species of analyte-binding oligonucleotides would comprise two or more species of analyte-binding oligonucleotides, each species comprising (a) a sequence that is specifically hybridizable to a specific analyte and (b) a sequence that is hybridizable, directly or indirectly, to a species of stem oligonucleotide. A plurality of species of labeled oligonucleotides would comprise two or more species of labeled oligonucleotides, each of which comprises a distinct label (relative to other species of the labeled oligonucleotide of the plurality). Each species of labeled oligonucleotide in the plurality further comprises a sequence that is hybridizable, directly or indirectly, to a species of stem oligonucleotide that is specific for a species of analyte-binding oligonucleotide. Thus, each species of labeled oligonucleotide corresponds to one species of analyte-binding oligonucleotide (and thus one specific analyte). Detection of the label associated with a particular species of labeled oligonucleotide would thus indicate the presence of the corresponding analyte.

A single analyte can be detected by methods of the invention utilizing a single species of capture polymers or a plurality of capture polymers in a single reaction mixture. A species of capture polymer is a capture polymer comprising a specific nucleic acid sequence that is hybridizable to an analyte. Thus, a plurality of capture polymers refers to two or more species of capture polymers, each of which comprising a different analyte-binding nucleic acid sequence. In some embodiments, each species of a plurality of capture polymer species comprises a different analyte-binding nucleic acid sequence, wherein each analyte-binding sequence is hybridizable to the same analyte. In these embodiments, a single analyte may be detected or quantitated using, in a single reaction mixture, preferably at least about 1, more preferably at least about 3, even more preferably at least about 5, still more preferably at least about 6 species of capture polymers. In some embodiments, a single analyte is detected or quantitated using, in a single reaction mixture, preferably from about 1 to about 10, more preferably from about 3 to about 8, even more preferably from about 5 to about 7 species of capture polymers. In other embodiments, each species of a plurality of capture polymer species comprises a different analyte-binding nucleic acid sequence, wherein each analyte-binding sequence is hybridizable to a different analyte (i.e., two or more analytes with non-identical nucleic acid sequences). These emobodiments are particularly useful in, for example, multiplex detection or quantitation of analytes.

Methods of the invention are capable of detection and quantitation of analytes present in a sample in a wide range of concentrations. In some embodiments, the concentration of analyte detectable and quantifiable by methods of the invention is preferably at least about 0.01 pg/mL, preferably at least about 70 pg/mL, preferably at least about 200 pg/mL, preferably at least about 2000 pg/mL, preferably at least about 5000 pg/mL, preferably at least about 20000 pg/mL, and preferably at least about 50000 pg/mL. In other embodiments, the concentration of analyte detectable and quantifiable by methods of the invention is preferably equal to or less than about 50000 pg/mL, preferably equal to or less than about 20000 pg/mL, preferably equal to or less than about 5000 pg/mL, preferably equal to or less than about 2000 pg/mL, preferably equal to or less than about 200 pg/mL, preferably equal to or less than about 70 pg/mL, and preferably equal to or less than about 0.01 pg/mL. In still other embodiments, the concentration of analyte detectable and quantifiable by methods of the invention is preferably from about 0.01 to about 100000 pg/mL, preferably from about 50 to about 75000 pg/mL, preferably from about 200 to about 50000 pg/mL, preferably from about 1000 to about 35000 pg/mL, and preferably from about 2000 to about 20000 pg/mL.

Methods of the invention provide high specificity of detection of nucleic acid analytes. In some embodiments, an analyte is detected with preferably less than about 5%, preferably less than about 2%, preferably less than about 1%, preferably less than about 0.5%, and preferably less than about 0.1% interference from a non-analyte nucleic acid molecule with high nucleotide sequence identity to the analyte, when the non-analyte nucleic acid molecule is present in a reaction mixture. Percent interference may be determined by techniques known in the art. For example, nucleic acids that are of high homology (but not identical) in sequence with respect to an analyte can be quantitated using an assay comprising oligonucleotides specific for detection of the analyte. Amount of “signal” obtained when the homologous nucleic acids are “detected” with oligonucleotides specific for the analyte, when expressed as a percentage of the signal obtained under similar reaction conditions for the analyte, would constitute the interference percentage. In some embodiments, the non-analyte nucleic acid molecule with high nucleotide sequence identity preferably has equal to or less than 85%, preferably equal to or less than 80%, preferably equal to or less than 70%, preferably equal to or less than 60% sequence identity with the analyte. In certain embodiments, the non-analyte nucleic acid molecule with high nucleotide sequence identity preferably has equal to or more than 60%, preferably equal to or more than 70%, preferably equal to or more than 80%, preferably equal to or more than 82%, preferably equal to or more than 90% sequence identity with the analyte. In other embodiments, the non-analyte nucleic acid molecule with high nucleotide sequence homology preferably has from about 50% to about 90%, preferably has from about 60% to about 85%, preferably from about 70% to about 85% sequence identity with the analyte.

Methods of Detection and Quantitation Using a Linear Labeled Oligonucleotide without a Stem Oligonucleotide

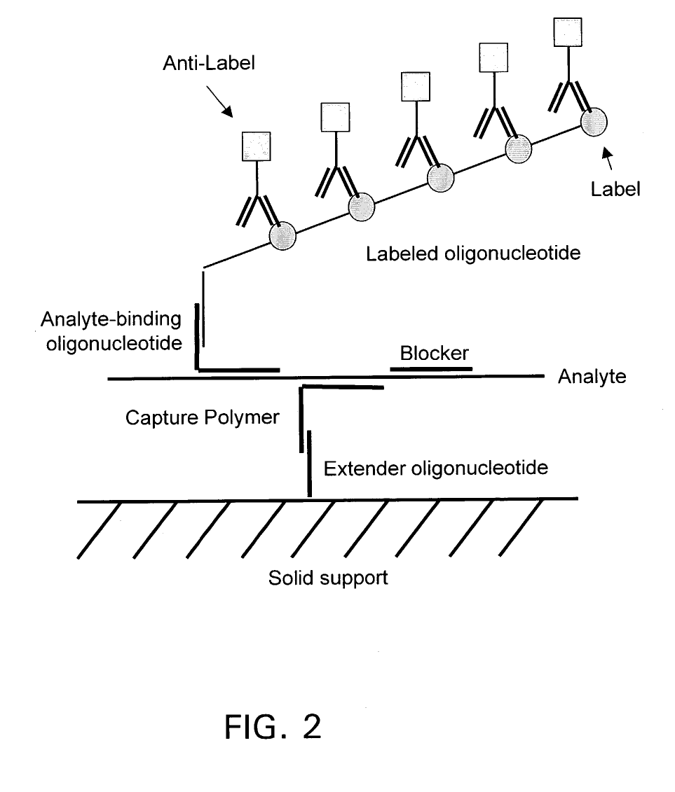

In one aspect, the invention provides methods of detection and quantitation wherein a linear labeled oligonucleotide of the invention is used to provide a means of detecting formation of a complex of analyte, capture polymer and analyte-binding oligonucleotide. In one embodiment, one example of which is illustrated in FIG. 2, the analyte-binding oligonucleotide comprises (a) a sequence that is hybridizable to the analyte and (b) a sequence that is hybridizable, directly or indirectly, to the linear labeled oligonucleotide. In this embodiment, the linear labeled oligonucleotide of the invention comprises (a) two or more units of label each attached directly to the oligonucleotide and (b) a sequence that is hybridizable, directly or indirectly, to the analyte-binding oligonucleotide. The capture polymer comprises a sequence that is directly or indirectly hybridizable to the analyte. A sample suspected of containing a nucleic acid analyte is contacted with an analyte-binding oligonucleotide, a linear labeled oligonucleotide, and a capture polymer under conditions whereby, if the analyte is present in the sample, a complex comprising the analyte, the analyte-binding oligonucleotide, the linear labeled oligonucleotide and the capture polymer is formed. In general, the capture polymer is directly or indirectly attached to a support, which is generally comprised of a solid or semi-solid material. Attachment of the capture polymer to the support may be prior to, during or following the reaction wherein the complex of interest is formed. A complex of interest that is formed would remain on the surface of the support when unbound sample and/or components (i.e., analyte-binding oligonucleotides, linear labeled oligonucleotides and capture polymers) are washed away.

The complex that remains on the support can be detected in any of a number of ways. Preferably, the complex is contacted with a label-detection compound that binds to the labels on the linear labeled oligonucleotide, wherein the label-detection compound is capable of directly or indirectly generating a detectable signal. In one example, a label may be a member of a specific binding pair, such as a receptor-ligand pair or antibody-antigen pair. For example, if the label is an antigen (such as digoxigenin), an antibody specific for the antigen can be used. The antibody can itself generate a detectable signal, for example, through a signal producing moiety attached to the antibody. The antibody can also generate a detectable signal indirectly, for example, through an enzyme attached to it, which enzyme is capable of catalyzing a reaction when contacted with a substrate to produce a detectable signal. Suitable enzymes include, but are not limited to, lacZ, horseradish peroxidase, alkaline-phosphatase. Other specific binding pairs are known in the art, for example ligands that have natural anti-ligands, such as biotin, thyroxine and cortisol. Various signal producing moieties and combinations are well known in the art, some of which are described herein. In instances wherein signal amplification is not desired, the labels on the linear labeled oligonucleotide can be moieties that are capable of generating a detectable signal without being first contacted with a label-detection compound. Examples of such labels include fluorescein isothiocyanate, rhodamine, Texas Red, radioisotopes (e.g., ³H, ³⁵S, ³²P, ³³P, ¹²⁵I, ¹⁴C) and colorimetric labels (such as colloidal gold, colored glass or plastic (e.g., polystyrene, polypropylene, latex, etc.) beads).

A capture polymer, as used in methods of the invention, may be attached directly or indirectly to a solid or semi-solid support. Solid materials include, for example, glass and plastic. Semi-solid materials include, for example, gelatin compounds and nitrocellulose membrane. When a capture polymer is attached directly to a solid or semi-solid support, the attachment is preferably, but not necessarily, by covalent bonds. Methods of attaching a polymer, such as a polynucleotide or oligonucleotide, to a solid or semi-solid material are well known in the art. For example, quinone photochemistry, which is available commercially as DNA Immobilizer™ from EXIQON (Vedbaek, Denmark), can be used. Quinone photochemistry is particularly useful for covalently attaching a DNA-based capture polymer to a solid polymeric material such as plastic. In another example, biotinylated capture polymers can be attached to streptavidin-coated plastic or glass surface. Capture polymers may also be indirectly attached to a support through hybridization to an extender oligonucleotide that is directly attached to the support. Indirect attachment of capture polymers is a well-known technique in the art, as described in, for example, U.S. Pat. No. 5,635,352.

Methods of the invention can be used for multiplex analysis of analytes, wherein two or more analytes comprising different sequences are detected or quantitated in a single reaction mixture. In these embodiments, a plurality of species of analyte-binding oligonucleotides and linear labeled oligonucleotides are used. A plurality of species of analyte-binding oligonucleotides would comprise two or more species of analyte-binding oligonucleotides, each species comprising (a) a sequence that is specifically hybridizable to a specific analyte and (b) a sequence that is hybridizable, directly or indirectly, to a species of linear labeled oligonucleotide. A plurality of species of linear labeled oligonucleotides would comprise two or more species of linear labeled oligonucleotides, each of which comprises a distinct label (relative to other species of the linear labeled oligonucleotide of the plurality). Each species of linear labeled oligonucleotide in the plurality further comprises a sequence that is hybridizable, directly or indirectly, to a species of analyte-binding oligonucleotide. Thus, each species of linear labeled oligonucleotide corresponds to one species of analyte-binding oligonucleotide (and thus one specific analyte). Detection of the label associated with a particular species of linear labeled oligonucleotide would thus indicate the presence of the corresponding analyte.

A single analyte can be detected by methods of the invention utilizing a single species of capture polymers or a plurality of capture polymers in a single reaction mixture. A species of capture polymer is a capture polymer comprising a specific nucleic acid sequence that is hybridizable to an analyte. Thus, a plurality of capture polymers refers to two or more species of capture polymers, each of which comprising a different analyte-binding nucleic acid sequence. In some embodiments, each species of a plurality of capture polymer species comprises a different analyte-binding nucleic acid sequence, wherein each analyte-binding sequence is hybridizable to the same analyte. In these embodiments, a single analyte may be detected or quantitated using, in a single reaction mixture, preferably at least about 1, more preferably at least about 3, even more preferably at least about 5, still more preferably at least about 6 species of capture polymers. In some embodiments, a single analyte is detected or quantitated using, in a single reaction mixture, preferably from about 1 to about 10, more preferably from about 3 to about 8, even more preferably from about 5 to about 7 species of capture polymers. In other embodiments, each species of a plurality of capture polymer species comprises a different analyte-binding nucleic acid sequence, wherein each analyte-binding sequence is hybridizable to a different analyte (i.e., two or more analytes with non-identical nucleic acid sequences). These embodiments are particularly useful in, for example, multiplex detection or quantitation of analytes.

Methods of the invention are capable of detection and quantitation of analytes present in a sample in a wide range of concentrations. In some embodiments, the concentration of analyte detectable and quantifiable by methods of the invention is preferably at least about 0.01 pg/mL, preferably at least about 70 pg/mL, preferably at least about 200 pg/mL, preferably at least about 2000 pg/mL, preferably at least about 5000 pg/mL, preferably at least about 20000 pg/mL, and preferably at least about 50000 pg/mL. In other embodiments, the concentration of analyte detectable and quantifiable by methods of the invention is preferably equal to or less than about 50000 pg/mL, preferably equal to or less than about 20000 pg/mL, preferably equal to or less than about 5000 pg/mL, preferably equal to or less than about 2000 pg/mL, preferably equal to or less than about 200 pg/mL, preferably equal to or less than about 70 pg/mL, and preferably equal to or less than about 0.01 pg/mL. In still other embodiments, the concentration of analyte detectable and quantifiable by methods of the invention is preferably from about 0.01 to about 100000 pg/mL, preferably from about 50 to about 75000 pg/mL, preferably from about 200 to about 50000 pg/mL, preferably from about 1000 to about 35000 pg/mL, and preferably from about 2000 to about 20000 pg/mL.

Methods of the invention provide high specificity of detection of nucleic acid analytes. In some embodiments, an analyte is detected with preferably less than about 5%, preferably less than about 2%, preferably less than about 1%, preferably less than about 0.5%, and preferably less than about 0.1% interference from a non-analyte nucleic acid molecule with high nucleotide sequence identity to the analyte, when the non-analyte nucleic acid molecule is present in a reaction mixture. In some embodiments, the non-analyte nucleic acid molecule with high nucleotide sequence identity preferably has equal to or less than 85%, preferably equal to or less than 80%, preferably equal to or less than 70%, preferably equal to or less than 60% sequence identity with the analyte. In certain embodiments, the non-analyte nucleic acid molecule with high nucleotide sequence identity preferably has equal to or more than 60%, preferably equal to or more than 70%, preferably equal to or more than 80%, preferably equal to or more than 82%, preferably equal to or more than 90% sequence identity with the analyte. In other embodiments, the non-analyte nucleic acid molecule with high nucleotide sequence homology preferably has from about 50% to about 90%, preferably has from about 60% to about 85%, preferably from about 70% to about 85% sequence identity with the analyte.

Methods of Detection and Quantitation Using an Analyte-Binding Linear Labeled Oligonucleotide

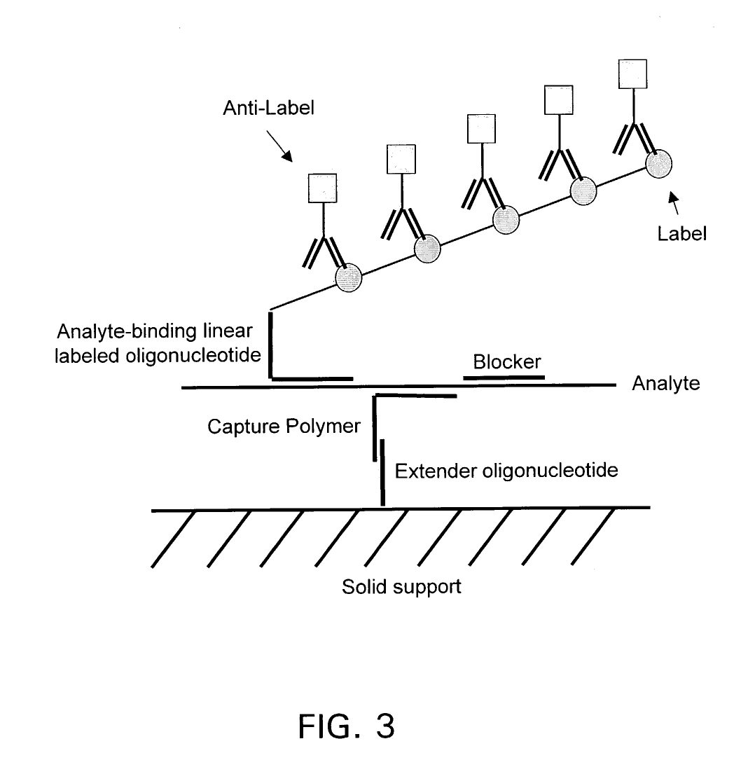

In yet another aspect, the invention provides methods of detection and quantitation wherein an analyte-binding linear labeled oligonucleotide is used to provide a means of detecting formation of a complex of analyte, capture polymer and analyte-binding linear labeled oligonucleotide. In one embodiment, one example of which is illustrated in FIG. 3, the analyte-binding linear labeled oligonucleotide of the invention comprises (a) two or more units of label each attached directly to the oligonucleotide and (b) a sequence that is hybridizable to the analyte. The capture polymer comprises a sequence that is directly or indirectly hybridizable to the analyte. A sample suspected of containing a nucleic acid analyte is contacted with an analyte-binding linear labeled oligonucleotide and a capture polymer under conditions whereby, if the analyte is present in the sample, a complex comprising the analyte, the analyte-binding linear labeled oligonucleotide and the capture polymer is formed. In general, the capture polymer is directly or indirectly attached to a support, which is generally comprised of a solid or semi-solid material. Attachment of the capture polymer to the support may be prior to, during or following the reaction wherein the complex of interest is formed. A complex of interest that is formed would remain on the surface of the support when unbound sample and/or components (i.e., analyte-binding linear labeled oligonucleotides and capture polymers) are washed away.

The complex that remains on the support can be detected in any of a number of ways. Preferably, the complex is contacted with a label-detection compound that binds to the labels on the linear labeled oligonucleotide, wherein the label-detection compound is capable of directly or indirectly generating a detectable signal. In one example, a label may be a member of a specific binding pair, such as a receptor-ligand pair or antibody-antigen pair. For example, if the label is an antigen (such as digoxigenin), an antibody specific for the antigen can be used. The antibody can itself generate a detectable signal, for example, through a signal producing moiety attached to the antibody. The antibody can also generate a detectable signal indirectly, for example, through an enzyme attached to it, which enzyme is capable of catalyzing a reaction when contacted with a substrate to produce a detectable signal. Suitable enzymes include, but are not limited to, lacZ, horseradish peroxidase, alkaline phosphatase. Other specific binding pairs are known in the art, for example ligands that have natural anti-ligands, such as biotin, thyroxine and cortisol. Various signal producing moieties and combinations are well known in the art, some of which are described herein. In instances wherein signal amplification is not desired, the labels on the linear labeled oligonucleotide can be moieties that are capable of generating a detectable signal without being first contacted with a label-detection compound. Examples of such labels include fluorescein isothiocyanate, rhodamine, Texas Red, radioisotopes (e.g., ³H, ³⁵S, ³²P, ³³P, ¹²⁵I, ¹⁴C) and colorimetric labels (such as colloidal gold, colored glass or plastic (e.g., polystyrene, polypropylene, latex, etc.) beads).

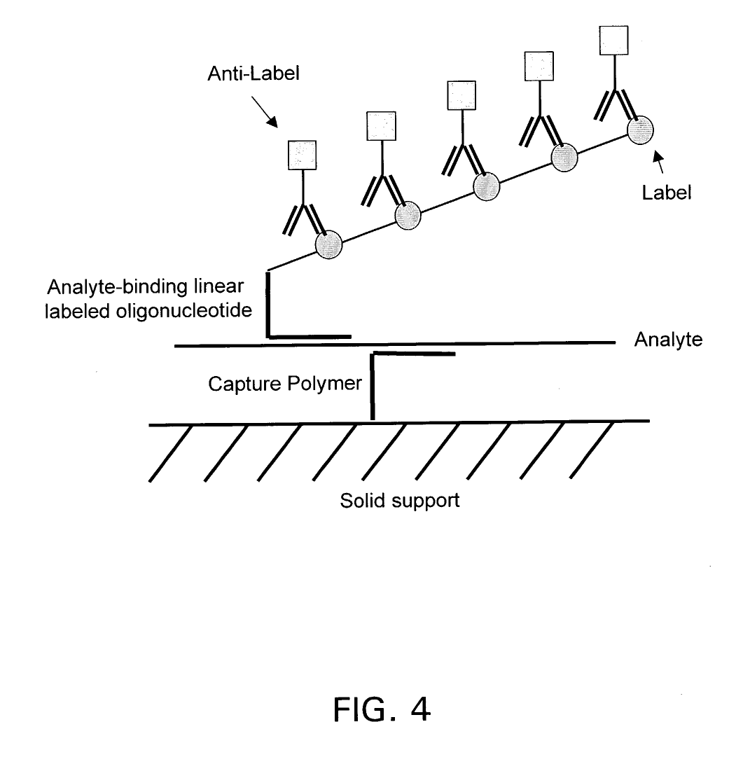

A capture polymer, as used in methods of the invention, may be attached directly (see, for example, FIG. 4) or indirectly to a solid or semi-solid support. Solid materials include, for example, glass and plastic. Semi-solid materials include, for example, gelatin compounds and nitrocellulose membrane. When a capture polymer is attached directly to a solid or semi-solid support, the attachment is preferably, but not necessarily, by covalent bonds. Methods of attaching a polymer, such as a polynucleotide or oligonucleotide, to a solid or semi-solid material are well known in the art. For example, quinone photochemistry, which is available commercially as DNA Immobilizer™ from EXIQON (Vedbaek, Denmark), can be used. Quinone photochemistry is particularly useful for covalently attaching a DNA-based capture polymer to a solid polymeric material such as plastic. In another example, biotinylated capture polymers can be attached to streptavidin-coated plastic or glass surface. Capture polymers may also be indirectly attached to a support through hybridization to an extender oligonucleotide that is directly attached to the support. Indirect attachment of capture polymers is a well-known technique in the art, as described in, for example, U.S. Pat. No. 5,635,352.

Methods of the invention can be used for multiplex analysis of analytes, wherein two or more analytes comprising different sequences are detected or quantitated in a single reaction mixture. In these embodiments, a plurality of species of analyte-binding oligonucleotides linear labeled oligonucleotides are used. A plurality of species of analyte-binding linear labeled oligonucleotides would comprise two or more species of analyte-binding linear labeled oligonucleotides, each species comprising (a) a sequence that is specifically hybridizable to a specific analyte and (b) a distinct label (relative to other species of the analyte-binding linear labeled oligonucleotides of the plurality). Thus, each species of analyte-binding linear labeled oligonucleotide corresponds to one specific analyte. Detection of the label associated with a particular species of analyte-binding linear labeled oligonucleotide would thus indicate the presence of the corresponding analyte.

A single analyte can be detected by methods of the invention utilizing a single species of capture polymers or a plurality of capture polymers in a single reaction mixture. A species of capture polymer is a capture polymer comprising a specific nucleic acid sequence that is hybridizable to an analyte. Thus, a plurality of capture polymers refers to two or more species of capture polymers, each of which comprising a different analyte-binding nucleic acid sequence. In some embodiments, each species of a plurality of capture polymer species comprises a different analyte-binding nucleic acid sequence, wherein each analyte-binding sequence is hybridizable to the same analyte. In these embodiments, a single analyte may be detected or quantitated using, in a single reaction mixture, preferably at least about 1, more preferably at least about 3, even more preferably at least about 5, still more preferably at least about 6 species of capture polymers. In some embodiments, a single analyte is detected or quantitated using, in a single reaction mixture, preferably from about 1 to about 10, more preferably from about 3 to about 8, even more preferably from about 5 to about 7 species of capture polymers. In other embodiments, each species of a plurality of capture polymer species comprises a different analyte-binding nucleic acid sequence, wherein each analyte-binding sequence is hybridizable to a different analyte (i.e., two or more analytes with non-identical nucleic acid sequences). These embodiments are particularly useful in, for example, multiplex detection or quantitation of analytes.

Methods of the invention are capable of detection and quantitation of analytes present in a sample in a wide range of concentrations. In some embodiments, the concentration of analyte detectable and quantifiable by methods of the invention is preferably at least about 0.01 pg/mL, preferably at least about 70 pg/mL, preferably at least about 200 pg/mL, preferably at least about 2000 pg/mL, preferably at least about 5000 pg/mL, preferably at least about 20000 pg/mL, and preferably at least about 50000 pg/mL. In other embodiments, the concentration of analyte detectable and quantifiable by methods of the invention is preferably equal to or less than about 50000 pg/mL, preferably equal to or less than about 20000 pg/mL, preferably equal to or less than about 5000 pg/mL, preferably equal to or less than about 2000 pg/mL, preferably equal to or less than about 200 pg/mL, preferably equal to or less than about 70 pg/mL, and preferably equal to or less than about 0.01 pg/mL. In still other embodiments, the concentration of analyte detectable and quantifiable by methods of the invention is preferably from about 0.01 to about 100000 pg/mL, preferably from about 50 to about 75000 pg/mL, preferably from about 200 to about 50000 pg/mL, preferably from about 1000 to about 35000 pg/mL, and preferably from about 2000 to about 20000 pg/mL.

Methods of the invention provide high specificity of detection of nucleic acid analytes. In some embodiments, an analyte is detected with preferably less than about 5%, preferably less than about 2%, preferably less than about 1%, preferably less than about 0.5%, and preferably less than about 0.1% interference from a non-analyte nucleic acid molecule with high nucleotide sequence identity to the analyte, when the non-analyte nucleic acid molecule is present in a reaction mixture. In some embodiments, the non-analyte nucleic acid molecule with high nucleotide sequence identity preferably has equal to or less than 85%, preferably equal to or less than 80%, preferably equal to or less than 70%, preferably equal to or less than 60% sequence identity with the analyte. In certain embodiments, the non-analyte nucleic acid molecule with high nucleotide sequence identity preferably has equal to or more than 60%, preferably equal to or more than 70%, preferably equal to or more than 80%, preferably equal to or more than 82%, preferably equal to or more than 90% sequence identity with the analyte. In other embodiments, the non-analyte nucleic acid molecule with high nucleotide sequence homology preferably has from about 50% to about 90%, preferably has from about 60% to about 85%, preferably from about 70% to about 85% sequence identity with the analyte.

Methods of Detection and Quantitation Using Capture Polymers of the Invention

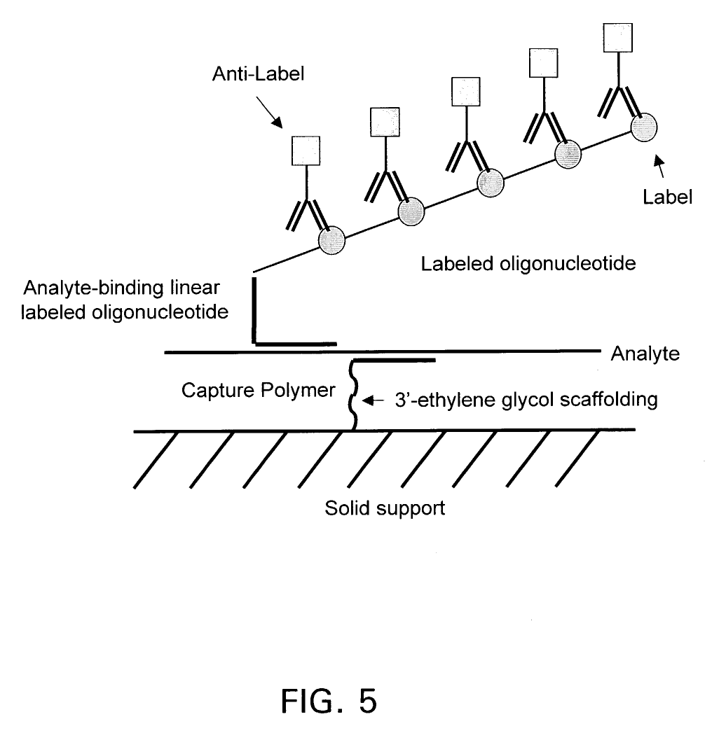

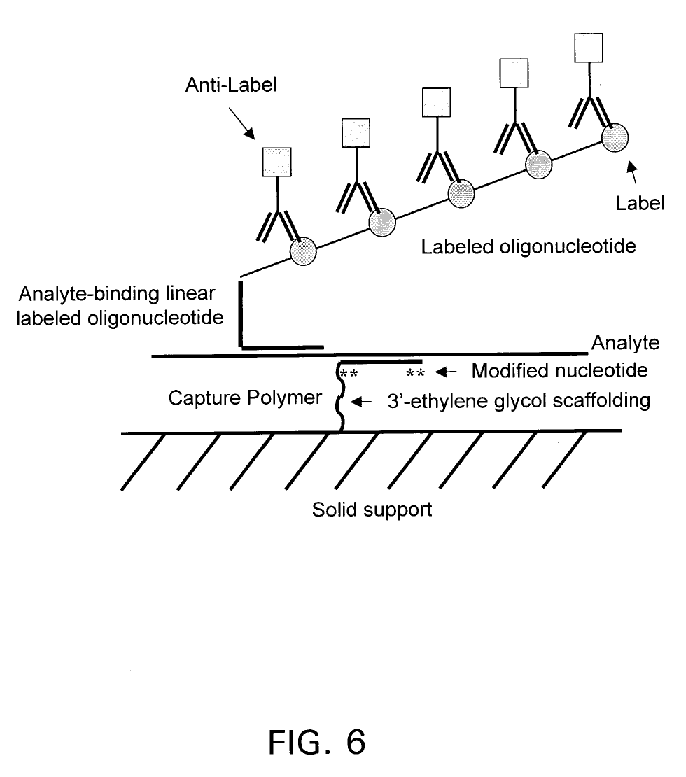

In one aspect, the invention provides methods of detection and quantitation wherein the capture polymer used to capture a nucleic acid analyte is modified to decrease non-specific binding, in particular non-specific analyte binding, to the capture polymer without substantially reducing specific binding of analyte to the capture polymer. In one aspect, one example of which is illustrated in FIG. 5, the capture polymer comprises a first portion that is hybridizable to the analyte and a second portion comprising a material (preferably but not necessarily a non-nucleic acid material) that is not substantially hybridizable to nucleic acid. In another aspect, the capture polymer comprises a sequence that is hybridizable to the analyte and further comprises at least one modified nucleotide that enhances strength of hybridization of the polymer to the analyte. In yet another aspect, an example of which is illustrated in FIG. 6, the capture polymer comprises a first portion that is hybridizable to the analyte, said first portion comprising at least one modified nucleotide that enhances strength of hybridization of the polymer to the analyte, and a second portion comprising a material (preferably but not necessarily a non-nucleic acid material) that is not substantially hybridizable to nucleic acid. Capture polymers for use in these methods are described in greater detail below.

A sample suspected of containing a nucleic acid analyte is contacted with a capture polymer and an analyte-binding oligonucleotide under conditions whereby, if the analyte is present in the sample, a complex comprising the analyte, the capture polymer and the analyte-binding oligonucleotide is formed.

In general, the capture polymer is directly or indirectly attaced to a support, which is generally comprised of a solid or semi-solid material. Attachment of the capture polymer to the support may be prior to, during or following the reaction wherein the complex of interest is formed. A complex of interest that is formed would remain on the surface of the support when unbound sample and/or components (which may include capture polymers and analyte-binding oligonucleotides) are washed away.

The complex that remains on the support can be detected in any of a number of ways. Generally, any technique that provides an indication that a complex comprising an analyte-binding oligonucleotide and an analyte is bound to the support (through complexing with the capture polymer of the invention) can be used. These techniques include those described herein. For example, in one embodiment, the analyte-binding oligonucleotide comprises both (a) a sequence hybridizable to the analyte and (b) two or more units of signaling label each attached directly to the oligonucleotide. In another embodiment, the combination of linear labeled oligonucleotides and stem oligonucleotides (which is preferably linear) of the invention as described above is used to detect a complex comprising the analyte-binding oligonucleotide and analyte. In yet another embodiment, the linear labeled oligonucleotides of the invention which comprise a sequence hybridizable to an analyte-binding oligonucleotide as described above are used to detect a complex comprising the analyte-binding oligonucleotide and analyte. Other methods of detecting a complex comprising an analyte-binding oligonucleotide and analyte are known in the art, for example as described in U.S. Pat. Nos. 5,849,481; 5,629,153; 5,624,802; 5,672,475; 5,710,264 and 5,124,246.

A capture polymer, as used in methods of the invention, may be attached directly (see, for example, FIGS. 4-6) or indirectly to a solid or semi-solid support. Solid materials include, for example, glass and plastic. Semi-solid materials include, for example, gelatin compounds and nitrocellulose membrane. When a capture polymer is attached directly to a solid or semi-solid support, the attachment is preferably, but not necessarily, by covalent bonds. Methods of attaching a polymer, such as a polynucleotide or oligonucleotide, to a solid or semi-solid material are well known in the art. For example, quinone photochemistry, which is available commercially as DNA Immobilizer™ from EXIQON (Vedbaek, Denmark), can be used. Quinone photochemistry is particularly useful for covalently attaching a DNA-based capture polymer to a solid polymeric material such as plastic. In another example, biotinylated capture polymers can be attached to streptavidin-coated plastic or glass surface. Capture polymers may also be indirectly attached to a support through hybridization to an extender oligonucleotide that is directly attached to the support. Indirect attachment of capture polymers is a well-known technique in the art, as described in, for example, U.S. Pat. No. 5,635,352.

A single analyte can be detected by methods of the invention utilizing a single species of capture polymers or a plurality of capture polymers in a single reaction mixture. A species of capture polymer is a capture polymer comprising a nucleic acid sequence that is hybridizable to a specific (a particular) analyte. Thus, a plurality of capture polymers refers to two or more species of capture polymers, each of which comprising a different analyte-binding nucleic acid sequence. In some embodiments, each species of a plurality of capture polymer species comprises a different analyte-binding nucleic acid sequence, wherein each analyte-binding sequence is hybridizable to the same analyte. In these embodiments, a single analyte may be detected or quantitated using, in a single reaction mixture, preferably at least about 1, more preferably at least about 3, even more preferably at least about 5, still more preferably at least about 6 species of capture polymers. In some embodiments, a single analyte is detected or quantitated using, in a single reaction mixture, preferably from about 1 to about 10, more preferably from about 3 to about 8, even more preferably from about 5 to about 7 species of capture polymers. In other embodiments, each species of a plurality of capture polymer species comprises a different analyte-binding nucleic acid sequence, wherein each analyte-binding sequence is hybridizable to a different analyte (i.e., two or more analytes with non-identical nucleic acid sequences). These embodiments are particularly useful in, for example, multiplex detection or quantitation of analytes.

Methods of the invention are capable of detection and quantitation of analytes present in a sample in a wide range of concentrations. In some embodiments, the concentration of analyte detectable and quantifiable by methods of the invention is preferably at least about 0.01 pg/mL, preferably at least about 70 pg/mL, preferably at least about 200 pg/mL, preferably at least about 2000 pg/mL, preferably at least about 5000 pg/mL, preferably at least about 20000 pg/mL, and preferably at least about 50000 pg/mL. In other embodiments, the concentration of analyte detectable and quantifiable by methods of the invention is preferably equal to or less than about 50000 pg/mL, preferably equal to or less than about 20000 pg/mL, preferably equal to or less than about 5000 pg/mL, preferably equal to or less than about 2000 pg/mL, preferably equal to or less than about 200 pg/mL, preferably equal to or less than about 70 pg/mL, and preferably equal to or less than about 0.01 pg/mL. In still other embodiments, the concentration of analyte detectable and quantifiable by methods of the invention is preferably from about 0.01 to about 100000 pg/mL, preferably from about 50 to about 75000 pg/mL, preferably from about 200 to about 50000 pg/mL, preferably from about 1000 to about 35000 pg/mL, and preferably from about 2000 to about 20000 pg/mL.

Methods of the invention provide high specificity of detection of nucleic acid analytes. In some embodiments, an analyte is detected with preferably less than about 5%, preferably less than about 2%, preferably less than about 1%, preferably less than about 0.5%, and preferably less than about 0.1% interference from a non-analyte nucleic acid molecule with high nucleotide sequence identity to the analyte, when the non-analyte nucleic acid molecule is present in a reaction mixture. In some embodiments, the non-analyte nucleic acid molecule with high nucleotide sequence identity preferably has equal to or less than 85%, preferably equal to or less than 80%, preferably equal to or less than 70%, preferably equal to or less than 60% sequence identity with the analyte. In certain embodiments, the non-analyte nucleic acid molecule with high nucleotide sequence identity preferably has equal to or more than 60%, preferably equal to or more than 70%, preferably equal to or more than 80%, preferably equal to or more than 82%, preferably equal to or more than 90% sequence identity with the analyte. In other embodiments, the non-analyte nucleic acid molecule with high nucleotide sequence homology preferably has from about 50% to about 90%, preferably has from about 60% to about 85%, preferably from about 70% to about 85% sequence identity with the analyte.

The number of species of capture polymer in a particular reaction can affect detection sensitivity and/or specificity. Without being bound by theory, it is noted that cooperation, flexibility and stability of hybridization of capture polymers may influence sensitivity and/or specificity of nucleic acid analyte detection. Involvement of a plurality of species of capture polymers (each species comprising a sequence that hybridizes to a different region on a particular analyte) could increase the probability for a multi-capture event, and therefore promote stronger capture. Cooperation in the capture process may also be important in determining detection specificity since only the specific target sequence is captured through the optimum number of capture sequences. Conventional nucleic acid array and microarray capture systems generally include only a single species of capture sequence per spot. The use of “universal” oligonucleotides (required in methods of the art) precludes attachment of a plurality of species of capture oligonucleotides in arrays and microarrays. In contrast, methods of the present invention, which permit direct attachment of capture polymers to a support, allow for a plurality of species of capture polymers to be provided in each array or microarray spot, thus making methods of the invention particularly suitable for adaptation to the array and microarray format. Such arrays and microarrays would provide for significant improvement of sensitivity and specificity of analyte detection and quantitation in solution phase hybridization assays, such as assays based on or designed according to methods of the invention.

In some instances, direct attachment of a capture polymer to a support may negatively affect hybridization-based assay performance, due to, for example, loss of flexibility and/or cooperativity of directly-attached capture polymers. The present invention provides methods of modifying capture polymers to compensate for any loss of assay performance that may be due to direct, as opposed to indirect, attachment of capture polymers to a support.

Components and Reaction Conditions Used in Methods of the Invention Analytes

Nucleic acid analytes referred to herein can be from a variety of sources, e.g., biological fluids or solids, food stuffs, environmental materials. Biological fluids include blood, serum, sputum, urine, semen, cerebrospinal fluid, bronchial aspirate, organ tissue, cell lysate and cell culture medium. Analytes may be prepared for the hybridization analysis by a variey of means, for example, proteinase K/SDS, chaotropic salts, etc. In some instances, the average size of the analytes may be decreased by enzymatic, physical or chemical means, e.g., using restriction enzymes, sonication, chemical degradation (e.g., metal ions), etc. Fragments may be as small as 0.1 kb, usually at least about 0.5 kb and may be 1 kb or greater. Analytes should be at least partially single stranded, preferably completely single stranded. If it is not naturally single stranded, it should first be denatured. Denaturation can be carried out by various methods known in the art, such as treatment with alkali, formamide, salts, heat, enzymes or combinations thereof.

Nucleic acid analytes can be in any form of nucleic acid capable of forming nucleic acid duplexes through base pair hydrogen bonding. Thus, nucleic acid analytes may be RNA, DNA, RNA-DNA hybrids, modified RNA and/or DNA (as known in the art and described herein) and nucleic acid complexed with proteinaceous material.

Methods of the invention can be utilized to detect and/or quantitate nucleic acid analytes comprising sequences encoding any part or all of any polypeptide, including growth hormone, insulin-like growth factors, human growth hormone, N-methionyl human growth hormone, bovine growth hormone, parathyroid hormone, thyroxine, insulin, proinsulin, relaxin, prorelaxin, glycoprotein hormones, follicle stimulating hormone (FSH), thyroid stimulating hormone (TSH), leutinizing hormone (LH), hematopoietic growth factor, vesicular endothelial growth factor (VEGF), hepatic growth factor, fibroblast growth factor, prolactin, placental lactogen, tumor necrosis factor-alpha, tumor necrosis factor-beta, mullerian-inhibiting substance, mouse gonadotropin-associated peptide, inhibin, activin, vascular endothelial growth factor, integrin, nerve growth factors (NGFs), NGF-beta, platelet-growth factor, transforming growth factors (TGFs), TGF-alpha, TGF-beta, insulin-like growth factor-I, insulin-like growth factor-II, erythropoietin (EPO), osteoinductive factors, interferons, interferon-alpha, interferon-beta, interferon-gamma, colony stimulating factors (CSFs), macrophage-CSF (M-CSF), granulocyte-macrophage-CSF (GM-CSF), granulocyte-CSF (G-CSF), thrombopoietin (TPO), interleukins (ILs), IL-1, IL-1alpha, IL-2, IL-3, IL-4, IL-5, IL-6, IL-7, IL-8, IL-9, IL-11, IL-12, LIF, SCF, neurturin (NTN) and kit-ligand (KL), HER2, human Fc, human heavy and light chains (constant region), KDR, nitric oxide synthase (NOS), angiotensin converting enzyme (ACE).

Stem Oligonucleotide

Stem oligonucleotides are useful as linker oligonucleotides that link an analyte-binding oligonucleotide and a signaling oligonucleotide that is capable of directly or indirectly generating a detectable signal. Stem oligonucleotides of the invention are preferably linear, i.e., they are not branched. Linear stem oligonucleotides are easy to develop and use due to its simplicity of structure, yet provides good specificity and sensitivity of analyte detection and quantitation, for example when used in methods of the invention. Thus, for example, when used in combination with a linear labeled oligonucleotide of the present invention (as described herein), a stem oligonucleotide comprises (a) a sequence that is hybridizable to the analyte-binding oligonucleotide used in a particular reaction mixture; and (b) a sequence that is hybridizable, directly or indirectly, to the linear labeled oligonucleotide. Generally, these two sequences are selected such that they are substantially complementary, preferably completely complementary, to the sequences to which they are intended to be hybridizable, yet not substantially hybridizable to other sequences that are present in a particular reaction mixture. For example, the sequence of the stem oligonucleotide that is hybridizable to analyte-binding oligonucleotide would be substantially complementary, preferably completely complementary, to a sequence in the analyte-binding oligonucleotide. When a sequence of the stem oligonucleotide is “indirectly” hybridizable to a labeled oligonucleotide, it is intended that the sequence is substantially complementary, preferably completely complementary, to a sequence in an intermediate (i.e., bridging) oligonucleotide which itself may be directly or indirectly hybridizable to the labeled oligonucleotide. When a sequence of the stem oligonucleotide is “directly” hybridizable to a labeled oligonucleotide, it is intended that the sequence is substantially complementary, preferably completely complementary, to a sequence in the labeled oligonucleotide. Techniques for selection of sequences that are or are not substantially hybridizable to each other are well known in the art. Whether two sequences are substantially hybridizable can also be determined empirically, and one of skill in the art recognizes that nucleic acid hybridization depends on a variety of factors, including sequence complementarity, and reaction conditions, which include ionic strength, temperature, presence of interfering substances, etc.

In some embodiments, a linear stem oligonucleotide of the invention comprises one iteration of a sequence that is hybridizable to an analyte-binding oligonucleotide. In other embodiments, a linear stem oligonucleotide of the invention comprises two or more iterations of a sequence that is hybridizable to an analyte-binding oligonucleotide. In some embodiments, each linear stem oligonucleotide of the invention comprises a single sequence (either one or more than one iteration of the sequence) that is hybridizable to an analyte-binding oligonucleotide. In other embodiments, each linear stem oligonucleotide of the invention comprises two or more distinct sequences (with one or more than one iteration of each distinct sequence) that is hybridizable to an analyte-binding oligonucleotide.

Sequences that are hybridizable between a stem oligonucleotide and an analyte-binding or signaling oligonucleotide (or an intermediate/bridging oligonucleotide as described above), respectively, are preferably of at least about 60%, preferably at least about 75%, preferably at least about 90%, preferably at least about 95%, and preferably 100% (complete) complementarity. A sequence that is hybridizable between a stem oligonucleotide and an analyte-binding or signaling oligonucleotide (or an intermediate/bridging oligonucleotide as described above) is preferably at least about 5 nucleotides, preferably at least about 10 nucleotides, preferably at least about 15 nucleotides, preferably at least about 20 nucleotides, preferably at least about 25 nucleotides in length.

A linear stem oligonucleotide of the invention is preferably at least about 5 nucleotides, preferably at least about 15 nucleotides, preferably at least about 25 nucleotides, preferably at least about 30 nucleotides, preferably at least about 45 nucleotides in length.

Linear Labeled Oligonucleotide

The linear labeled oligonucleotide of the invention is one example of an oligonucleotide that may be used to detect formation of a complex comprising an analyte-binding oligonucleotide and analyte in methods of the invention. These linear labeled oligonucleotides are simple and easy to develop and use, and thus provide a convenient form of detection oligonucleotide. The oligonucleotides comprise at least one unit of label attached directly to the oligonucleotide. Preferably, the oligonucleotides comprise two or more units of label, with each label attached directly to the oligonucleotide. The phrase “each label is attached directly to a linear labeled oligonucleotide” means the labels of the oligonucleotide are not attached on another polynucleotide/oligonucleotide that in turn is hybridized to the linear labeled oligonucleotide of the invention. A label can be attached “directly” to a linear labeled oligonucleotide by direct attachment to a nucleotide within the oligonucleotide or through one or more intermediate molecules that are attached (preferably by covalent bond) to a nucleotide within the oligonucleotide. In one embodiment, a linear labeled oligonucleotide is used in combination with a stem oligonucleotide. In this embodiment, the linear labeled oligonucleotide also comprises a sequence that is hybridizable, directly or indirectly, to the stem oligonucleotide. In another embodiment, a linear labeled oligonucleotide is used in combination with an analyte-binding oligonucleotide, without a stem oligonucleotide, in which case the linear labeled oligonucleotide comprises a sequence that is hybridizable to the analyte-binding oligonucleotide. In yet another embodiment, a linear labeled oligonucleotide is used to directly hybridize to an analyte, in which case the linear labeled oligonucleotide comprises a sequence that is hybridizable to the analyte.

The labels of the linear labeled oligonucleotide are preferably, but not necessarily, covalently attached to the backbone of the oligonucleotide. Methods of attaching labels to nucleotides are well known in the art. For example, digoxigenin (DIG)-labeled oligonucleotides can be generated using a digoxigenin tailing kit (Roche Molecular Biochemicals, Indianapolis, Ind., USA) by enzymatic labeling of oligonucleotides at their 3′ end with terminal transferase by incorporation of a mixture of deoxinucleotide triphosphates (dNTP) and DIG-dUTP in a template-independent reaction. In another example, labeled nucleotides can be used in oligonucleotide synthesis such that the labeled nucleotides are incorporated in the oligonucleotide. FITC and biotin labeled oligonucleotides can be synthesized on an ABI DNA/RNA synthesizer using standard phospharamidite chemistry.

Each linear labeled oligonucleotide of the invention may have any number of units of label (preferably at least about two). As is understood by one skilled in the art, determination of suitable numbers of units of label is dependent on a variety of factors known in the art, including, for example, the amount of detectable signal required for detection, the type of label used, etc. In some embodiments, the number of units of label is preferably at least about 2, preferably at least about 4, preferably at least about 8, preferably at least about 12, preferably at least about 15, preferably at least about 25. In some embodiments, the number of units of label is preferably from about 2 to about 50, preferably from about 8 to about 35, preferably from about 12 to about 25, preferably from about 15 to about 20. A unit of label, as used herein, refers to the number of individual label moiety of a particular type. For example, two units of the digoxigenin label refers to two digoxigenin molecules each attached to a linear labeled oligonucleotide. The labels may be attached with uniform or non-uniform spacing intervals on an individual linear labeled oligonucleotide. The spacing intervals can be such that two tandem units of label (i.e., two units of label closest to each other along the backbone of a linear oligonucleotide) are separated by at least preferably about 1, 3 or 5 nucleotides. In some embodiments, two tandem units of label are separated by preferably from about 1 to about 12, preferably from about 3 to about 10, preferably from about 5 to about 8 nucleotides. Unlabeled nucleotides in the space between labeled nucleotides can be of any type, for example iterations of a single or multiple nucleotide types. In some embodiments, the sequence between labeled nucleotides comprises iterations of adenine, for example, preferably from about 1 to about 12 adenines, preferably from about 3 to about 10 adenines, preferably from about 5 to about 8 adenines.

Any of a variety of labels known in the art may be used, some of which are described herein. These labels include, but are not limited to, an antigen, a member of a specific binding pair, a dye (such as fluorescent dye, for example, fluorescein isothiocyanate, rhodamine, Texas Red), radioisotopes and a member of a reporter-quencher pair.

Generally, sequences of a linear labeled oligonucleotide that are hybridizable to a sequence on another oligonucleotide are selected such that they are substantially complementary, preferably completely complementary, to the sequences to which they are intended to be hybridizable, yet not substantially hybridizable to other sequences that are present in a particular reaction mixture. When a sequence of the linear labeled oligonucleotide is “indirectly” hybridizable to a stem oligonucleotide, it is intended that the sequence is substantially complementary, preferably completely complementary, to a sequence in an intermediate (i.e., bridging) oligonucleotide which itself may be directly or indirectly hybridizable to the stem oligonucleotide. When a sequence of the linear labeled oligonucleotide is “directly” hybridizable to a stem oligonucleotide, it is intended that the sequence is substantially complementary, preferably completely complementary, to a sequence in the stem oligonucleotide. Techniques for selection of sequences that are or are not substantially hybridizable to each other are well known in the art. Whether two sequences are substantially hybridizable can also be determined empirically, and one of skill in the art recognizes that nucleic acid hybridization depends on a variety of factors, including sequence complementarity, and reaction conditions, which include ionic strength, temperature, presence of interfering substances, etc.

Sequences that are hybridizable between a linear labeled oligonucleotide and an analyte-binding, stem or intermediate (bridging) oligonucleotide, respectively, are preferably of at least about 60%, preferably at least about 75%, preferably at least about 90%, preferably at least about 95%, and preferably 100% complementarity. A sequence that is hybridizable between a linear labeled oligonucleotide and another oligonucleotide is preferably at least about 5 nucleotides, preferably at least about 10 nucleotides, preferably at least about 15 nucleotides, preferably at least about 20 nucleotides, preferably at least about 25 nucleotides in length.

A linear labeled oligonucleotide of the invention is preferably at least about 5 nucleotides, preferably at least about 15 nucleotides, preferably at least about 25 nucleotides, preferably at least about 30 nucleotides, preferably at least about 45 nucleotides in length.

Analyte-Binding Oligonucleotide

An analyte-binding oligonucleotide used in methods of the invention is an oligonucleotide comprising a sequence that is hybridizable to an analyte. When the analyte-binding oligonucleotide is not labeled, it further comprises a sequence that is hybridizable, either directly or indirectly, to a labeled oligonucleotide. For example, when used in combination with a stem oligonucleotide and a labeled oligonucleotide (which can be in any form, including the linear labeled oligonucleotide of the invention) to which the stem oligonucleotide is hybridizable, the analyte-binding oligonucleotide further comprises a sequence that is hybridizable to the stem oligonucleotide. In another example, when used in combination with a labeled oligonucleotide (which can be in any form, including the linear labeled oligonucleotide of the invention), without a stem oligonucleotide, the analyte-binding oligonucleotide further comprises a sequence that is hybridizable to the labeled oligonucleotide. In some embodiments of the invention, an analyte-binding oligonucleotide is itself labeled, for example, in the form of a linear labeled oligonucleotide of the invention which also comprises a sequence that is hybridizable to an analyte. Binding of an analyte-binding oligonucleotide to an analyte is detected through detection of the label associated with the analyte-binding oligonucleotide. Methods of detecting such labels are well known in the art, some of which are described herein.

By appropriate selection of the sequence of an analyte-binding oligonucleotide that is hybridizable to an analyte, the analyte-binding oligonucleotide can be used to identify and/or quantify a specific nucleic acid analyte. The sequence of analyte-binding oligonucleotide that is hybridizable to an analyte is preferably at least about 60%, preferably at least about 75%, preferably at least about 85%, preferably at least about 90%, preferably at least about 95%, preferably 100% complentary to the analyte sequence to which it is intended to be hybridizable. In some embodiments, the percent complementarity is preferably from about 60% to about 100%, preferably from about 70% to about 95%, preferably from about 80% to about 99%, preferably from about 90% to about 98%. A sequence that is hybridizable between an analyte-binding oligonucleotide and an analyte is preferably at least about 5 nucleotides, preferably at least about 10 nucleotides, preferably at least about 15 nucleotides, preferably at least about 20 nucleotides, preferably at least about 25 nucleotides in length.

Generally, sequences of an analyte-binding oligonucleotide that are hybridizable to an analyte or a sequence on another oligonucleotide are selected such that they are substantially complementary, preferably completely complementary, to the sequences to which they are intended to be hybridizable, yet not substantially hybridizable to other sequences that are present in a particular reaction mixture. Techniques for selection of sequences that are or are not substantially hybridizable to each other are well known in the art. Whether two sequences are substantially hybridizable can also be determined empirically, and one of skill in the art recognizes that nucleic acid hybridization depends on a variety of factors, including sequence complementarity, and reaction conditions, which include ionic strength, temperature, presence of interfering substances, etc.

Sequences that are hybridizable between an analyte-binding oligonucleotide and another oligonucleotide are preferably of at least about 60%, preferably at least about 75%, preferably about 90%, preferably at least about 95%, and preferably 100% complementarity. A sequence that is hybridizable between an analyte-binding oligonucleotide and another oligonucleotide is preferably at least about 5 nucleotides, preferably at least about 10 nucleotides, preferably at least about 15 nucleotides, preferably at least about 20 nucleotides, preferably at least about 25 nucleotides in length.

An analyte-binding oligonucleotide is preferably at least about 5 nucleotides, preferably at least about 15 nucleotides, preferably at least about 25 nucleotides, preferably at least about 30 nucleotides, preferably at least about 45 nucleotides in length.

Capture Polymer

Capture polymers of the invention comprise a sequence that is hybridizable directly or indirectly to an analyte. The sequence is preferably hybridizable directly to an analyte, i.e., it has a sufficient degree of complementary to an analyte sequence such that it is capable of hybridizing to the analyte under reaction conditions. In embodiments wherein a capture polymer is hybridizable indirectly to an analyte, a linker oligonucleotide that links the capture polymer to an analyte may be used. A capture polymer as used in methods of the invention serves to immobilize an analyte when a complex of the analyte and capture polymer is formed. Detection of the complex, for example through binding of an analyte-binding oligonucleotide to the analyte in the capture polymer-analyte complex, indicates the presence and/or quantity of the analyte in a sample.

Any form of capture polymer with the characteristics described above may be used, including capture polymers of the invention. In one embodiment, the invention provides a capture polymer comprising a first portion that is hybridizable to an analyte and a second portion comprising a material (preferably but not necessarily a non-nucleic acid material) that is not substantially hybridizable to nucleic acid. In some examples of these embodiments, the capture polymer is comprised of a combination of nucleotides and a material(s) (generally, but not necessarily non-nucleic acid material) that is not substantially hybridizable to nucleic acid. Examples of suitable materials that are not substantially hybridizable to nucleic acid for use in methods of the invention are known in the art and can be determined empirically. For example, a suitable material is inert carbon, which can be provided in a number of molecular form, including, for example, ethylene glycol (for example, with the chemical structure of 18-O-Dimethoxytritylhexaethyleneglycol, 1-[(2-cyanoethyl)-(N,N-diisopropyl)]-phosphoramidite. In some embodiments, preferably at least about 10%, preferably at least about 25%, preferably at least about 40%, preferably 50% of the length of a capture polymer is a material that is not substantially hybridizable to nucleic acid. In other embodiments, from preferably about 5% to about 90%, preferably about 10% to about 70%, preferably about 20% to about 50% of the length of a capture polymer is a material that is not substantially hybridizable to nucleic acid. These percentages can be calculated as the number of molecules of the material that is not substantially hybridizable to nucleic acid in the backbone chain of the capture polymer as a percentage function of the total number of molecules within the backbone chain of the capture polymer.

Sequences that are hybridizable between a capture polymer and analyte are preferably of at least about 60%, preferably of at least about 75%, preferably of at least about 90%, preferably of at least about 95%, and preferably 100% complementarity. A sequence that is hybridizable between a capture polymer and analyte is preferably at least about 5 nucleotides, preferably at least about 10 nucleotides, preferably at least about 15 nucleotides, preferably at least about 20 nucleotides, preferably at least about 25 nucleotides in length.

In some embodiments, a capture polymer comprises preferably at least about 5 nucleotides, preferably at least about 15 nucleotides, preferably at least about 25 nucleotides, preferably at least about 30 nucleotides, preferably at least about 45 nucleotides. In some embodiments, a capture polymer comprises preferably from about 10 to about 60 nucleotides, preferably from about 15 to about 50 nucleotides, preferably from about 20 to about 40 nucleotides.

In some embodiments, the invention provides a capture polymer comprising a sequence that is hybridizable to an analyte and at least one modified nucleotide that enhances strength of hybridization of the polymer to the analyte. A modified nucleotide “enhances strength of hybridization” of a capture polymer to analyte if a complex comprising analyte and a first capture polymer (with modified nucleotide) is more stable than a complex comprising analyte and a second capture polymer (without said modified nucleotide), wherein the first and second capture polymers are otherwise identical. Complex stability can be determined by methods well known in the art. For example, two parallel reactions with identical components and conditions, except for whether the capture polymer comprises a modified nucleotide, are performed, and amount of complexes comprising analyte and the capture polymers determined (for e.g., by size and/or a unique characteristic of the complex based, for example, on presence of a unique detectable sequence in the complex) at the end of reaction.

Suitable modified nucleotides are known in the art and can be determined empirically. Examples of suitable modified nucleotides include, but are not limited to, locked nucleic acids, peptide nucleic acids and 2′-O-methoxy deoxyribonucleotide. Modified nucleotides can be located at any position within the sequence (referred to hereinafter as “binding sequence”) of the capture polymer that is hybridizable to an analyte (or a linker oligonucleotide). In some embodiments, a modified nucleotide is located within the 3′ portion of the binding sequence of the capture polymer. In some embodiments, a modified nucleotide is located within the 5′ portion of the binding sequence of the capture polymer. In other embodiments, a modified nucleotide is located towards the center of the binding sequence of the capture polymer. In some embodiments, modified nucleotides are located in any combination of these positions.

In some embodiments of capture polymers that comprise at least one modified nucleotide that enhances strength of hybridization of the polymer to an analyte, the capture polymer preferably comprises at least about 1, preferably at least about 3, preferably at least about 5, preferably at least about 7 such modified nucleotides. In some embodiments, preferably at least about 10%, preferably at least about 20%, preferably at least about 30%, preferably at least about 40%, preferably at least about 50% of the total number of nucleotides in the capture polymer are such modified nucleotides. In other embodiments, preferably from about 10 to about 50%, preferably from about 20 to about 40%, preferably from about 30 to about 35% of the total number of nucleotides in a capture polymer are such modified nucleotides.

The invention also provides capture polymers comprising a first portion that is hybridizable to an analyte, said first portion comprising at least one modified nucleotide that enhances strength of hybridization of the polymer to the analyte, and a second portion comprising a material (preferably but not necessarily a non-nucleic acid material) that is not substantially hybridizable to nucleic acid. Characteristics of the first portion and second portion can be any combination of those described above.

In some embodiments of the capture polymers described herein, a capture polymer comprises a spacer component useful for extending a capture polymer away from the surface of a support to which it is attached. In some embodiments, the spacer component is the material of the second portion which is not substantially hybridizable to nucleic acid. Thus, the spacer component can comprise, for example, inert carbon, which can be provided in a number of molecular form, including, for example, ethylene glycol (for example, with the chemical structure of 18-O-Dimethoxytritylhexaethyleneglycol, 1-[(2-cyanoethyl)-(N,N-diisopropyl)]-phosphoramidite. In some embodiments, the spacer component comprises preferably at least one, preferably at least three, preferably at least four C18 spacers (the chemical structure of which is described herein and known in the art). In some embodiments, the spacer component comprises preferably from about 1 to about 12, preferably from about 1 to about 8, preferably from about 3 to about 6 C18 spacers.

Reaction Conditions and Detection

Reaction conditions suitable for methods of the invention are well known in the art, for example those described in U.S. Pat. Nos. 5,849,481; 5,629,153; 5,624,802; 5,672,475; 5,710,264 and 5,124,246 and in the Examples below. Criteria for designing appropriate conditions specific to particular circumstances (such as the sample source/type, buffers, etc.) are well known, for example in nucleic acid sandwich assays, and can be routinely determined empirically.

In general, the ratio of the various oligonucleotide/polymer components of a reaction to anticipated moles of analyte would each be at least stoichiometric, and preferably in excess. This ratio is preferably, but not necessarily, 1.5:1, and more preferably 2:1. It would generally be in the range of 2:1 to 10⁶ or 10⁷:1. Concentrations of each oligonucleotide or capture polymer would generally range from about 10⁻⁴ to 10⁻¹⁰M, with sample analyte concentrations varying from 10⁻²² to 10⁻¹²M. Hybridization steps can take from about 2 minutes to about 24 hours, frequently being completed in about 1 hour or less. The reduced number of components and steps involved in methods of the invention, compared to methods known in the art, can provide great reduction in assay times. Hybridization can be carried out at any appropriate temperatures determined, at least in part, by the melting temperatures for the hybridization of the various nucleic acid sequences in a reaction. Exemplary temperatures include, but are not limited to, about 20° C. to about 80° C., more usually from about 35° C. to about 70° C., and particularly 53° C.

Hybridization reactions are generally performed in aqueous media, for example a buffer solution, which may include various additives. Suitable aqueous media are known in the art, including those described in the Examples below. Additives which may be used include low concentrations of detergent (for example, SDS at 0.1 to 1%), salts (for example, sodium citrate in exemplary concentrations ranging from 0.017 to 0.17M), salmon sperm DNA (at a concentration of, for example, 50 ug/ml) and blocking solution (for example, the blocking solution available commercially from Boehringer Mannheim (Indianapolis, Ind., USA) used at 10% concentration).

Stringency of hybridization medium may be controlled by varying various factors known in the art, for example temperature, salt concentration, solvent system. Stringency may be varied depending upon, for example, length and nature of the hybridizable sequences.

Conditions for detection of specific label types are well known in the art.

A sample suspected of containing an analyte can be contacted with the various oligonucleotide and capture polymer components simultaneously or in separate hybridization steps (which can be in any sequence, so long as the desired complex and detection/quantitation thereof is achieved). For example, a sample may first be contacted with a capture polymer, followed by a washing step to remove unbound sample (if the capture polymer is already attached to a support), and the capture polymer-analyte complex may be contacted with an analyte-binding oligonucleotide. If a labeled oligonucleotide is used to detect formation of the complex comprising analyte-binding oligonucleotide, analyte and capture polymer, the labeled oligonucleotide may be (but is not necessarily) added at the same time or subsequent to contact of the capture polymer-analyte complex with the analyte-binding oligonucleotide. Similarly, a stem oligonucleotide, if used, may be (but is not necessarily) added at the same time or subsequent to contact of the capture polymer-analyte complex with the analyte-binding oligonucleotide. If reactions conditions permit, all the oligonucleotide and capture polymer components used as described in methods of the invention may be contacted with a sample simultaneously. Generally, prior to detection of label, the complex comprising analyte, capture polymer and analyte-binding oligonucleotide (and stem oligonucleotide and/or labeled oligonucleotide, as appropriate) is washed to remove unbound sample, and/or to remove unhybridized oligonucleotides and/or capture polymers.

Microarrays Comprising Capture Polymers of the Invention

As described above, capture polymers of the invention may be attached directly to a solid or semi-solid support for use in methods of the invention. This makes capture polymers of the invention particularly suitable to be provided in the form of microarrays. Microarrays find use in various applications and provide great convenience as they permit automation, high throughput analyte analysis, and can be provided in ready-to-use packaged form. These microarrays are particularly suitable for use as the source of capture polymers in methods of the invention.

Capture polymers of the invention can be attached to a solid or semi-solid support or surface, which may be made, e.g., from glass, plastic (e.g., polypropylene, nylon), polyacrylamide, nitrocellulose, or other materials.

Several techniques are well-known in the art for attaching capture polymers to a solid substrate such as a glass slide. One method is to incorporate modified bases or analogs that contain a moiety that is capable of attachment to a solid substrate, such as an amine group, a derivate of an amine group or another group with a positive charge, into the capture polymer. The capture polymer is then contacted with a solid substrate, such as a glass slide, which is coated with an aldehyde or another reactive group which will form a covalent link with the reactive group that is on the capture polymer and becomes covalently attached to the glass slide. Other methods, such as those using amino propryl silican surface chemistry are also known in the art, as disclosed at, for e.g., http://www.cmt.corning.com and http://cmgm.Stanford.ecu/pbrown1. Methods based on quinone photochemistry are described herein.

Each discrete spot of a microarray may comprise a single species of capture polymers or a plurality of species of capture polymers, as described above.

Kits and Compositions

The invention also provides compositions, kits and articles of manufacture used in the methods described herein. The compositions may be any component(s), reaction mixture and/or intermediate described herein, as well as any combination. For example, the invention provides a composition comprising a capture polymer, wherein the capture polymer comprises a first portion that is hybridizable to an analyte and a second portion comprising a material (preferably a non-nucleic acid material) that is not substantially hybridizable to nucleic acid. The invention also provides compositions comprising a capture polymer that comprises a sequence that is hybridizable to an analyte and further comprises at least one modified nucleotide that enhances strength of hybridization of the capture polymer to the analyte. In any of these compositions, the modified nucleotide may have one or any combination of the characteristics described herein (for example, the type of modification, location of modified nucleotide, etc.). The invention also provides a composition comprising a capture polymer comprising a first portion that is hybridizable to an analyte, said first portion comprising at least one modified nucleotide that enhances strength of hybridization of the polymer to the analyte, and a second portion comprising a material (preferably a non-nucleic acid material) that is not substantially hybridizable to nucleic acid. In some embodiments, the capture polymer also comprises a spacer component, which in some embodiments comprises a material (preferably a non-nucleic acid material) that is not substantially hybridizable to nucleic acid. In some embodiments, the material (preferably a non-nucleic acid material) that is not substantially hybridizable to nucleic acid is inert carbon, which in some embodiments is provided as ethylene glycol.

The invention also provides compositions comprising a linear stem oligonucleotide, an analyte-binding oligonucleotide and a linear labeled oligonucleotide of the invention, individually or in any combination thereof. These oligonucleotides can have any one or combination of the characteristics described herein. In some embodiments, compositions comprise one or a combination of these oligonucleotides and a capture polymer of the invention.

The compositions are generally in a suitable medium, although they can be in lyophilized form. Suitable media include, but are not limited to, aqueous media (such as pure water or buffers).

The invention also provides reaction mixtures (or compositions comprising reaction mixtures) comprising any of the oligonucleotides and/or capture polymers of the invention, either with or without analyte. The invention also provides reaction intermediates obtained in carrying out methods of the invention. Thus, in one example, the invention provides a complex comprising analyte, capture polymer, analyte-binding oligonucleotide, linear stem oligonucleotide and labeled oligonucleotide (which is preferably a linear labeled oligonucleotide of the invention). In another example, the invention provides a complex comprising analyte, capture polymer, analyte-binding oligonucleotide and labeled oligonucleotide (which is preferably a linear labeled oligonucleotide of the invention). In still another example, the invention provides a complex comprising analyte, capture polymer and analyte-binding linear labeled oligonucleotide. In yet another example, the invention provides a complex comprising capture polymer and analyte. In another example, the invention provides a complex comprising analyte, analyte-binding oligonucleotide, linear stem oligonucleotide and labeled oligonucleotide (which is preferably a linear labeled oligonucleotide). In one example, the invention provides a complex comprising analyte, analyte-binding oligonucleotide and labeled oligonucleotide (which is preferably a linear labeled oligonucleotide). In yet another example, the invention provides a complex comprising analyte and analyte-binding linear labeled oligonucleotide.

The invention also provides kits and articles of manufacture for carrying out methods of the invention. Accordingly, a variety of kits and articles of manufacture are provided in suitable packaging. The kits and articles of manufacture may be used for any one or more of the uses and methods described herein, and, accordingly, may contain instructions for any one or more of these uses and methods.

The kits and articles of manufacture of the invention comprise one or more containers comprising any combination of the oligonucleotides and capture polymers described herein, and the following are examples of such kits and articles of manufacture. A kit or article of manufacture may comprise any of the capture polymers described herein. In some embodiments, a kit or article of manufacture comprises two or more species of capture polymers, which may or may not be separately packaged. The capture polymers may be attached to a solid or semi-solid support. In some embodiments, a kit or article of manufacture comprises a capture polymer and an analyte-binding oligonucleotide. A kit or article of manufacture may optionally comprise a linear stem oligonucleotide and/or labeled oligonucleotide (which is preferably a linear labeled oligonucleotide of the invention). A kit or article of manufacture may optionally comprise a label detection compound and/or necessary reagents for generation and/or detection of signal. Kits and articles of manufacture may also include one or more suitable buffers (as described herein). One or more reagents in a kit or article of manufacture can be provided as a dry powder, usually lyophilized, including excipients, which on dissolution will provide for a reagent solution having the appropriate concentrations for performing any of the methods described herein. Each component can be packaged in separate containers or some components can be combined in one container where cross-reactivity and shelf life permit.

Kits and articles of manufacture of the invention may optionally include a set of instructions, generally written instructions, although electronic storage media (e.g., magnetic diskette or optical disk) containing instructions are also acceptable, relating to the use of components of the methods of the invention. The instructions generally include information as to reagents (whether included or not in the kit) necessary for practicing the methods of the invention, instructions on how to use the kit, and/or appropriate reaction conditions. In some embodiments, kits may include an algorithm, such as one of those described herein, for designing the oligonucleotides and capture polymers used in methods of the invention. Such algorithms may be provided in written form, or as part of a storage device (such as a diskette and compact disk), together with or separately from kits as described herein.

The component(s) of a kit or article of manufacture may be packaged in any convenient, appropriate packaging. The components may be packaged separately, or in one or multiple combinations. The relative amounts of the various components in the kits and articles of manufacture can be varied widely to provide for concentrations of the reagents that substantially optimize the reactions that need to occur to practice methods of the invention and/or to further optimize the sensitivity of any method.

The following Examples are provided to illustrate, but not limit, the invention.

### EXAMPLES

### Example 1

Detection and Quantitation of Analyte Using a Linear Stem Oligonucleotide, Linear Labeled Oligonucleotide, Analyte-Binding Oligonucleotide and Capture Polymer Indirectly Attached to a Solid Support

Reaction buffers are as described below:

| Lysis Buffer (per 1 L) | | |
|---|---|---|
| 1M HEPES, pH 8.0 | 78 | ml |
| 10% Lithium lauryl sulfate | 100 | ml |
| 0.25 M EDTA | 32 | ml |
| 5M Lithium Chloride | 100 | ml |
| Proteinase K (Boehringer Mannheim) | 600 | mg |
| Micro-protect (Boehringer Mannheim) | 5 | ml |
| SQ water | q.s. to 1L | |
| Buffer is filtered. | | |
| Coating Buffer | | |
| 100 mM sodium carbonate, pH 9.6 | | |
| Coat wash buffer | | |
| 2X SSC/0.1% Tween-20 | | |
| Capture hybridization buffer | | |
| 6X SSC (20X) | 150 | ml |
| 0.1% SDS (20%) | 2.5 | ml |
| 50 ug/ml salmon sperm DNA (10 mg/ml) | 2.5 | ml |
| SQ water | 500 | ml |
| Stem/labeled oligonucleotide buffer | | |
| 6X SSC (20X) | 150 | ml |
| 10% Boehringer Mannheim Block | 50 | ml |
| SQ water | 300 | ml |
| Antibody diluent:Boehringer Mannheim Block | | |
| Maleic acid (Boehringer Mannheim) | 25 | ml |
| Block buffer (Boehringer Mannheim) | 25 | ml |
| SQ water | 200 | ml |
| Wash buffer 1 | | |
| 0.1X SSC/0.1% SDS | | |
| Wash buffer 2 | | |
| 0.1XSSC | | |

Anti-Label Antibodies

Alkaline phosphatase-conjugated anti-digoxigenin

Alkaline phosphatase-conjugated anti-FITC

Alkaline phosphatase-conjugated streptavidin

Reaction conditions and steps are as follows:

Coating

Dilute NH₂-oligonucleotide to 0.1 μm in coating buffer

Add 100 μl/well and incubate at room temperature for 2 hours in the dark under agitation

1st Hybridization

Dilute capture polymer and analyte-binding oligonucleotide into lysis buffer

Prepare samples in lysis buffer

Wash plate with coat wash buffer (3 times)

Load 50 μl/well of capture hybridization buffer and 50 μl/well of sample in lysis buffer

Mix gently and incubate overnight at 53° C. in the dark

2nd Hybridization

Cool plates for 10 minutes at room temperature

Prepare stem oligonucleotide in stem/labeled oligonucleotide buffer

Wash plate with wash buffer 1 (2 times)

Load 50 μl/well of stem oligonucleotide solution

Incubate at 53° C. for 30 minutes

3rd Hybridization

Cool plates for 10 minutes at room temperature

Prepare labeled oligonucleotide in stem/labeled oligonucleotide buffer

Wash plate with wash buffer 1 (2 times)

Load 50 μl/well of labeled oligonucleotide solution

Incubate at 53° C. for 30 minutes

Label Detection

Cool plates for 10 minutes at room temperature before washing with wash buffer 1 (2 times)

Prepare anti-label antibody in diluent and load 50 μl/well

Incubate at room temperature for 30 minutes under agitation and wash with wash buffer 1 (2 times) and 2 (3 times)

Add 50 μl/well of substrate solution and incubate at 37° C. for 1 hour before reading chemiluminescence

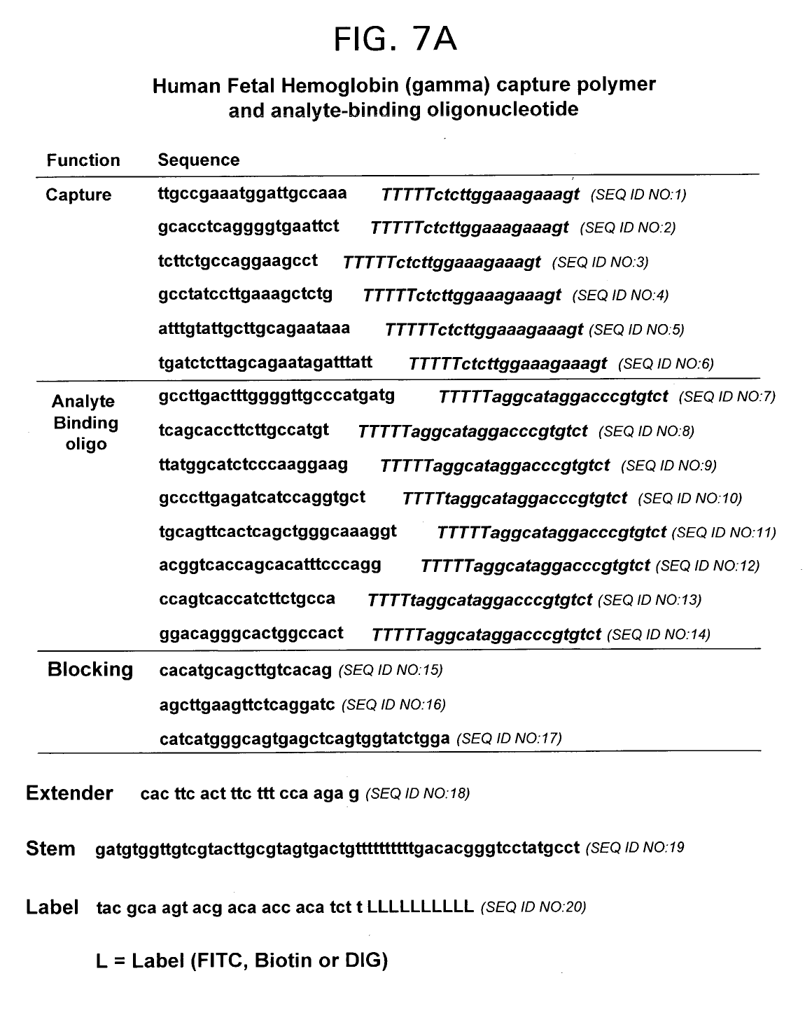

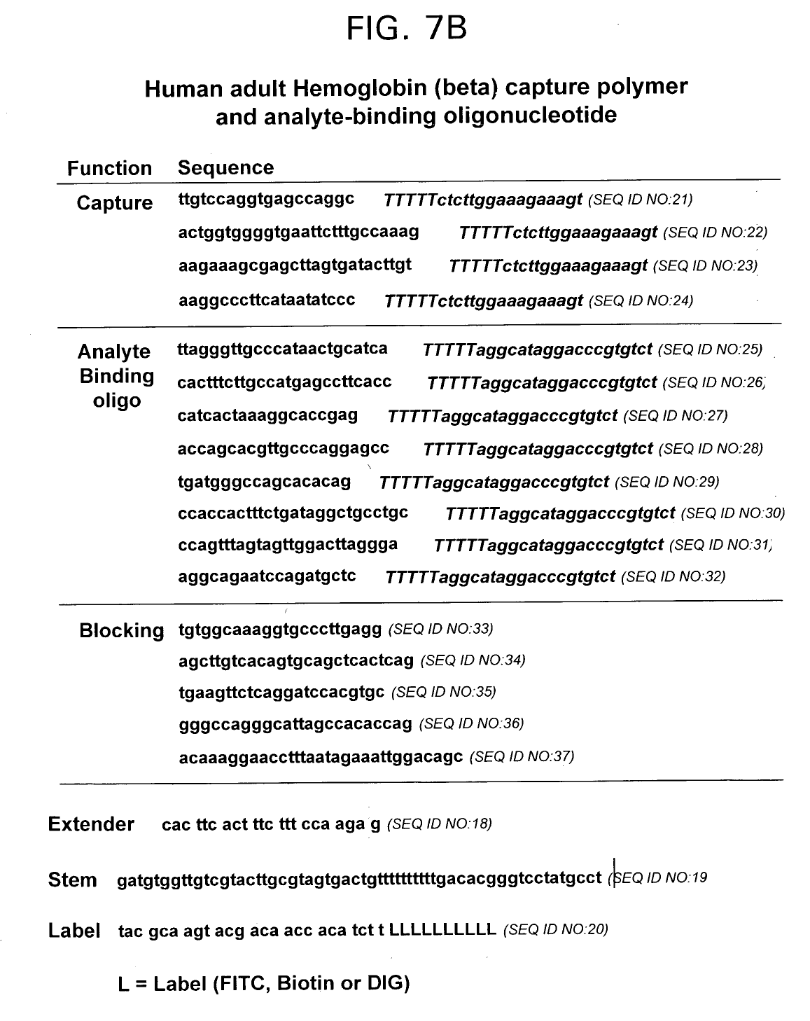

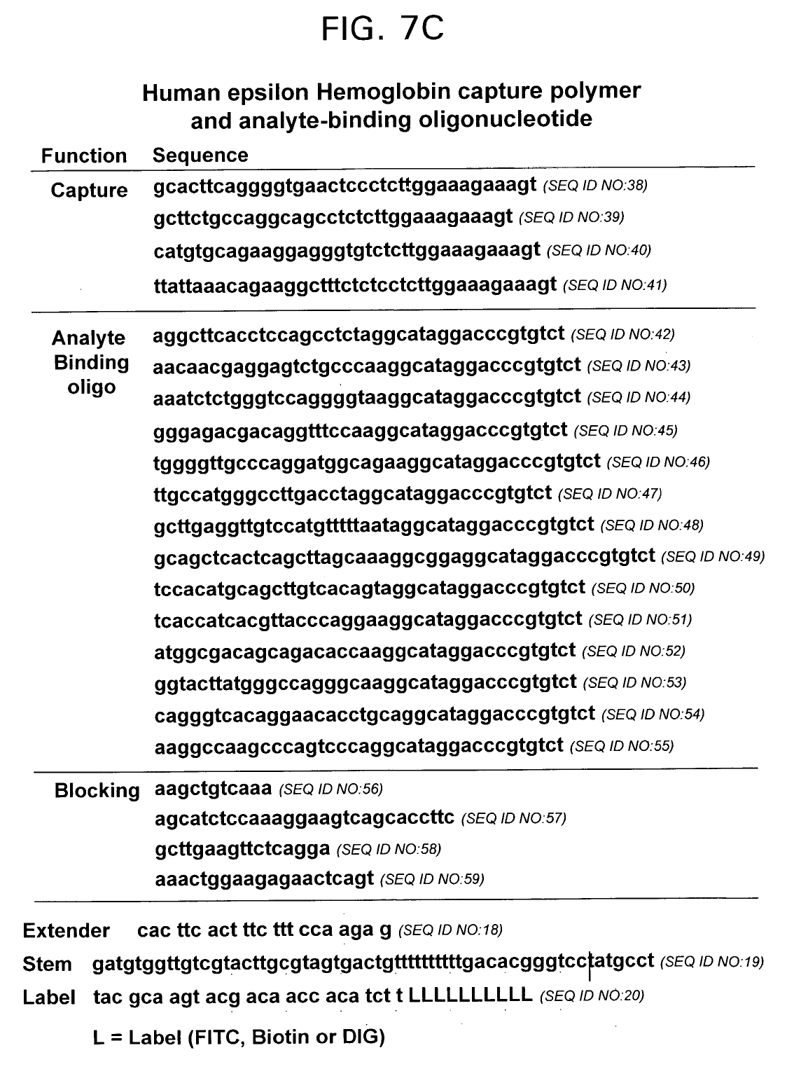

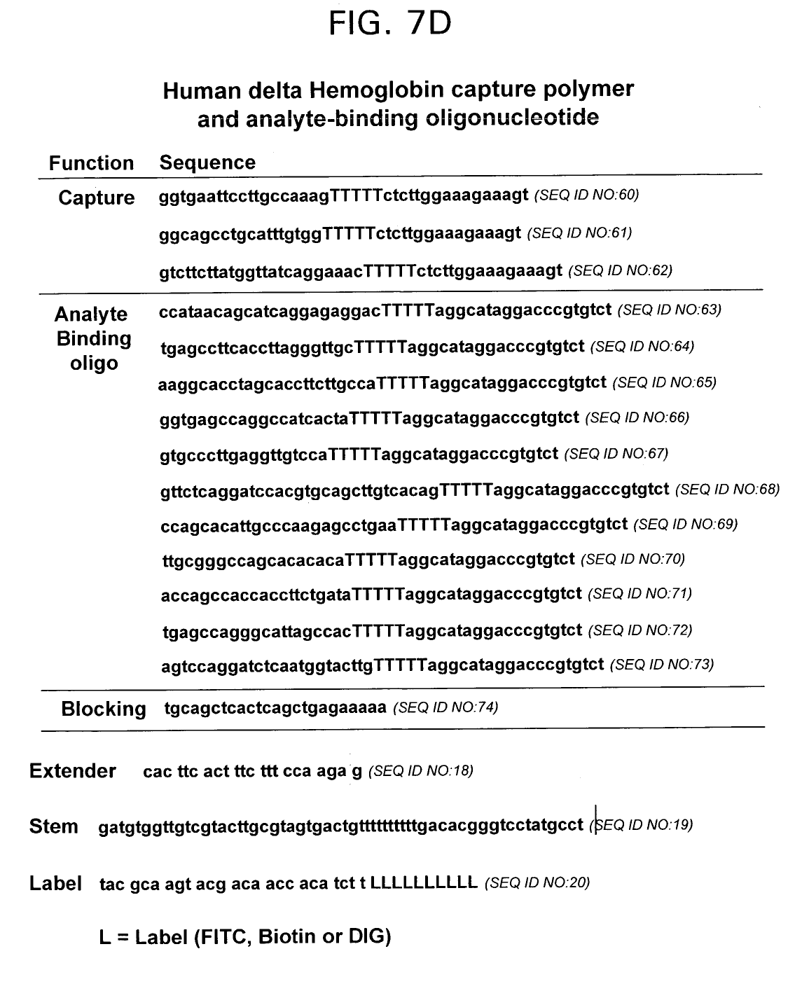

The analyte can be, for example, human fetal and adult hemoglobin provided as RNA from cell lysates, such as lysates of immortalized DB cells (hematopoietic stem cells). Sequences of the capture polymers, analyte-binding oligonucleotides and blocker oligonucleotides that may be used in detecting human fetal hemoglobin are depicted in FIG. 7A.

Sequences of the capture polymers, analyte-binding oligonucleotides and blocker oligonucleotides that may be used in detecting human beta (adult), epsilon and delta hemoglobins are depicted in FIGS. 7B, C and D, respectively.

The sequences for the various oligonucleotides (other than extender oligonucleotide) and capture polymers can be designed using the ProbeDesigner software (Chiron, Emeryville, Calif., USA). Oligonucleotides and capture polymers are synthesized using standard phosphoramidite chemistry on, for example, an ABI DNA/RNA synthesizer according to manufacturer specifications.

Linear labeled oligonucleotides are labeled with either digoxigenin, fluorescein isothiocyanate (FITC) or biotin. Digoxigenin-labeled oligonucleotides are generated using a digoxigenin oligonucleotide tailing kit (Roche Molecular Biochemicals, Cat. No. 1-417-231, Indianapolis, USA) according to manufacturer instructions. FITC and biotin-labeled oligonucleotides are generated on an ABI DNA/RNA synthesizer using standard phosphoramidite chemistry. Labels are detected using alkaline phosphatase-conjugated antibody against digoxigenin or FITC, or alkaline-phosphatase-conjugated streptavidin, as appropriate to the label on the linear labeled oligonucleotide used in a particular reaction. Detectable signal is read on an MLX microplate luminometer (Dynex, Chantilly, Va., USA).

Extender oligonucleotides, which have an NH₂ group on one end, are attached to EXIQON Immobilizer DNA plates by the coating step described above. Extender oligonucleotides comprise at their free ends a sequence that is complementary to a sequence in the capture polymers.

Data, expressed as signal to noise ratio, generated by this method of the invention can be compared to data generated using conventional methods, such as the method of Urdea et al. (as described in, for example, U.S. Pat. No. 5,635,352).

The specificity of analyte detection and quantitation by the method of the invention can be determined by analyzing the signal obtained for either the beta, epsilon or delta human hemoglobin RNA in the gamma human hemoglobin assay. There is a high homology between the hemoglobin genes (more than 82%). The amount of either beta, epsilon or delta human hemoglobin RNA detected in the gamma human hemoglobin assay, expressed as a percentage of amount of gamma human hemoglobin RNA detected in the gamma hemoglobin assay, would indicate the percentage of interference by a non-specific sequence(s), thus indicating the specificity of analyte detection and quantitation.

To determine the dynamic range of concentrations over which the method can be used to detect and quantitate analyte, detection over a range of analyte concentrations can be performed. The range over which the signal values remain linear would represent the dynamic concentration range.

To determine the effects, if any, of choice of label used in the linear labeled oligonucleotides, the signaling performance of digoxigenin-labeled and FITC-labeled linear labeled oligonucleotides can be compared. To determine the effects, if any, of spacing between tandem units of label in the linear labeled oligonucleotides, spacing between FITC and biotin in the respective oligonucleotides can be modulated during oligonucleotide synthesis. Space between labels can be filled with, for example, 3 to 8 adenine molecules.

The assay method as described above may utilize direct attachment of capture polymers to the plates, rather than indirectly through the extender oligonucleotides. Reaction components (excluding extender oligonucleotides) and conditions can be according to those described above. One end of the capture polymer would have an NH₂ group to allow its direct attachment to the DNA Immobilizer assay plate (EXIGON).

### Example 2

Detection and Quantitation Using Capture Polymer Attached Directly to Solid Support

Reaction buffers were as described in Example 1.

Effect of Direct Attachment of Capture Polymers to Solid Support

Reaction conditions and steps were as follows:

Coating

Diluted NH₂-Capture polymers to 0.1 μm in coating buffer

Added 100 μl/well and incubated at room temperature for 2 hours in the dark under agitation

1st Hybridization

Diluted analyte-binding oligonucleotide into lysis buffer

Prepared samples in lysis buffer

Washed plate with coat wash buffer (3 times)

Loaded 50 μl/well of capture hybridization buffer and 50 μl/well of sample in lysis buffer

Mixed gently and incubated overnight at 53° C. in the dark

2nd Hybridization

Cooled plates for 10 minutes at room temperature

Prepared labeled oligonucleotide in stem/labeled oligonucleotide buffer

Washed plate with wash buffer 1 (2 times)

Loaded 50 μl/well of labeled oligonucleotide solution

Incubated at 53° C. for 30 minutes

Label Detection

Cooled plates for 10 minutes at room temperature before washing with wash buffer 1 (2 times)

Prepared anti-label antibody in diluent and loaded 50 μl/well

Incubated at room temperature for 30 minutes under agitation and washed with wash buffer 1 (2 times) and 2 (3 times)

Added 50 μl/well of substrate solution and incubate at 37° C. for 1 hour before reading chemiluminescence

The analyte was human fetal hemoglobin provided as synthetic RNA. Synthetic fetal hemoglobin RNA was generated by in vitro transcription using a Megascript T7 kit (Cat. No. 1334, Ambion, Austin, Tex., USA). In brief, cloned hemoglobin DNA was inserted in Bluscript plasmid (Stratagene, La Jolla, Calif., USA) before being in vitro transcribed to generate the cRNA. Sequences of the capture polymers and analyte-binding labeled oligonucleotides are as described in Example 1.

Capture polymers were directly attached to assay plates according to the protocol as set forth above (coating step), using the DNA Immobilizer™ (Vedbaek, Denmark) plates according to manufacturer instructions. Capture polymers with an amino-group in their 3′ ends were generated and covalently attached to wells of the DNA Immobilizer microplate.

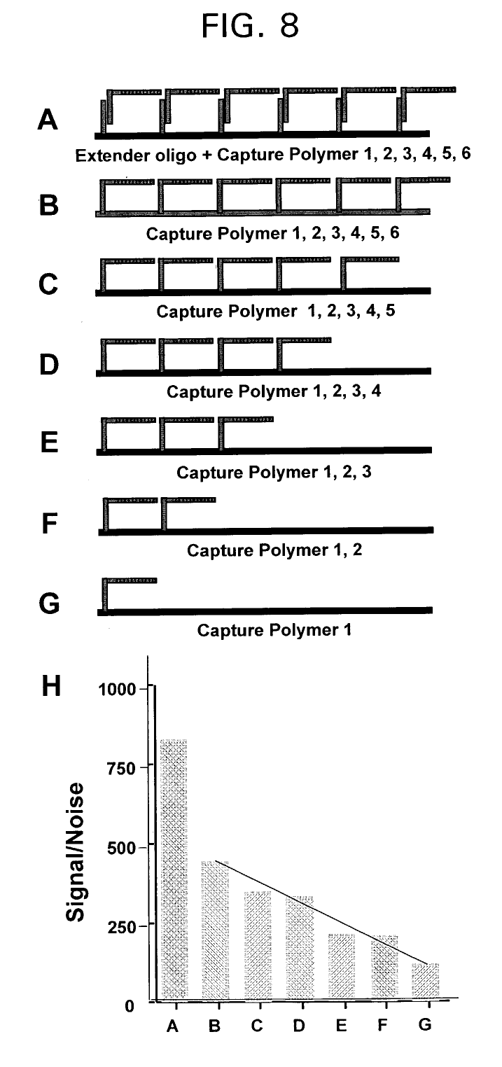

As shown in FIG. 8H, when capture polymers were directly attached to assay plates, the analyte was detectable and quantifiable, albeit with a reduced absolute signal/noise ratio value as compared to a method wherein capture polymers are indirectly attached to the assay plate. The greatly simplified nature of the direct attachment method nonetheless constitutes a significant advantage.

To test the effect on detection and quantitation capability of having more than one species of capture polymer in a single reaction, assays utilizing from one to six species of capture polymers in each assay well were performed. As shown in FIG. 8H, progressive reduction of the number of capture polymer species in each assay well resulted in a linear decrease in the values of signal/noise ratio. It should be noted, however, that even with one species of capture polymer, the analyte was detectable and quantifiable. The data demonstrate the flexibility of the present method.

Use of Capture Polymer Modified with C18 Spacer

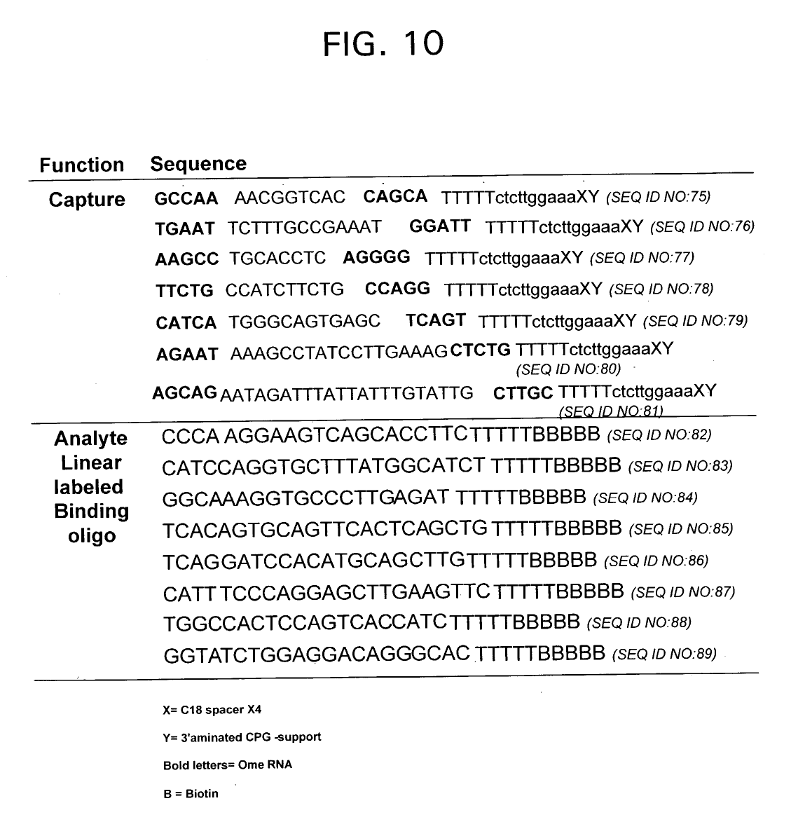

Capture polymers were also modified to remove regions that did not directly hybridize with the analyte. Four C18 spacers (Glen Research, Sterling, Va., USA) were introduced into the capture polymers to remove most of the sequences that were not intended to hybridize to the analyte. Sequences of these capture polymers, along with the analyte-binding linear labeled oligonucleotides used in conjunction with these capture polymers, are set forth in FIG. 10. These oligonucleotides and capture polymers were designed using the following algorithm:

Initialization:

find all exact repeats of 5 or longer, or 4 if 3GC

find potential complement regions

merge repeats if within 1 mis or 1 indel (2 mis if both are longer than 7)

retain those that have a Tm of 40

choose initial boundaries to cleave any retained complement regions if a complement region can't be split into two regions that have TM<40, reject the window

First Pass:

while room in window for another primer

find minimum primer that satisfies length and Tₘ

jump by length of primer

Second Pass:

while left over bases

increase length of shortest, recompute Tₘ for downstream

Third Pass:

check for any problem areas

juggle boundaries to minimize dimerization

The capture polymers, which had a 3′-ethylene glycol scaffolding and an amino-group attached to their 3′ ends, were covalently bound to assay plates as described above.

Analyte detection and quantitation was performed using the C18-modified capture polymers in conjunction with an analyte-binding linear labeled oligonucleotide according to the following reaction conditions:

Coating

Diluted NH₂-Capture polymers to 0.1 μm in coating buffer

Added 100 μl/well and incubated at room temperature for 2 hours in the dark under agitation

1st Hybridization

Diluted labeled analyte-binding linear labeled oligonucleotide into lysis buffer

Prepared samples in lysis buffer

Washed plate with coat wash buffer (3 times)

Loaded 50 μl/well of capture hybridization buffer and 50 μl/well of sample in lysis buffer

Mixed gently and incubated overnight at 53° C. in the dark

Label Detection

Cooled plates for 10 minutes at room temperature before washing with wash buffer 1 (2 times)

Prepared anti-label antibody in diluent and loaded 50 μl/well

Incubated at room temperature for 30 minutes under agitation and washed with wash buffer 1 (2 times) and 2 (3 times)

Added 50 μl/well of substrate solution and incubated at 37° C. for 1 hour before reading chemiluminescence

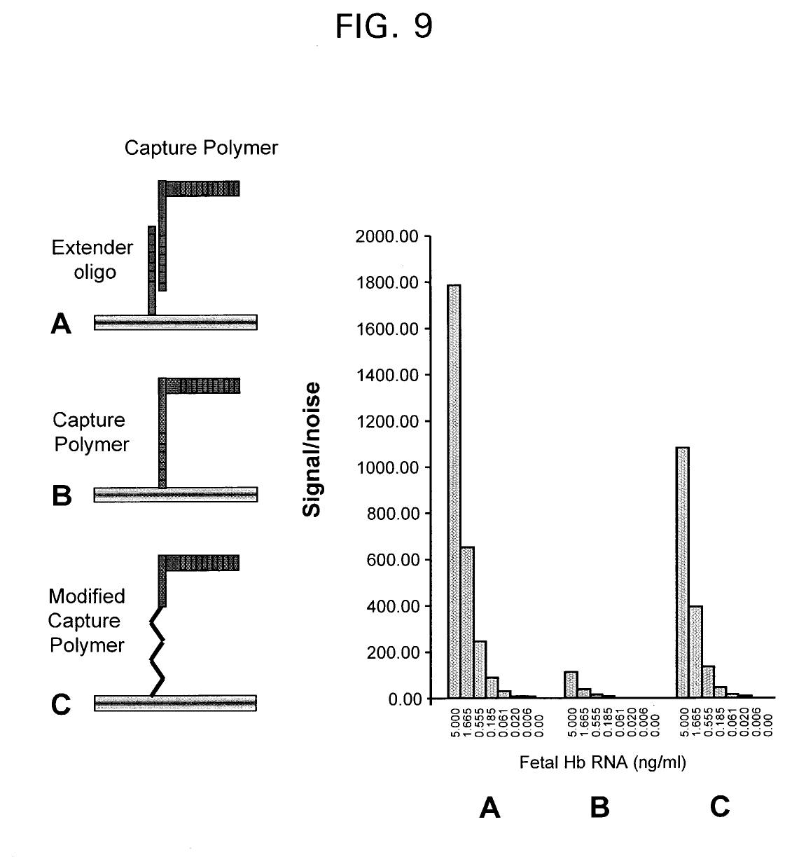

As shown in FIG. 9, introduction of the C18 (3′ethylene glycol) scaffolding into the capture polymers resulted in a substantial increase in detection and quantitation sensitivity (compare FIGS. 9B and 9C). Based on raw data (not shown), it was apparent that the use of the modified capture polymers resulted in a significant reduction of assay background (“noise”) without substantially affecting specific signal.

Use of Capture Polymer Modified with 2′-O-methoxy-RNA

C18-modified capture polymers were further modified by introduction of 2′-O-methoxy-RNA into the region of the capture polymer that was hybridizable to the analyte. 2′-O-methoxy-RNA are available from Glen Research (Sterling, Va., USA) and were introduced into capture polymers by standard phosphoramidite chemistry using an ABI DNA/RNA synthesizer. Capture polymers were generated with the sequence/configuration as set forth in FIG. 10, with the bold nucleotides being 2′-O-methoxy-RNA rather than dNTP. 2′-O-methoxy-RNA nucleotides were incorporated in the 3′ region of the capture polymer that was 5′ of the C-18 spacer scaffolding region.

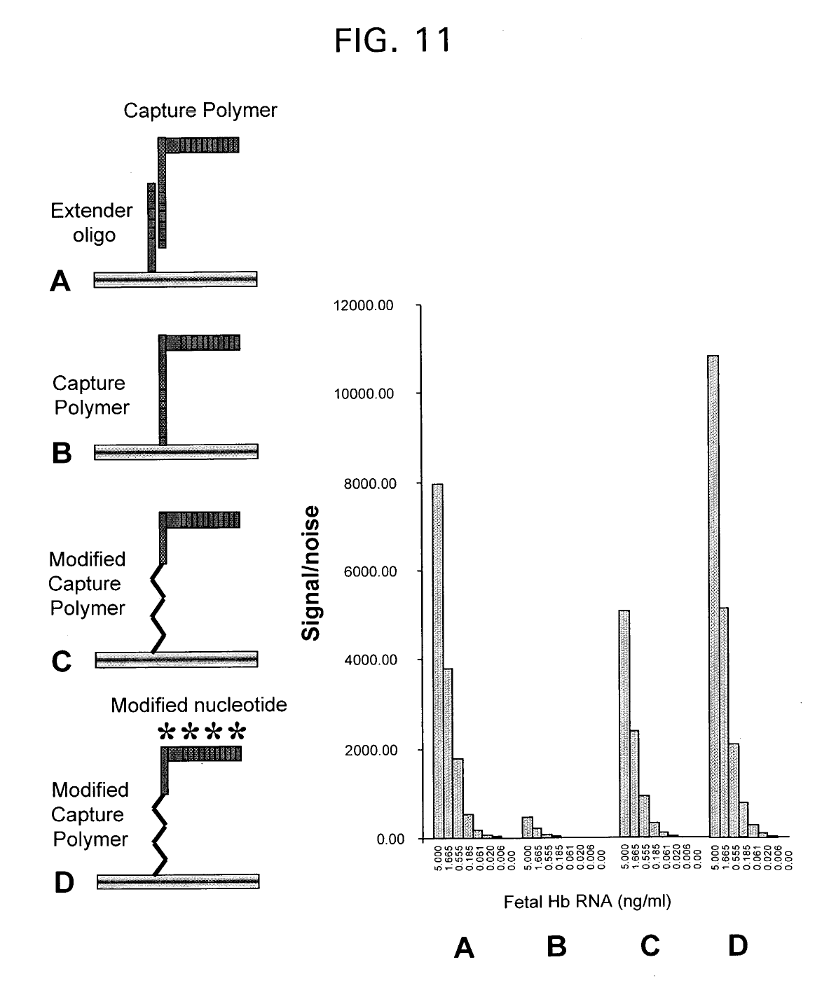

Analyte detection and quantitation was performed using the 2′-O-methoxy-RNA-modified capture polymers in conjunction with an analyte-binding linear labeled oligonucleotide according to the reaction conditions described above in the section captioned “Use of capture polymer modified with C18 spacer”. As shown in FIG. 11D, incorporation of 2′-O-methoxy-RNA resulted in a significant increase in signal/noise ratio compared to an unmodified capture polymer (FIG. 11B) and a C18-modified capture polymer (FIG. 11C). Indeed, the method using C-18- and 2′-O-methoxy-RNA-modified capture polymers provided noticeably better results than the method utilizing a capture polymer indirectly attached to the assay plate.

### Example 3

Detection and Quantitation of Analyte Using C18- and 2′-O-methoxy-RNA-modified Capture Polymers and Analyte-Binding Linear Labeled Oligonucleotide

Detection and quantitation of the human fetal hemoglobin RNA was performed by using C18- and 2′-O-methoxy-RNA-modified capture polymers and an analyte-binding oligonucleotide that was directly labeled (as illustrated in FIG. 6—referred to in this Example as “the direct & modified method”) in parallel with assays using unmodified capture polymers in conjunction with an analyte-binding oligonucleotide and a linear labeled oligonucleotide (as illustrated in FIG. 3—referred to in this Example as “the indirect & unmodified method”).

In the direct & modified method, capture polymers and the analyte-binding oligonucleotide targeting various contiguous regions on the human fetal hemoglobin RNA were designed using the algorithm in Example 2. Sequences of these oligonucleotides are as set forth in FIG. 10.

Modified capture polymers were covalently coated on DNA Immobilizer™ microplate (96 or 384 wells) according to the protocol described in Example 2. Reaction conditions and components are as described in Example 2.

The indirect & unmodified assay was performed according to the reaction conditions as described in Example 1. Sequences of the various components are as set forth in Example 1.

The labeled oligonucleotides were labeled with either digoxigenin, FITC or biotin, and detected with the corresponding alkaline phosphatase-conjugated antibodies according to the reaction conditions set forth above.

Determination of Effects of Source of Alkaline Phosphatase Substrate for Label Detection and Generation of Signal

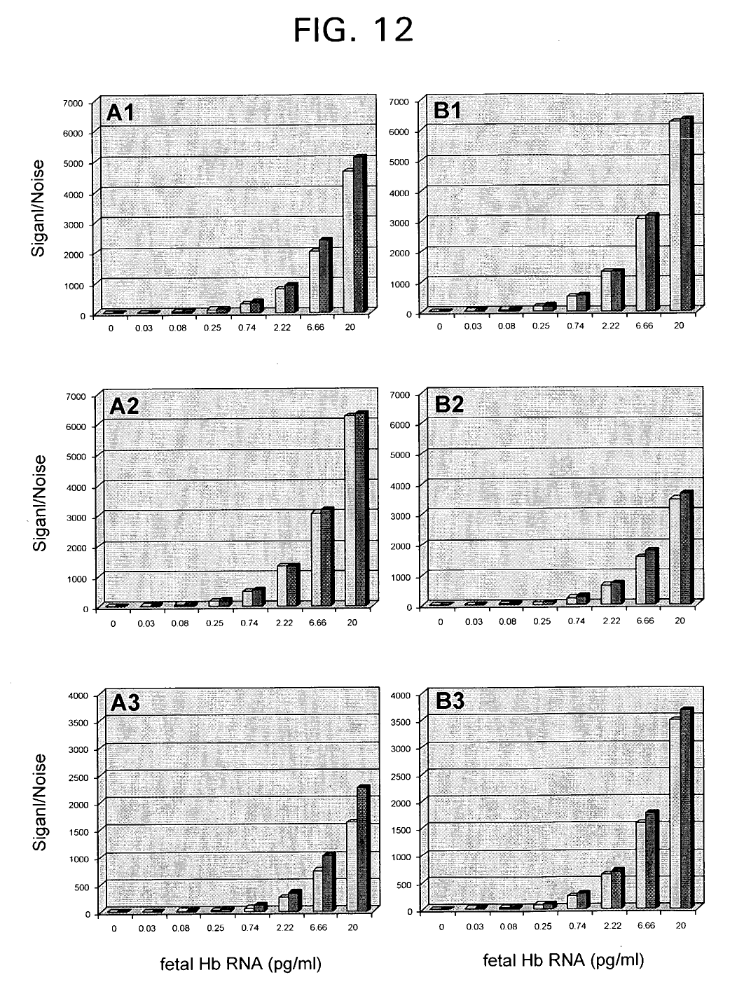

For generation of detectable signal, alkaline phosphatase substrates from different sources were tested in accordance with manufacturer instructions. As shown in FIGS. 12A1 & B1, signal/noise ratios generated using either the Bold 540 (Intergen, Purchase, N.Y., USA) or CDP-Star (InnoGenex, San Ramon, Calif., USA) substrates were comparable across a wide range of analyte concentrations. It was notable, however, that the values generated using CDP-Star appeared to be higher (in some cases by about 50%) compared with those generated using Bold 540. Also, the sensitivity of the direct & modified method was on average better than that of the indirect & unmodified method.

Determination of Effects of Signal Reader on Assay Performance

To determine the effect, if any, of choice of signal (plate) reader, two different readers were tested to read the luminescent signal generated. As shown in FIGS. 12A2 & B2, detection and quantitation was achieved using either the Dynex MLX Microplate luminometer (Dynex, Chantilly, Va., USA) or the Victor2 V, 1420 Multilabel HTS counter (Wallac/Perkin Elmer, Shelton, Conn., USA). It is notable, however, that the values generated using the Dynex reader were generally higher compared to those generated with the Wallac reader. Also, the sensitivity of the direct & modified method was on average better than that of the indirect & unmodified method.

Determination of Effects of Plate Format on Assay Performance

Assays were performed on both 384-well and 96-well plates. As shown in FIGS. 12A3 & B3, detection and quantitation was achieved when either plate formats was used. Indeed, with the 96-well format, there was a ten-fold increase in signal compared to noise with less than 0.03 pg/ml of analyte, and with the 384-well format, there was a more than four fold increase in signal compared to noise with less than 0.03 pg/ml of analyte. Also, the sensitivity of the direct & modified method was on average better than that of the indirect & unmodified method.

### Example 4

Detection and Quantitation of Human Fc mRNA

Detection and quantitation of human Fc mRNA was performed by using either C18- and 2′-O-methoxy-RNA-modified capture polymers and an analyte-binding oligonucleotide that was indirectly labeled through hybridization with a linear labeled oligonucleotide (referred to in this Example as “Method 1”) or C18- and 2′-O-methoxy-RNA-modified capture polymers and an analyte-binding oligonucleotide that was directly labeled (referred to in this Example as “Method 2”).

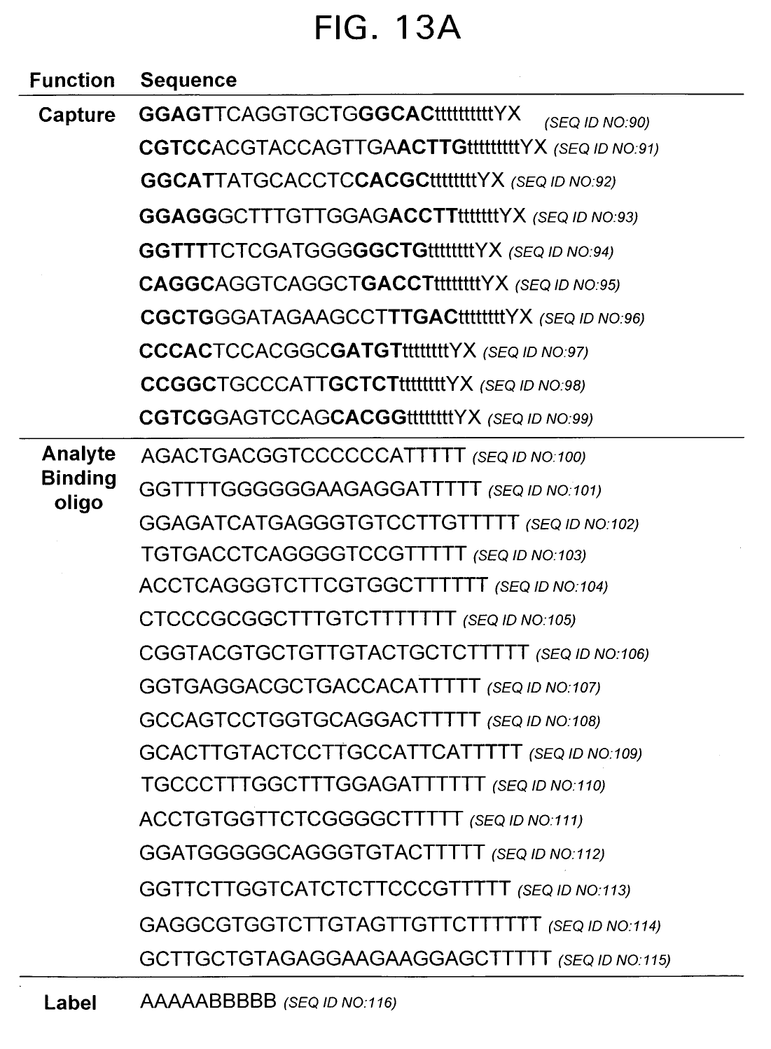

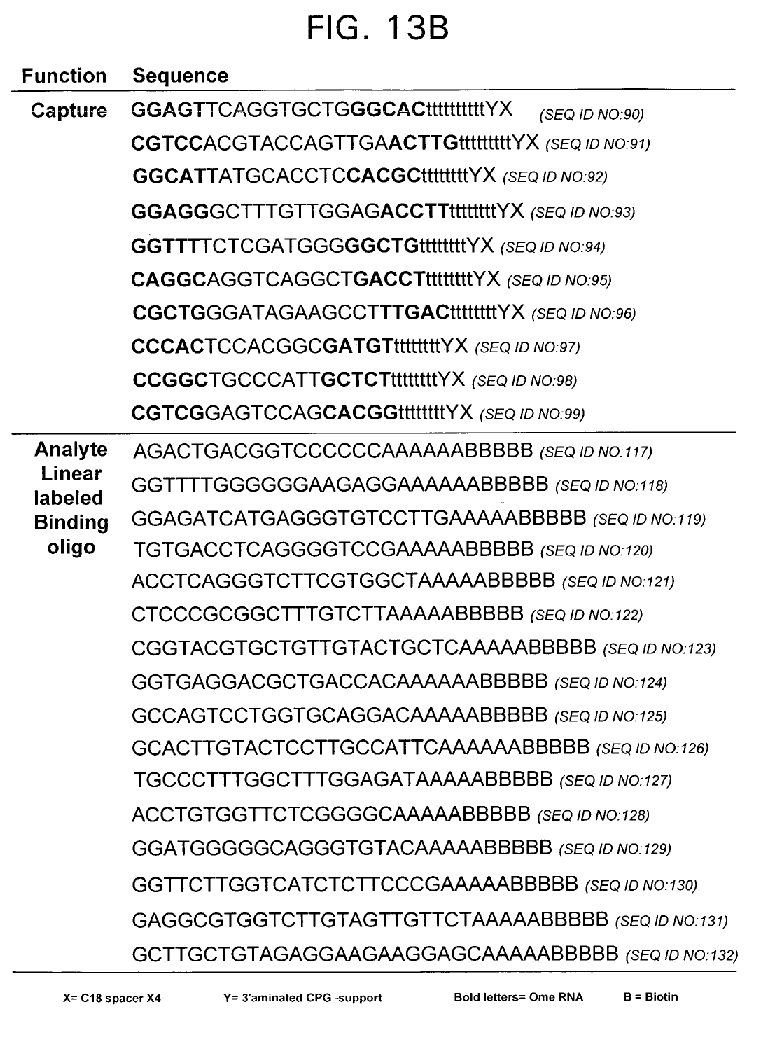

Sequences of the oligonucleotides used are as set forth in FIGS. 13A & B. Ten species of capture polymers were provided in each assay well.

Capture polymers were covalently coated on DNA Immobilizer™ microplate (96 wells) according to the protocol described in Example 2. Reaction conditions and components were as described in Example 2.

The labeled oligonucleotides (whether in the analyte-binding labeled oligonucleotide or the linear labeled oligonucleotide that hybridizes to the analyte-binding oligonucleotide) were labeled with biotin, and detected with the biotinylated alkaline phosphatase-conjugated antibodies according the reaction conditions set forth above.

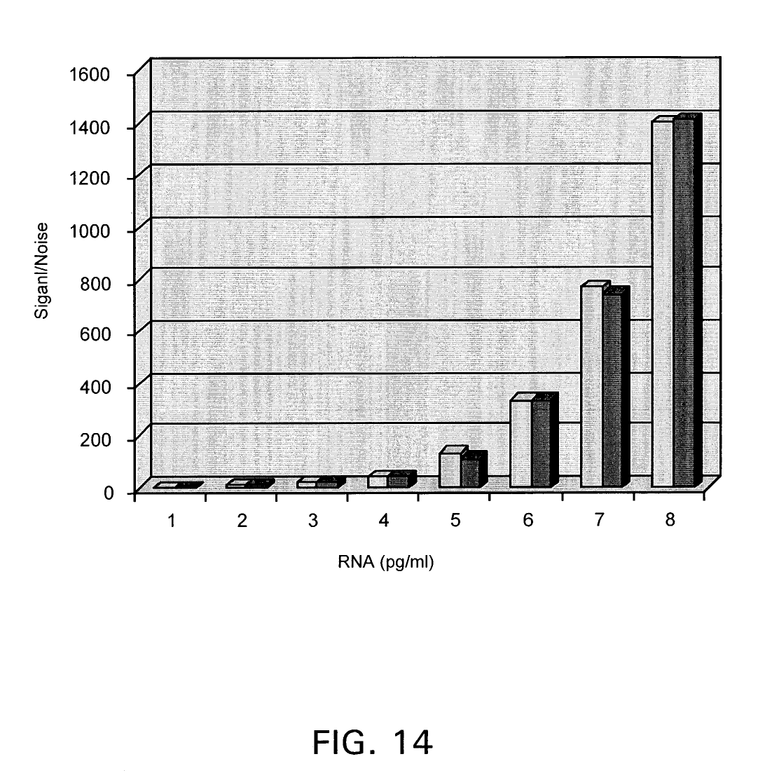

As shown in FIG. 14, similar detection and quantitation performance was observed with both methods (1 & 2). Thus, the methods of the invention are capable of detecting and quantitating in a sequence-independent manner (i.e., it is not specific to just one gene/sequence). Furthermore, the analyte-binding oligonucleotide may be directly or indirectly labeled, as desired by the practitioner, without any substantial loss of assay sensitivity.

### Example 5

Application of the Directly Attached Capture Polymer Format in an Array System

The ability to detect and quantitate analytes using the method of the invention wherein capture polymers are directly attached to a solid support provides advantageous flexibility for adaptation of the method to an array format. To test this observation, C18- and 2′-O-methoxy-RNA-modified capture polymers with the sequence set forth in FIG. 10 were used. These capture polymers comprised 2′-O-methoxy-RNA in the 3′ and 5′ region of the sequence that is hybridizable to the analyte as well as C18 ethylene glycol scaffolding in the 3′ region of the capture polymer and a 3′-end amino group. The capture polymers were directly attached to plates as described above in Examples 2-4.

Human fetal hemoglobin cDNAs were directly labeled with digoxigenin using a digoxigenin oligonucleotide tailing kit (Roche Molecular Biochemicals, Cat. No. 1-417-231, Indianapolis, Ind., USA) according to manufacturer instructions. Various amounts of labeled cDNA were then hybridized onto the coated 96-well plates. Reaction buffers were as described in Example 1. Reaction conditions were as follows:

Coating

Diluted NH₂-Capture polymers to 0.1 μm in coating buffer

Added 100 μl/well and incubated at room temperature for 2 hours in the dark under agitation

Array Application

1st Hybridizatioin

Washed plate with coat wash buffer (3 times)

Loaded 50 μl/well of capture hybridization buffer and 50 μl/well of DIG-labeled sample

Mixed gently and incubated overnight at 53° C. in the dark

Label Detection

Cooled plates for 10 minutes at room temperature before washing with wash buffer 1 (2 times)

Prepared anti-label antibody in diluent and loaded 50 μl/well

Incubated at room temperature for 30 minutes under agitation and washed with wash buffer 1 (2 times) and 2 (3 times)

Added 50 μl/well of substrate solution and incubated at 37° C. for 1 hour before reading chemiluminescence

Captured cDNA was detected by contacting the complex formed with an anti-digoxigenin antibody conjugated to alkaline phosphase using CDP-Star substrate as described in Example 3 above. Signal was measured with a MLX microplate luminometer (Dynex, Chantilly, Va., USA).

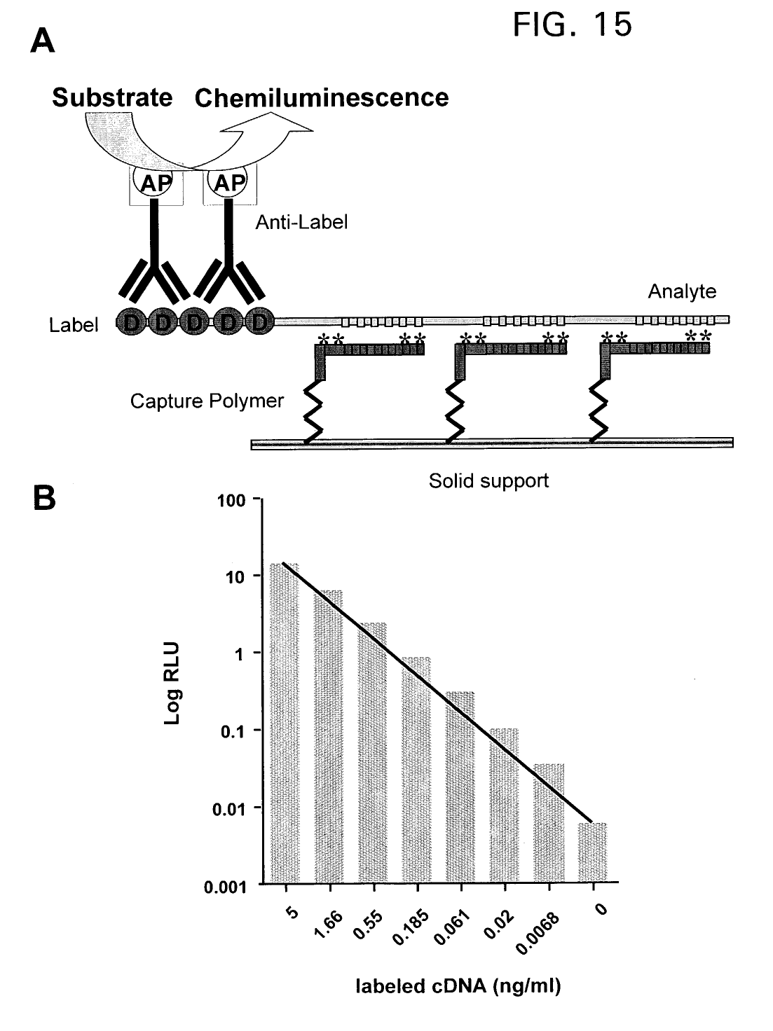

As shown in FIG. 15, a linear signal was obtained between 0 and 5 ng/ml of human fetal hemoglobin cDNA with this DNA array format. Assay sensitivity also appeared to be good, with a two-fold background signal observed with 6.8 pg/ml of cDNA.

### Example 6

Use of Methods of the Invention in High throughput Cellular Clone Selection Process

Generating cell lines with high specificity and productivity is a labor-intensive process with limited throughput. As part of this process, hundreds of clones are screened for specific productivity using human Fc immunoassay and cell count data. However, the need to set up multiple cell plates and to perform multiple sample dilutions from these plates significantly decrease the throughput of this traditional method. Since mRNA levels generally correlate with specific productivity, the use of methods of the invention to streamline the cellular clone selection process was investigated.

Our results showed that detection and quantitation of human Fc by methods of the invention can be used to support high throughput clone screening without the need for multiple sampling days and RNA extraction, allowing for thousands of clones to be rapidly screened for productivity. As described below, the invention can be used to analyze Fc mRNA level in cell lines expressing different recombinant antibodies, with a linear correlation between methods of the invention and specific productivity assays traditionally used for clone screening. In addition, unlike specific productivity assays, methods of the invention are capable of providing an accurate ranking of the clones using a single sampling time point. The data demonstrate that methods of the invention can support high throughput clone screening during the development of commercial production cell lines.

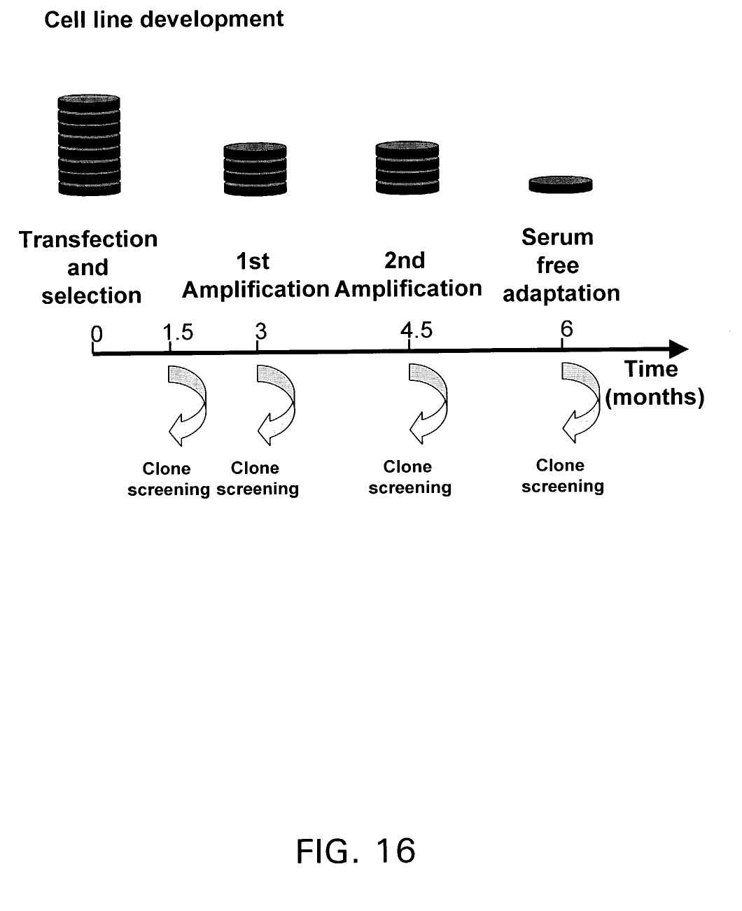

FIG. 16 schematically illustrates an embodiment of a cell line development process. During the generation of production cell lines hundreds of clones are screened for specific productivity to select for cell lines with high specific and volumetric productivity. During the development of these cell lines, multiple measurements are performed in order to select the best producers. Typically, levels of recombinant proteins produced are assessed in the conditioned media using specific immunoassay and cell count data. The moderate throughput of this approach results from the need to set up multiple culture plates for analysis at different days of culture as well as performing several sample dilutions. Considering the good correlation between specific productivity and mRNA levels, we decided to investigate the use of a method of the invention for clone selection with the ultimate goal of increasing clone screening capacity.

Validation of Method of the Invention as a Useful and Superior Quantitation Tool for Production Cell Line Clone Selection

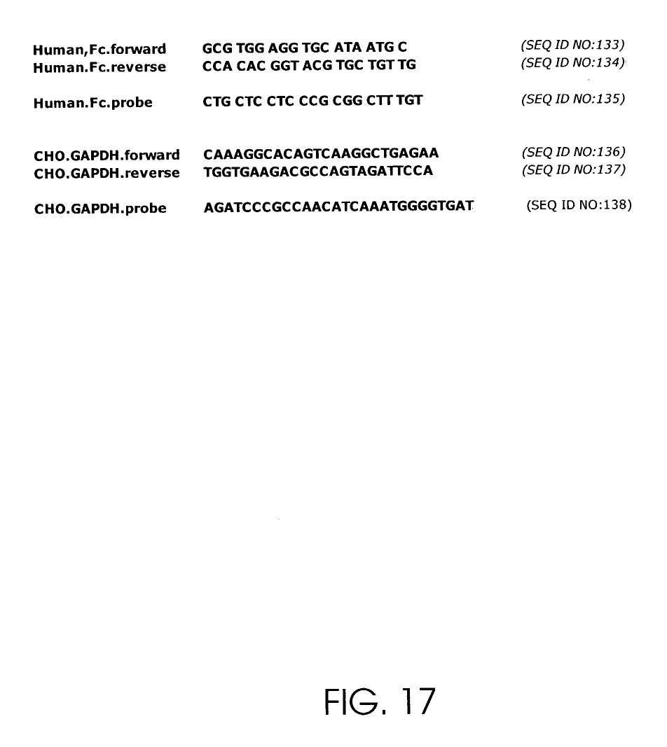

Fourteen different CHO (Chinese Hamster Ovary) cell clones producing a recombinant human monoclonal antibody were seeded in 96-well plates at a density between 1×10³ and 80×10³ cells/well with 100 ul culture media. Cell number was assessed using a Z2 cell coulter (Beckman Coulter) before seeding and using the Alamar Blue (Biosource International, Inc., Camarillo, Calif., USA) or Calcein-AM (Molecular Probes, Inc., Eugene, Oreg., USA) fluorescent readout after culture. Conditioned media was collected to measure human IgG concentrations using an intact IgG immunoassay and cells were subsequently lysed using a lysis buffer (1 M HEPES, pH 8.0; 10% lithium lauryl sulfate; 0.25 M EDTA; 5M lithium chloride; 600 mg/liter Proteinase K; Micro-protect (Boehringer Manheim)). Detection and quantitation was performed as follows: Extender oligonucleotides (sequences as provided in FIG. 7) were synthesized with an amino-group at the 3′ end for covalent coupling to 96-well DNA Immobilizer™ plates (Exiqon) as described in the Examples above. Human Fc mRNA-specific capture polymers (sequence as provided in FIG. 13A) and analyte-binding oligonucleotides (sequence as provided in FIG. 13A) were added to the transfected CHO cell lysates (⅓ and 1/9 dilution) before mixing with capture hybridization buffer (6×SSC buffer (Sodium chloride/Sodium Citrate); 0.1% SDS; 50 mg/ml salmon sperm DNA) and loading onto the coated DNA Immobilizer™ plates. Hybridization occurred overnight at 53° C. in the dark. The next day, the plates were cooled to room temperature and washed with wash buffer (0.1×SSC buffer; 0.1% SDS). Diluted stem oligonucleotides (sequence as provided in FIG. 7) (conditions as described in Example 1) in label buffer (6×SSC; 10% BM block (Boehringer Mannheim) was then added before incubating at 53° C. for 30 minutes. After cooling to room temperature again, the plates were washed with wash buffer before the addition of the digoxigenin-labeled oligonucleotide (sequence as set forth in FIG. 7). After another 30 minutes of incubation at 53° C., the plates were cooled back down to room temperature. Then plates were washed with wash buffer and incubated at room temperature for 30 minutes in the presence of anti-digoxigenin antibody conjugated to alkaline phosphatase. Finally, the plates were treated with alkaline phosphatase substrate (CDP-Star (InnoGenex, San Ramon, Calif., USA) or Bold 540; Intergen, Purchase, N.Y., USA) and incubated at 37° C. for 15-30 minutes. The chemiluminescence was read using an MLX Microplate luminometer (Dynex). Quantitative RT-PCR Taqman analysis was also run on the cell lysates using a human Fc specific set of primers and probe. Samples were also analyzed using primers and probe specific to GAPDH to provide reference data for normalization of Fc data. RT-PCR conditions were as follows: 1 cycle at 48° C. for 30 min., followed by 1 cyle at 95° C. for 10 min., followed by 40 cycles consisting of alternating between 95° C. (20 sec) and 60° C. (60 sec); ending with 1 cycle at 25° C. (2 min). Primer and probe sequences were as set forth in FIG. 17.

Intact IgG ELISA was performed with materials and methods as follows:

Materials

| | | |
|---|---|---|
| 1. | Solid support: | Nunc immunoplate (Nunc catalog no. 4-39454) |
| 2. | Coating buffer: | 0.05 M Carbonate/bicarbonate, pH 9.6 |
| 3. | Washing buffer: | PBS + 0.05% Tween 20 |
| 4. | Blocking buffer: | PBS + 0.5% BSA + 0.01% Thimerosal pH 7.4 |
| 5. | Assay buffer: | PBS + 0.5% BSA + 0.05% Tween 20 + |
| | | 10 ppm Proclin, pH 7.4 |
| 6. | Coat Antibody: | Goat anti-human IgG Fab |
| | | Source: Cappel Cat #109-005-097 ; 1.8 mg/mL |
| 7. | Standard: | rhuMAb HFR2 (stock concentration = 10 ug/ml) |
| | | (Genentech, South San Francisco) |
| 8. | Conj. Antibody: | Goat Anti-hu IgG Fc-HRP Cappel Cat #55253 |
| 9. | Substrate: | TMB (Moss, Pasadena, MD, USA; Product |
| | | Number TMBE 1000) |
| 10. | Stopping Soln: | 1 M Phosphoric Acid |

Procedure

Coating

1) Dilution of coat:

| | Concentration | Final | Dilution required |
|---|---|---|---|
| Antibody | (mg/ml) | conc. | 1: |
| Gt-anti-hu | 1.8 | 2 ug/ml | 900 |
| IgG Fab | | | |

2) Added 100 μl of diluted antibody of (1) to each well and coated overnight at 4° C.

3) Discarded the antibody from (2) and added 150 μl of blocking buffer to each well.

4) Incubated for 1 hr at R.T. w/gentle agitation.

Assay

1) Preparation of standard:

Prepared 200 ng/ml standard from stock of 10 ug/ml using a 1:50 dilution. Did 1:2.5 serial dilutions to go from 200 ng/ml down to 0.33 ng/ml.

2) Added 100 ul of standards and samples (conditioned culture media as noted above) into appropriate wells.

3) Incubated for 1 hour at room temperature (RT).

4) Washed plates 3× with washing buffer.

5) Prepared conjugated antibody (assay concentration 175 pg/mL)

6) Washed plates 3× with washing buffer.

7) Added 100 ul of conjugated antibody to each well.

8) Incubated for 1 hour at room temperature.

Washed plates 3× with wash buffer.

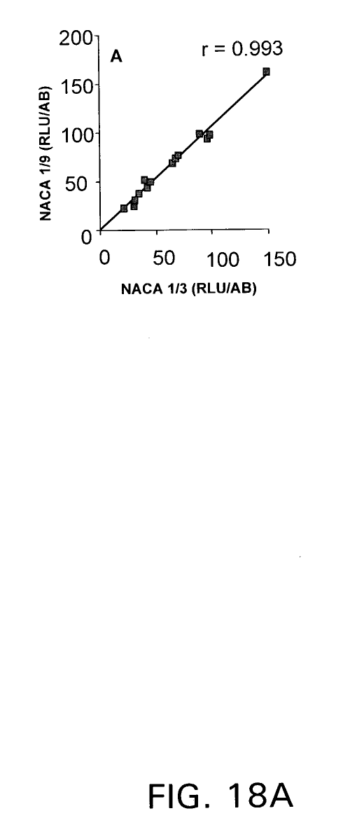

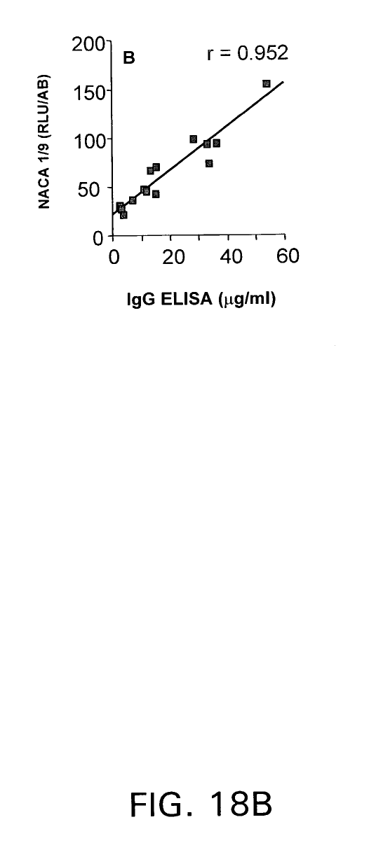

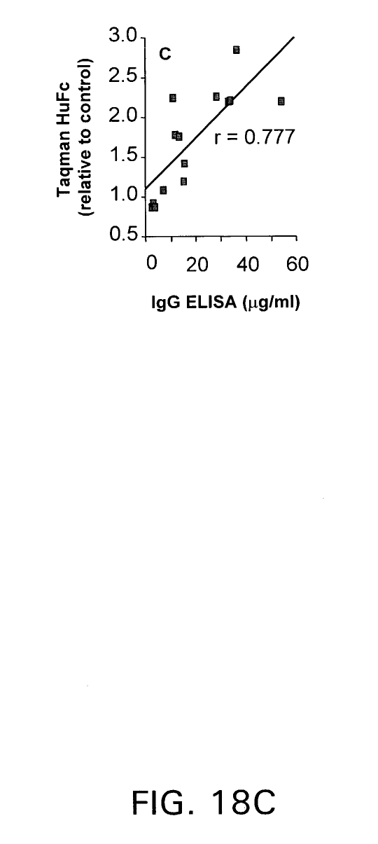

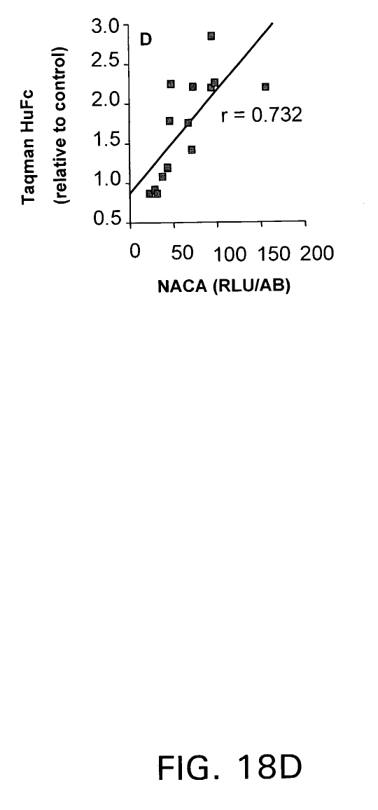

FIG. 18 shows results demonstrating the following:

FIG. 18A: Human Fc data from quantitation using method of the invention as described above in cell lysate samples diluted ⅓ and 1/9. Data from ⅓ dilutions correlate with data obtained using cell lysates diluted 1/9, demonstrating the linearity of the method of the invention in these conditions.

FIG. 18B: Data obtained by method of the invention as described above correlate well with the intact human IgG immunoassay data, confirming the correlation between specific productivities and mRNA levels. This result also validates methods of the invention as reliable tools for screening production cell clones.

FIG. 18C: Human Fc mRNA levels as determined by RT-PCR Taqman correlate well with amounts of human IgG protein measured using the intact IgG immunoassay.

FIG. 18D: Human Fc data obtained by method of the invention as described above correlate with Taqman data similarly to intact IgG immunoassay data (FIG. 18C). This validates the approach of using a method of the invention for production cell line clone screening purposes, as compared to a screening strategy based on a well-established gene expression analysis method (RT-PCR Taqman).

Comparison of Method of the Invention and IgG ELISA for Detection/Quanititation of Human Fc at Different Cell Culture Sampling Time Points

Various CHO clones producing a recombinant human monoclonal antibody were analyzed using method of the invention as described above as well as the intact IgG immunoassay (also as described above). The conventional intact IgG immunoassay requires several sample dilution points, as well as multiple sampling time points to ensure accurate ranking of different clones. In this analysis, detection of antibody production was assessed using method of the invention as described above in conditioned media and cell lysate collected after 2 and 3 days of culture. At both time points, data obtained by method of the invention as described above highly correlated with specific productivity (r>0.97) as assessed by intact IgG ELISA as described above (data not shown). This demonstrates that unlike the conventional immunoassay method, a single time point can be used to accurately rank clones using the invention method. Thus, methods of the invention provide superior advantages that could significantly improve the throughput level of production cell clone screening strategies.

Applicability of Methods of the Invention for Cell Clone Screening for a Variety of Cell Lines

The correlation between intact IgG immunoassay data and data obtained by methods of the invention was assessed across various cell lines and cell clones. A good correlation between data obtained by these two methods was observed (r>0.8), demonstrating the applicability of methods of the invention for screening a large number of clones for cell lines expressing a variety of recombinant antibodies.

Thus, as the data described above demonstrate, there is good correlation between mRNA levels (as determined using methods of the invention) and specific productivity (protein levels determined using ELISA). Methods of the invention are demonstrably reliable, useful and superior for use in efficiently screening a large number of cell clones in a manner that is independent of the specific antibody produced by the cells. Using methods of the invention, fewer dilution points are needed, and only a single sample time point is necessary, thus enabling automation and higher throughput in a process that has traditionally been laborious, inefficient and costly.

1. A method for detecting or quantitating a nucleic acid analyte in a sample, said method comprising:
(A) contacting said sample with an analyte-binding oligonucleotide, a labeled oligonucleotide, a capture polymer and a linear stem oligonucleotide under conditions whereby a complex is formed comprising the analyte, analyte-binding oligonucleotide, labeled oligonucleotide, capture polymer and linear stem oligonucleotide, wherein:
(i) the analyte-binding oligonucleotide comprises (a) a sequence that is hybridizable to the analyte and (b) a sequence that is hybridizable to the stem oligonucleotide;
(ii) the linear stem oligonucleotide comprises (a) a sequence that is hybridizable to the analyte-binding oligonucleotide and (b) a sequence that is directly or indirectly hybridizable to the labeled oligonucleotide;
(iii) the labeled oligonucleotide comprises (a) a sequence that is directly or indirectly hybridizable to the stem oligonucleotide and (b) a label capable of directly or indirectly generating a detectable signal;
(iv) the capture polymer comprises a nucleic acid sequence that is directly or indirectly hybridizable to the analyte;
(B) detecting or quantitating the complex of step (A);
whereby detection or quantitation of the complex of step (A) is indicative of presence or quantity of the nucleic acid analyte in the sample.

2. A method for detecting or quantitating a nucleic acid analyte in a sample, said method comprising:
(A) contacting said sample with an analyte-binding oligonucleotide, a linear labeled oligonucleotide and a capture polymer under conditions whereby a complex is formed comprising the analyte, analyte-binding oligonucleotide, linear labeled oligonucleotide and capture polymer, wherein:
(i) the analyte-binding oligonucleotide comprises (a) a sequence that is hybridizable to the analyte and (b) a sequence that is hybridizable to the linear labeled oligonucleotide;
(ii) the linear labeled oligonucleotide comprises (a) two or more units of label each attached directly to the oligonucleotide and (b) a sequence that is hybridizable to the analyte-binding oligonucleotide;
(iii) the capture polymer comprises a nucleic acid sequence that is directly or indirectly hybridizable to the analyte;
(B) detecting or quantitating the complex of step (A);
whereby detection or quantitation of the complex of step (A) is indicative of presence or quantity of the nucleic acid analyte in the sample.

3. A method for detecting or quantitating a nucleic acid analyte in a sample, said method comprising:
(A) contacting said sample with an analyte-binding linear labeled oligonucleotide and a capture polymer under conditions whereby a complex is formed comprising the analyte, analyte-binding linear labeled oligonucleotide and capture polymer, wherein:
(i) the analyte-binding linear labeled oligonucleotide comprises (a) a sequence that is hybridizable to the analyte and (b) two or more units of label each attached directly to the oligonucleotide;
(ii) the capture polymer comprises a nucleic acid sequence that is directly or indirectly hybridizable to the analyte;
(B) detecting or quantitating the complex of step (A);
whereby detection or quantitation of the complex of step (A) is indicative of presence or quantity of the nucleic acid analyte in the sample.

4. A method for detecting or quantitating a nucleic acid analyte in a sample, said method comprising:
(a) contacting the sample with an analyte-binding oligonucleotide and a capture polymer under conditions whereby a complex is formed comprising the analyte, analyte-binding oligonucleotide, and capture polymer, wherein:
(i) the analyte-binding oligonucleotide comprises a sequence that is hybridizable to the analyte; and
(ii) the capture polymer comprises a first portion that is hybridizable to the analyte and a second portion comprising a material that is not substantially hybridizable to nucleic acid;
(b) detecting or quantitating the complex of step (a);
whereby detection or quantitation of the complex of step (a) is indicative of presence or quantity of the nucleic acid analyte in the sample.

5. A method for detecting or quantitating a nucleic acid analyte in a sample, said method comprising:
(a) contacting the sample with an analyte-binding oligonucleotide and a capture polymer under conditions whereby a complex is formed comprising the analyte, analyte-binding oligonucleotide, and capture polymer, wherein:
(i) the analyte-binding oligonucleotide comprises a sequence that is hybridizable to the analyte; and
(ii) the capture polymer comprises a sequence that is hybridizable to the analyte and further comprises at least one modified nucleotide that enhances strength of hybridization of the polymer to the analyte;
(b) detecting or quantitating the complex of step (a);
whereby detection or quantitation of the complex of step (a) is indicative of presence or quantity of the nucleic acid analyte in the sample.

6. A method for detecting or quantitating a nucleic acid analyte in a sample, said method comprising:
(a) contacting the sample with an analyte-binding oligonucleotide and a capture polymer under conditions whereby a complex is formed comprising the analyte, analyte-binding oligonucleotide, and capture polymer, wherein:
(i) the analyte-binding oligonucleotide comprises a sequence that is hybridizable to the analyte; and
(ii) the capture polymer comprises a first portion that is hybridizable to the analyte, said first portion comprising at least one modified nucleotide that enhances strength of hybridization of the polymer to the analyte, and a second portion comprising a material that is not substantially hybridizable to nucleic acid;
(b) detecting or quantitating the complex of step (a);
whereby detection or quantitation of the complex of step (a) is indicative of presence or quantity of the nucleic acid analyte in the sample.

7. A method for detecting or quantitating a nucleic acid analyte in a sample, said method comprising:
(A) contacting said sample with an analyte-binding oligonucleotide, a labeled oligonucleotide, a capture polymer and a linear stem oligonucleotide under conditions whereby a complex is formed comprising the analyte, analyte-binding oligonucleotide, labeled oligonucleotide, capture polymer and linear stem oligonucleotide, wherein:
(i) the analyte-binding oligonucleotide comprises (a) a sequence that is hybridizable to the analyte and (b) a sequence that is hybridizable to the stem oligonucleotide;
(ii) the linear stem oligonucleotide comprises (a) a sequence that is hybridizable to the analyte-binding oligonucleotide and (b) a sequence that is directly or indirectly hybridizable to the labeled oligonucleotide;
(iii) the labeled oligonucleotide comprises (a) a sequence that is directly or indirectly hybridizable to the stem oligonucleotide and (b) a label capable of directly or indirectly generating a detectable signal;
(iv) the capture polymer comprises a first portion that is hybridizable to the analyte and and a second portion comprising a material that is not substantially hybridizable to nucleic acid;
(B) detecting or quantitating the complex of step (A);
whereby detection or quantitation of the complex of step (A) is indicative of presence or quantity of the nucleic acid analyte in the sample.

8. A method for detecting or quantitating a nucleic acid analyte in a sample, said method comprising:
(A) contacting said sample with an analyte-binding oligonucleotide, a linear labeled oligonucleotide and a capture polymer under conditions whereby a complex is formed comprising the analyte, analyte-binding oligonucleotide, linear labeled oligonucleotide and capture polymer, wherein:
(i) the analyte-binding oligonucleotide comprises (a) a sequence that is hybridizable to the analyte and (b) a sequence that is hybridizable to the linear labeled oligonucleotide;
(ii) the linear labeled oligonucleotide comprises (a) two or more units of label each attached directly to the oligonucleotide and (b) a sequence that is hybridizable to the analyte-binding oligonucleotide;
(iii) the capture polymer comprises a first portion that is hybridizable to the analyte and a second portion comprising a material that is not substantially hybridizable to nucleic acid;
(B) detecting or quantitating the complex of step (A);
whereby detection or quantitation of the complex of step (A) is indicative of presence or quantity of the nucleic acid analyte in the sample.

9. A method for detecting or quantitating a nucleic acid analyte in a sample, said method comprising:
(A) contacting said sample with an analyte-binding linear labeled oligonucleotide and a capture polymer under conditions whereby a complex is formed comprising the analyte, analyte-binding linear labeled oligonucleotide and capture polymer, wherein:
(i) the analyte-binding linear labeled oligonucleotide comprises (a) a sequence that is hybridizable to the analyte and (b) two or more units of label each attached directly to the oligonucleotide;
(ii) the capture polymer comprises a first portion that is hybridizable to the analyte and a second portion comprising a material that is not substantially hybridizable to nucleic acid;
(B) detecting or quantitating the complex of step (A);
whereby detection or quantitation of the complex of step (A) is indicative of presence or quantity of the nucleic acid analyte in the sample.

10. A method for detecting or quantitating a nucleic acid analyte in a sample, said method comprising:
(A) contacting said sample with an analyte-binding oligonucleotide, a labeled oligonucleotide, a capture polymer and a linear stem oligonucleotide under conditions whereby a complex is formed comprising the analyte, analyte-binding oligonucleotide, labeled oligonucleotide, capture polymer and linear stem oligonucleotide, wherein:
(i) the analyte-binding oligonucleotide comprises (a) a sequence that is hybridizable to the analyte and (b) a sequence that is hybridizable to the stem oligonucleotide;
(ii) the linear stem oligonucleotide comprises (a) a sequence that is hybridizable to the analyte-binding oligonucleotide and (b) a sequence that is directly or indirectly hybridizable to the labeled oligonucleotide;
(iii) the labeled oligonucleotide comprises (a) a sequence that is directly or indirectly hybridizable to the stem oligonucleotide and (b) a label capable of directly or indirectly generating a detectable signal;
(iv) the capture polymer comprises a nucleic acid sequence that is hybridizable to the analyte and further comprises at least one modified nucleotide that enhances strength of hybridization of the polymer to the analyte;
(B) detecting or quantitating the complex of step (A);
whereby detection or quantitation of the complex of step (A) is indicative of presence or quantity of the nucleic acid analyte in the sample.

11. A method for detecting or quantitating a nucleic acid analyte in a sample, said method comprising:
(A) contacting said sample with an analyte-binding oligonucleotide, a linear labeled oligonucleotide and a capture polymer under conditions whereby a complex is formed comprising the analyte, analyte-binding oligonucleotide, linear labeled oligonucleotide and capture polymer, wherein:
(i) the analyte-binding oligonucleotide comprises (a) a sequence that is hybridizable to the analyte and (b) a sequence that is hybridizable to the linear labeled oligonucleotide;
(ii) the linear labeled oligonucleotide comprises (a) two or more units of label each attached directly to the oligonucleotide and (b) a sequence that is hybridizable to the analyte-binding oligonucleotide;
(iii) the capture polymer comprises a nucleic acid sequence that is hybridizable to the analyte and further comprises at least one modified nucleotide that enhances strength of hybridization of the polymer to the analyte;
(B) detecting or quantitating the complex of step (A);
whereby detection or quantitation of the complex of step (A) is indicative of presence or quantity of the nucleic acid analyte in the sample.

12. A method for detecting or quantitating a nucleic acid analyte in a sample, said method comprising:
(A) contacting said sample with an analyte-binding linear labeled oligonucleotide and a capture polymer under conditions whereby a complex is formed comprising the analyte, analyte-binding linear labeled oligonucleotide and capture polymer, wherein:
(i) the analyte-binding linear labeled oligonucleotide comprises (a) a sequence that is hybridizable to the analyte and (b) two or more units of label each attached directly to the oligonucleotide;
(ii) the capture polymer comprises a nucleic acid sequence that is hybridizable to the analyte and further comprises at least one modified nucleotide that enhances strength of hybridization of the polymer to the analyte;
(B) detecting or quantitating the complex of step (A);
whereby detection or quantitation of the complex of step (A) is indicative of presence or quantity of the nucleic acid analyte in the sample.

13. A method for detecting or quantitating a nucleic acid analyte in a sample, said method comprising:
(A) contacting said sample with an analyte-binding oligonucleotide, a labeled oligonucleotide, a capture polymer and a linear stem oligonucleotide under conditions whereby a complex is formed comprising the analyte, analyte-binding oligonucleotide, labeled oligonucleotide, capture polymer and linear stem oligonucleotide, wherein:
(i) the analyte-binding oligonucleotide comprises (a) a sequence that is hybridizable to the analyte and (b) a sequence that is hybridizable to the stem oligonucleotide;
(ii) the linear stem oligonucleotide comprises (a) a sequence that is hybridizable to the analyte-binding oligonucleotide and (b) a sequence that is directly or indirectly hybridizable to the labeled oligonucleotide;
(iii) the labeled oligonucleotide comprises (a) a sequence that is directly or indirectly hybridizable to the stem oligonucleotide and (b) a label capable of directly or indirectly generating a detectable signal;
(iv) the capture polymer comprises a first portion that is hybridizable to the analyte, said first portion comprising at least one modified nucleotide that enhances strength of hybridization of the polymer to the analyte, and a second portion comprising a material that is not substantially hybridizable to nucleic acid;
(B) detecting or quantitating the complex of step (A);
whereby detection or quantitation of the complex of step (A) is indicative of presence or quantity of the nucleic acid analyte in the sample.

14. A method for detecting or quantitating a nucleic acid analyte in a sample, said method comprising:
(A) contacting said sample with an analyte-binding oligonucleotide, a linear labeled oligonucleotide and a capture polymer under conditions whereby a complex is formed comprising the analyte, analyte-binding oligonucleotide, linear labeled oligonucleotide and capture polymer, wherein:
(i) the analyte-binding oligonucleotide comprises (a) a sequence that is hybridizable to the analyte and (b) a sequence that is hybridizable to the linear labeled oligonucleotide;
(ii) the linear labeled oligonucleotide comprises (a) two or more units of label each attached directly to the oligonucleotide and (b) a sequence that is hybridizable to the analyte-binding oligonucleotide;
(iii) the capture polymer comprises a first portion that is hybridizable to the analyte, said first portion comprising at least one modified nucleotide that enhances strength of hybridization of the polymer to the analyte, and a second portion comprising a material that is not substantially hybridizable to nucleic acid;
(B) detecting or quantitating the complex of step (A);
whereby detection or quantitation of the complex of step (A) is indicative of presence or quantity of the nucleic acid analyte in the sample.

15. A method for detecting or quantitating a nucleic acid analyte in a sample, said method comprising:
(A) contacting said sample with an analyte-binding linear labeled oligonucleotide and a capture polymer under conditions whereby a complex is formed comprising the analyte, analyte-binding linear labeled oligonucleotide and capture polymer, wherein:
(i) the analyte-binding linear labeled oligonucleotide comprises (a) a sequence that is hybridizable to the analyte and (b) two or more units of label each attached directly to the oligonucleotide;
(ii) the capture polymer comprises a first portion that is hybridizable to the analyte, said first portion comprising at least one modified nucleotide that enhances strength of hybridization of the polymer to the analyte, and a second portion comprising a material that is not substantially hybridizable to nucleic acid;
(B) detecting or quantitating the complex of step (A);
whereby detection or quantitation of the complex of step (A) is indicative of presence or quantity of the nucleic acid analyte in the sample.

16. The method of any of claims 1-15, further comprising contacting the sample with a blocker oligonucleotide.

17. The method of any of claims 1-15, wherein the capture polymer is hybridized to an oligonucleotide that is directly attached to a solid or semi-solid support.

18. The method of any of claims 1-15, wherein the capture polymer is directly attached to a solid or semi-solid support.

19. The method of any of claims 1-15, wherein the nucleic acid analyte is selected from the group consisting of RNA, DNA, RNA/DNA hybrid and nucleic acid-protein complex.

20. The method of any of claims 1-15, wherein the nucleic acid analyte comprises a sequence encoding part or all of a polypeptide selected from the group consisting of growth hormone, insulin-like growth factors, human growth hormone, N-methionyl human growth hormone, bovine growth hormone, parathyroid hormone, thyroxine, insulin, proinsulin, relaxin, prorelaxin, glycoprotein hormones, follicle stimulating hormone (FSH), thyroid stimulating hormone (TSH), leutinizing hormone (LH), hematopoietic growth factor, vesicular endothelial growth factor (VEGF), hepatic growth factor, fibroblast growth factor, prolactin, placental lactogen, tumor necrosis factor-alpha, tumor necrosis factor-beta, mullerian-inhibiting substance, mouse gonadotropin-associated peptide, inhibin, activin, vascular endothelial growth factor, integrin, nerve growth factors (NGFs), NGF-beta, platelet-growth factor, transforming growth factors (TGFs), TGF-alpha, TGF-beta, insulin-like growth factor-I, insulin-like growth factor-II, erythropoietin (EPO), osteoinductive factors, interferons, interferon-alpha, interferon-beta, interferon-gamma, colony stimulating factors (CSFs), macrophage-CSF (M-CSF), granulocyte-macrophage-CSF (GM-CSF), granulocyte-CSF (G-CSF), thrombopoietin (TPO), interleukins (ILs), IL-1, IL-1alpha, IL-2, IL-3, IL-4, IL-5, IL-6, IL-7, IL-8, IL-9, IL-11, IL-12, LIF, SCF, neurturin (NTN), kit-ligand (KL), HER2, human Fc, human heavy and light chains (constant region), KDR, nitric oxide synthase (NOS) and angiotensin converting enzyme (ACE).

21. The method of any of claims 1-15, wherein the sample is selected from the group consisting of blood, serum, sputum, urine, semen, cerebrospinal fluid, bronchial aspirate, organ tissue, cell lysate and cell culture medium.

22. The method of any of claims 1-15, wherein the sequence of the analyte-binding oligonucleotide that is hybridizable to the analyte is a sequence that is complementary to a sequence of the analyte.

23. The method of any of claims 2, 3, 5, 6, 8, 9, 11, 12, 14 and 15, wherein two tandem units of label of the linear labeled oligonucleotide are separated by at least about 1, 3 or 5 nucleotides.

24. The method of any of claims 2, 3, 5, 6, 8, 9, 11, 12, 14 and 15, wherein two tandem units of label of the linear labeled oligonucleotide are separated by from about 1 to about 12 nucleotides.

25. The method of any of claims 2, 3, 5, 6, 8, 9, 11, 12, 14 and 15, wherein two tandem units of label of the linear labeled oligonucleotide are separated by from about 3 to about 10 nucleotides.

26. The method of any of claims 2, 3, 5, 6, 8, 9, 11, 12, 14 and 15, wherein two tandem units of label of the linear labeled oligonucleotide are separated by from about 5 to about 8 nucleotides.

27. The method of any of claims 2, 3, 5, 6, 8, 9, 11, 12, 14 and 15, wherein the label is attached by covalent bond to the linear labeled oligonucleotide.

28. The method of any of claims 1-15, wherein the label of the labeled oligonucleotide is selected from the group consisting of an antigen, a member of a specific binding pair, a fluorescent dye and a member of a reporter-quencher pair.

29. The method of claim 28, wherein said antigen is selected from the group consisting of digoxigenin, biotin and fluorescein isothiocyanate.

30. The method of claim 28, wherein said specific binding pair is selected from the group consisting of a receptor-ligand pair and an enzyme-substrate pair.

31. The method of claim 28, wherein said fluorescent dye is fluorescein isothiocyanate, rhodamine or Texas Red.

32. The method of claim 28, wherein the reporter-quencher pair comprises dyes capable of fluorescent resonance energy transfer.

33. The method of any of claims 1-15, wherein the labeled oligonucleotide is detected by contacting the labeled oligonucleotide with a compound that binds to the labels of the labeled oligonucleotide, wherein said compound is capable of directly or indirectly generating a detectable signal.

34. The method of any of claims 1-15, wherein capture polymers are provided as an array.

35. The method of any of claims 4, 6-9 and 13-15, wherein the material that is not substantially hybridizable to nucleic acid is inert carbon.

36. The method of claim 35, wherein the inert carbon is provided as ethylene glycol.

37. The method of claim 36, wherein said ethylene glycol has the chemical structure 18-O-Dimethoxytritylhexaethyleneglycol, 1-[(2-cyanoethyl)-(N,N-diisopropyl)]-phosphoramidite.

38. The method of any of claims 4, 6-9 and 13-15, wherein the capture polymer comprises a spacer component.

39. The method of claim 38, wherein the spacer component comprises at least one C18 spacer.

40. The method of claim 39, wherein the spacer component comprises at least three C18 spacers.

41. The method of claim 40, wherein the spacer component comprises at least four C18 spacers.

42. The method of claim 39, wherein the spacer component comprises from about 1 to about 8 C18 spacers.

43. The method of claim 42, wherein the spacer component comprises from about 3 to about 6 C18 spacers.

44. The method of claim 38, wherein the spacer component is the material that is not substantially hybridizable to nucleic acid of the second portion of the capture polymer.

45. The method of any of claims 5, 6 and 10-15, wherein the capture polymer comprises at least 3 said modified nucleotide.

46. The method of claim 45, wherein the capture polymer comprises at least 5 said modified nucleotide.

47. The method of claim any of claims 5, 6 and 10-15, wherein at least 10 percent of the total number of nucleotides in the capture polymer are said modified nucleotide.

48. The method of claim 47, wherein at least 20 percent of the total number of nucleotides in the capture polymer are said modified nucleotide.

49. The method of claim 48, wherein at least 30 percent of the total number of nucleotides in the capture polymer are said modified nucleotide.

50. The method of claim 49, wherein at least 40 percent of the total number of nucleotides in the capture polymer are said modified nucleotide.

51. The method of claim 50, wherein at least 50 percent of the total number of nucleotides in the capture polymer are said modified nucleotide.

52. The method of claim 47, wherein from about 10 to about 50 percent of the total number of nucleotides in the capture polymer are said modified nucleotide.

53. The method of any of claims 5, 6 and 10-15, wherein the modified nucleotide is 2′-O-methoxy-RNA or derivative thereof.

54. The method of any of claims 5, 6 and 10-15, wherein at least one modified nucleotide is located in each of the 5′ and 3′ regions of the sequence that is hybridizable to the analyte.
